(19) United States
(12) Patent Application Publication
(10) Pub. No.: US 2007/0059685 A1
(43) Pub. Date: Mar. 15, 2007

(54) Method for producing improved results for applications which directly or indirectly utilize gene expression assay results

(76) Inventor: David E. Kohne, San Diego, CA (US)

Correspondence Address: WESLEY B. AMES, 7031 LOS VIENTOS SERENOS, ESCONDIDO, CA, 92029, US

(21) Appl. No.: 11/453,298

(22) Filed: Jun. 13, 2006

Related U.S. Application Data
(63) Continuation-in-part of application No. 11/421,961, filed on Jun. 2, 2006.
(60) Provisional application No. 60/689,985, filed on Jun. 13, 2005.
(60) Provisional application No. 60/687,526, filed on Jun. 3, 2005.

(51) Int. Cl. C12Q 1/70; C12Q 1/68; G06F 19/00

(52) U.S. Cl. 435/5; 435/6; 702/20

(57) ABSTRACT

A method for producing improved results for applications of any kind which directly or indirectly utilizes gene expression assay results.

### RELATED APPLICATIONS

This application claims the benefit of Kohne, U.S. Provisional Application No. 60/689,985, filed Jun. 13, 2005, and is a continuation-in-part of Kohne, U.S. application Ser. No. 11/421,961, filed Jun. 2, 2006, which claims the benefit of Kohne, U.S. Provisional Application No. 60/687,526, filed Jun. 3, 2005, each of which is incorporated herein by reference in its entirety.

### FIELD OF THE INVENTION

The present invention relates to methods for obtaining gene expression results which are improved through the use of enhanced normalization, and the use of those improved results in additional gene expression-based methods and analyses.

### BACKGROUND OF THE INVENTION

The following discussion is provided solely to assist the understanding of the reader, and does not constitute an admission that any of the information discussed or references cited constitute prior art to the present invention.

Prior art gene expression analysis methods and the production of prior art assay measured particular gene RNA transcript number (RN) (i.e., the RN for a particular gene RNA transcript which is associated with the amount of cell sample or standard RNA which is in the assay RT step, is equal to the number of particular gene RNA transcript molecules which is present in the assay RT step) and abundance and normalized assay signal for a particular gene RNA transcript expression assay result (NAS) and other equivalent or similar results, as well as prior art gene expression analysis comparison methods and the production of prior art gene comparison assay measured particular gene NAS ratio (NASR) and normalized differential gene expression ratio (N-DGER) and other equivalent results were extensively described and discussed in a prior filed provisional patent application Ser. No. (148) and co-pending U.S. patent application Ser. No. 11/421,961, and in references cited in those document and cited herein. That submitted provisional patent application and non-provisional applications and their cited references are incorporated by reference in their entireties into the present patent application.

A goal of gene expression analysis is to utilize the measured gene expression assay results to find one or more relationships or patterns in the analysis results, which have utility for one or more purposes. For the prior art such a relationship can be simple, and involve the direct comparison of the gene expression analysis assay measured RN or abundance values or normalized assay signal (NAS) values or their equivalents, for particular genes associated with one or more biological samples, in order to produce a particular gene comparison N-DGER or NASR value. As discussed in the provisional patent application Ser. No. (148) and U.S. patent application Ser. No. 11/421,961, a prior art assay measured particular gene NASR value is believed to equal the N-DGER value for the particular gene comparison. For such a particular gene comparison the N-DGER value may represent the comparison of the extent of expression of: the same particular gene in different cell samples, i.e. an SGDS comparison; different particular genes in different cell samples, i.e. a DGDS comparison; or different genes in the same cell sample, i.e. a DGSS comparison.

Prior art believes and practices that such particular gene N-DGER values are biologically correct, and that the N-DGER value obtained can be used to indicate the quantitative difference in gene expression extent between the compared particular genes, and to infer the correct direction of gene regulation which exists for the comparison. That is, whether a compared gene is up- or down-regulated. The regulation direction and/or the quantitative gene expression extent difference for the comparison, are routinely used by the prior art to characterize the effect of different compounds, treatments, and situations, on the expression of different particular genes and groups of particular genes in for cell sample and for a cell sample comparison. Such information is often used by the prior art to identify particular genes which may be drug candidates, particular genes which are associated with a particular disease situation, particular genes which are affected by particular toxic or non-toxic compounds of any kind, particular genes which are associated with a particular therapeutic process or compound, particular genes which are associated with a particular biological state such as a cell cycle or differentiated state or some other state, and many other purposes. Such prior art usage of particular gene N-DGER values can involve just one particular gene N-DGER value, or two or more such values. The use of multiple particular gene N-DGER values can require complex analysis. Prior art has analyzed a large number of biological situations by comparing the particular gene N-DGER values. These include but are not limited to, each invention related area described herein.

In addition a prior art assay measured particular gene abundance or RN or RAS result for a cell sample is also believed to be biologically accurate by the prior art. Prior art often produces and further analyses these results for multiple particular genes in one cell sample to produce a gene expression profile, herein termed a GEP, for the cell sample. Such prior art produced GEPs are also believed to be biologically accurate by the prior art. Prior art often further analyses gene expression profiles for diseased, treated, different and normal cell samples in order to identify one or more particular genes whose expression is characteristic for or specific for the diseased treated, different, or biological state (57-63, 82-97). Such gene expression profiles are also further compared and/or analyzed in order to discover and identify gene expression regulation pathways.

Prior art measured gene expression abundance and/or RN and/or NAS and/or NASR and/or N-DGER results are often used directly or indirectly in a further application, herein termed a higher order application, to produce higher order application results. As an example, prior art measured gene expression assay abundance and/or RNA and/or NAS and/or NASR and/or N-DGER results are often directly and indirectly used by the prior art in further applications which analyses these results to produce further application results. This is an example of the direct use of prior art measured gene expression assay results in a further application. Herein an application which directly utilizes measured gene expression assay measured abundance and/or RN and/or NAS and/or NASR and/or N-DGER results is termed a zero order application result, and a zero order application is a higher order application. Prior art also often directly or indirectly uses zero order application results in a further application, herein termed a first order application, to produce first order application results. A first order application is also a higher order application. Thus, a first order application directly uses zero order application results, and indirectly uses gene expression assay measured abundance and/or RNA and/or NAS and/or NASR and/or N-DGER results. Examples of the higher order application use of prior art gene expression results are discussed below.

Prior art measured gene expression abundance and/or RN and/or NAS and/or NASR and/or N-DGER results from one or another of these invention related areas, are often further analyzed by more complex analysis methods. Such a prior art gene expression analysis result can be supervised or unsupervised. For supervised analysis each gene expression result is associated with one or more biological or other characteristic, and then analyzed. A typical example of a supervised analysis is the classification of biological samples. Such analysis has been used widely in disease situations, especially for the cancer area. Unsupervised analysis involves looking for structure and relationships in the gene expression data itself. A typical example of such unsupervised analysis is gene clustering, i.e. discovering sets of genes with similar expression patterns. Another example is sample clustering, i.e. discovering which biological samples are similar in terms of similarly expressed genes. A further example is principle component analysis, i.e. discovering the axes of greatest variability. Supervised and unsupervised analyses can be done together on a gene expression data set, and this approach is termed partially supervised analysis. At present cluster analysis is the most widely used method of gene expression data analysis, and there are a variety of effective cluster analysis methods. References 9 and 10, as well as others, present extensive discussions on supervised and unsupervised gene expression data analysis methods which are utilized by the prior art.

References 1-12 contain a variety of discussions, examples, and references pertaining to unsupervised or supervised gene expression data analysis. References 1-147 present a variety of different biological, medical, pharmaceutical, basic research, manufacturing, agricultural, and industrial, associated analyses of these types. The purpose of each of these analyses as well as others, involves one or more of the following. Classifying biological samples. Defining cell or organism regulatory pathways and networks. Defining disease pathways and networks. Defining disease susceptibilities. Defining the response to therapy. Predicting response to therapy, and other clinical outcomes. Determining compound and treatment toxicity. Determining response to genetic changes. Drug discovery, development, manufacturing and use. Determining metabolic interrelationships and regulatory pathways and networks. Determining a wide variety of systems biology interrelationships and insights. Such analyses represent only a fraction of the uses which are possible. Examples of many of these and other uses are described in one or another of references 1-147.

### SUMMARY OF THE INVENTION

Recent developments in a range of biological studies and in medicine have heavily utilized gene expression studies directly or indirectly in order to distinguish processes and effects at the cellular level, and to apply those results in further applications. An example of such further applications are diagnostic and therapeutic treatment evaluations and course of treatment decisions.

The present invention contributes heavily to the effectiveness and reliability of such applications of gene expression determinations through the utilization of gene expression study and application results which are improved through the use of improved normalization practices.

Thus, the in first aspect, the invention concerns a method for producing an improved gene expression profile(GEP) for one or more cell samples. The method involves determining one or more particular gene(PG) improved results(IR) for at least one cell sample; and compiling the PG IR values to produce one or more forms of improved GEP for said cell sample.

In certain embodiments of this aspect as well the other aspects described for the invention, the cells in the cell sample can be of essentially any type, for example, normal cells; abnormal cells; untreated cells; treated cells; physically treated cells; chemically treated cells; drug treated cells; bioactive compound treated cells; cells from a psychologically treated individual; drug candidate treated cells; toxic compound treated cells; differentiated cells; undifferentiated cells; biological agent infected cells; virus infected cells; cells from an individual infected by a pathogenic bacterium; cells from an individual infected by a eucaryotic microbe; neoplastic cells; cancer cells; diseased cells; pathological cells; in vitro cultured cells; in vitro cultured cells of an immortalized cell line; in vivo sampled cells; in vivo sampled cells of a particular tissue; prokaryotic cells; eucaryotic cells; temporally treated cells; mammalian cells; mouse cells; rat cells; and human cells. Cells can further be of a particular age, development stage, nutritional status.

In certain embodiments of this aspect and the other aspects below in which gene expression results and/or improved GEPs are obtained and/or used, one or a plurality of cell samples can be analyzed, e.g., at least 2, 3, 5, 10, 20, 50, 100, 200, 300, 500, 1000, or even more or a number in a range of 1-5, 2-10, 11-50, 51-100, 101-500, 501-1000, 1000-10000, among others.

In particular embodiments of the above aspect and the aspects below in which improved results and/or improved GEPs are obtained and/or used, improved results(IR) for a plurality of different PGs are obtained and/or used, e.g., at least 2, 3, 5, 10, 20, 50, 100, 200, 300, 500, 1000, or even more or a number in a range of 1-5, 2-10, 11-50, 51-100, 101-500, 501-1000, 1000-10000, among others.

In certain embodiments of the above aspect and other aspects below in which improved results and/or GEPs are obtained and/or used, PGs of the cell sample GEP include at least one cellular RNA, e.g., mRNA, siRNA, miRNA, regulatory RNA; the GEP is improved in quantitative accuracy, qualitative accuracy, or both, interpretability, reproducibility, intercomparability, and/or utility as compared to a GEP compiled from results which are not IRs; determination of one or more particular geen improved results is performed using one or more microarray assays, RT-PCR, an affinity binding media, nuclease protection method, and/or a clone counting method.

Another aspect concerns a method for identifying a particular cell sample type of interest, and involves comparing gene expression profiles (GEPs) of at least one cell sample type of interest and at least one reference cell sample type of interest, and identifying the cell sample type of interest based on best match comparison of the respective GEPs, e.g., GEPs which incorporate expression results for a plurality of PGs.

In embodiments of the above aspect and other aspects below which involve the use of GEPs, the method can also include identifying, for a plurality of PGs, which PGs are differentially expressed in the cell sample of interest, and/or utilizing the method of the first aspect above (e.g., as embodiment as indicated or otherwise described herein) to determine a GEP(s) for one or more cell samples of the cell sample type of interest or for one or more cell samples of a specified reference cell sample type of interest, or both, and incorporating those results in at least one GEP.

In certain embodiments of this aspect and the aspects below which involve creation and/or use of GEPs, one or more regulated genes are detectably expressed in both the cell sample of interest and the reference cell sample type; one or more up regulated genes is not detectable as being expressed in one of the cell samples; a cell type is as indicated for the aspect above; the cell sample(s) of interest include a plurality of separate different cell or cell sample types (e.g., at least 2, 3, 4, 5, 7, 10, 20, 30, 50, 2-5, 5-10, 10-20, 20-50, or even more);

Another aspect provides a method for identifying a set of genes which may be used to identify or characterize a particular cell sample type of interest, and includes determining improved gene expression results for a plurality of particular genes (PGs) in the particular cell sample or sample (or cell sample type(s)) of interest and in at least one reference cell sample type; and analyzing and identifying PGs which are differentially expressed in the cell sample of interest compared to the reference cell sample type. The method can also include selecting at least a subset of the differentially expressed genes as the set of genes which may be used to identify or characterize the cell sample or cell sample type of interest.

In certain embodiments, the improved gene expression results are compiled as a GEP (directly or following analysis) for one or more cell samples of the cell samples or cell sample types of interest or for one or more cell samples of a specified reference cell sample type of interest, or both; and the process of identifying PGs which are differentially expressed involves comparing gene expression profiles (GEPs) of at least one cell sample or cell sample type of interest and at least one reference cell sample type of interest.

In particular embodiments, one or more regulated genes are detectably expressed in both the cell sample or cell sample type of interest and the reference cell sample type; one or more up regulated genes is not detectable as being expressed in one of the cell samples or cell sample types; cells of a type as indicated above are used in a cell sample;

Also in particular embodiments, the selecting involves identifying from a set of differentially expressed PGs a discrimination set of one or more PGs which can be used to reliably, selectively, and specifically identify individual cell samples of the type of interest and to distinguish those cell sample types of interest from the specific reference cell sample type. Advantageously the discrimination set can also distinguish the cell sample type of interest from a plurality of other cell sample types (e.g., cell sample types from other organisms, other growth stages, other tissues, cells subjected to other chemical and/or physical treatments, and the like). In certain embodiments, the bases for selection includes the magnitude of the differential expression for a particular gene; the consistency of occurrence and direction of the differential expression for a particular gene or genes.

In particular embodiments of this aspect and other aspects herein involving PG selection for gene discrimination and/or direct or indirect application of GEDs, the selection process or method or the application involves use of a linear discriminant method, a K-nearest neighbor method, a neural network method, a decision tree method, a partially supervised method, a class discovery method, a hierarchical agglomerative clustering method, a hierarchical divisive clustering method, a non-hierarchical K-means method, a self organizing maps and trees method, a principal component analysis method, a relationship between clustering and a principal component method, a gene shaving method, a clustering in discretised space method, a graph based clustering method, a Bayesian model method, a fuzzy clustering method, a clustering of genes and samples method, a data mining analysis method, a systems biology analysis method, an independent component analysis method; and/or a direct comparison method.

A related aspect concerns an improved set of cell sample type discrimination gene set identifier nucleic acid molecules, which includes a set of nucleic acid molecules which provide specific detection of individual particular genes identified by the methods of the preceding aspect, which reliably, selectively, and specifically identify individual cell samples of the type or types of interest, or distinguish the cell sample type or types of interest from at least one specific reference cell sample type, or both, based on improved gene expression results.

In particular embodiments, the set of identifier nucleic acid molecules includes a set of labeled or unlabeled or both hybridization probes, a set of capture oligonucleotides (e.g., incorporated in an oligonucleotide microarray), a set of amplification primers; the molecules provide identification of cells of a cell type as indicated above, e.g., a cancer cells, cells infected by an infectious agent, cells of a developmental state, cells exposed to or treated by a bioactive molecule, and/or cells exposed to a defined environmental condition.

An aspect similar to the aspect two above provides a method for identifying improved sets of particular genes for an application utilizing gene expression results, where the method involves obtaining improved gene expression results for at least one application pertinent gene, and selecting a discrimination gene set based on differential gene expression of the gene in at least one application pertinent cell sample type.

In particular embodiments, such application pertinent cell type (or types) is identified based on a cellular process associated with the application; the discrimination gene set is selected utilizing the method of an embodiment of the aspect two above; the improved gene expression results are obtained utilizing the method of any of the embodiments of the first aspect above.

In embodiments of this aspect and other aspects involving a further application of gene expression results and/or GEPs, the application includes one or more of a data mining analysis, a systems biology analysis, a regulatory pathway identification, or analysis, or monitoring, or any two, or all three, a drug or bioactive compound or biomarker discovery and identification, a drug or bioactive compound or biomarker validation, a drug or bioactive compound or biomarker development, a drug or bioactive compound efficacy analysis, a drug or bioactive compound safety evaluation, a drug or bioactive compound toxicity evaluation, a drug or bioactive compound QA/QC evaluation, a drug or bioactive compound manufacturing monitoring, a drug or bioactive compound or biomarker related diagnostic test development or use or both, a particular cell sample of interest related diagnostic test development or use or both, a disease or pathologic state or both detection or evaluation or both, a disease or pathologic state or both detection or evaluation or both, before and after administration of a therapeutic treatment, a disease or pathologic state or both detection or evaluation or both before and after drug administration, a disease or pathologic state or both detection, monitoring, or prognosis evaluation or any two or all three, a disease or pathologic state or both detection, monitoring, or prognosis evaluation or any two or all three, before or after drug or other treatment or both, a drug or bioactive compound commercial product candidate selection, a drug or bioactive molecule related clinical trial monitoring, a drug or bioactive compound commercial product candidate market segment identification, a drug or bioactive compound effectiveness and safety in the treated patient evaluation, a drug or bioactive compound prescription to the patient selection, and a monitoring of drug or biomolecule effectiveness or toxicity or both in the treated patient, where the monitoring may be long or short term or both.

Also in certain embodiments, the method also includes providing a set of a set of particular gene identifier nucleic acid molecules (e.g., as described for the preceding aspect), where members of the set of nucleic acid molecules provide specific detection of corresponding members of said discrimination gene set; such set can, for example, include at least 2, 3, 4, 5, 7, 10, 15, 20, 50, or even more different identifier nucleic acid molecules.

The invention also concerns as aspect providing a method for producing improved results for an application which directly or indirectly utilizes at least one gene expression profile for at least one particular gene, where the method involves utilizing at least one improved gene expression profile (GEP) directly or indirectly in the application, thereby producing improved application results.

In particular embodiments, the GEP is produced according to an embodiment of the first aspect above; the GEP is a particular cell sample or cell sample type GEP; the GEP includes a cell sample GEP which includes a set of one or more regulated PGs which may potentially be used (or can be used or are actually used) to selectively and specifically identify a particular cell sample type or a particular cell sample type physiological state (PS) of interest or both.

Particular embodiments involve use of an analysis method as indicated above; the application includes a component or components as indicated above.

Another aspect concerns an improved method for identifying regulated particular genes (PGs) which are regulated in response to exposure to a particular treatment. The method involves comparing at least one improved gene expression profile (GEP) (e.g., produced by the method of an embodiment of the first aspect above) incorporating improved results for at least one cell sample exposed to the treatment with at least one improved gene expression profile for at least one reference cell sample, thereby identifying PGs with differential expression in the treated cell sample, and can also include subjecting cells in the treated cell sample to the treatment and cells of the reference cell sample are not subjected to the treatment, e.g., the sample and reference cells can be matched except for the treatment.

The method can also involve utilizing one or more selection processes to identify and rank the regulated PGs based on the magnitude and direction of the change in expression level for PGs in the treated cell sample.

The method can also include utilizing one or more further selection processes to evaluate the suitability of each of the regulated PGs for the purpose of the comparison, and interpreting and ranking and arranging the members of the set of regulated PGs and their characteristics in a manner which reflects their suitability of use for the purpose of the said comparison and identification; the selection process can include one or more analysis techniques as indicated above.

The method can also include exposing at least one of a plurality of matching cell samples to a treatment of interest thereby forming a treated cell sample, while at least one other of said cell sample portions is not exposed to said treatment of interest, and constitutes a reference sample, and using the method of an embodiment of the first aspect to produce a GEP for each of the cell samples.

In particular embodiments of this aspect and other aspects herein which involve treatment of cells, the treatment can include one or more (e.g., any combination of the listed treatments taken 2, 3, 4 at a time) of exposure to a compound in a compound screening library, exposure to a pharmaceutical drug screening hit, exposure to a pharmaceutical drug lead, exposure to a pharmaceutical drug, exposure to a potentially toxic compound, exposure to a toxic compound, exposure to an illegal drug, exposure to nucleic acid binding compound, exposure to an infectious agent, exposure to a virus, exposure to a bacterium, exposure to radiation, exposure to light, exposure to ultraviolet light, exposure to a temperature shift, exposure to a biological stress condition, exposure to a psychological stress condition, exposure to a physical condition, exposure to a bioactive compound, and exposure to an environmental condition; the regulated genes are as indicated above; the cell type(s), PGs, RNA, improvement type, and/or assay type is as indicated above; the process of selecting genes and/or discrimination sets of PGs is as indicated above; a set of particular gene identifier nucleic acid molecules as indicated above is provided.

In view of he various applications of the improved gene expression results, improved GEPs, and associated improved applications, another aspect concerns a method for producing higher order application results which are improved in one or more of qualitative accuracy, quantitative accuracy, interpretability, reproducibility, intercomparability, and utility, relative to prior art produced higher order application results, by using a method for producing improved GEPs as described for aspects and embodiments above to produce improved results, and utilizing one or more of those improved results directly or indirectly in a higher order application to produce higher order application results which are improved in one or more of qualitative accuracy, quantitative accuracy, interpretability, reproducibility, intercomparability, and utility, relative to prior art produced higher order application results.

In certain embodiments, the higher order application is an application or includes an application component as indicated above

Yet another aspect concerns a method for producing improved information and results concerning the physiological state of cells in a cell sample (e.g., from an individual organism or tissue, or type of organism or tissue) of a particular cell type of interest. The method involves utilizing one or more particular physiological state gene expression profiles (PS GEPs) to identify the physiological state of different samples of the particular cell type of interest, where particular PS GEPs for the particular cell type of interest selectively distinguish a particular physiological state(PS) for the particular cell type of interest, and where the PS GEPs are improved by the incorporation of improved gene expression results and where the information and results are improved in one or more of qualitative accuracy, quantitative accuracy, interpretability, reproducibility, intercomparability, and utility, relative to prior art produced information and results.

The method can also include monitoring the physiological state and analyzing the monitoring results to evaluate and determine the physiological state of the particular cell type sample of interest over time and/or under changing or changed conditions.

In particular embodiments, the method of an embodiment of the first aspect above (and/or other above aspect) is utilized to produce one or more physiological state gene expression profiles (PS GEPs) for the particular cell type of interest which selectively distinguish a particular physiological state(PS) for the particular cell type of interest.

In certain embodiments,t eh cell type is as indicated above; the cell type is a eucaryotic cell type, a prokaryotic cell type, a plant cell type, a bacterial cell type, a pathogenic bacterial cell type, a yeast cell type, a fungal cell type, a mammalian cell type, a human cell type, an in vitro grown cell type, an immortalized cell line type, an in vivo grown cell type, an infectious organism or agent infected cell type, a virus infected cell type, a genetically modified cell type, and/or an in vivo or in vitro cell type used for producing or manufacturing a pharmaceutical agent or protein or small molecule or lipid.

In certain embodiments, the particular physiological state is or includes a cell cycle stage related PS, a cell growth state related PS, a cell size related PS, a differentiated state related PS, an undifferentiated state related PS, a toxic state related PS, a cell age related PS, an infectious state related PS, a nutritional state related PS, a drug or bioactive agent treatment of the cell type related PS, an environmental state related PS, a physical treatment of the cell type related PS, a psychological treatment of the cell type related PS, a chemical treatment of the cell type related PS, and/or a hormone treatment related PS.

The invention also concerns an aspect providing a method for producing improved clinical trial information and results (or therapeutic evaluation information and results) which are improved in qualitative accuracy, quantitative accuracy, interpretability, reproducibility, intercomparability, or utility, relative to prior art produced such information and results, for the evaluation of one or more or all of the safety, dose, or efficacy of a drug or bioactive agent (BA), where the method includes monitoring one or more improved gene expression profiles (GEPs) for drug or BA treated and untreated particular cell types of interest respectively for the appearance of one or more drug or BA treatment desired effects or undesired effects or both in the treated cell types of interest, where the improved GEPs incorporate improved gene expression results.

Certain embodiments also include analyzing the results of the monitoring to evaluate the safety, dose, and efficacy of the drug or BA treatment of the particular cell types of interest, and/or include using the method of an embodiment of the first aspect to produce one or more of the particular GEPs.

In particular embodiments, the cell type(s) is or includes a cell type as indicated above; the cell type is or includes eucaryotic, prokaryotic, plant, bacteria, yeast or fungus, mammalian, human, cell types infected with a biological or other infectious agent, normal cell types, abnormal, untreated, treated, psychological treated, toxic compound treated, differentiated, undifferentiated, neoplastic, in vitro grown, in vivo, diseased, and pathologic.

In particular embodiments the GEP is or includes a complete GEP for the treated and untreated cell type or types of interest; the GEP is or includes a partial GEP specific for a particular treated or untreated cell type or types of interest; the GEP is or includes a combination complete and partial GEPs for the treated or untreated cell type or types of interest; the desired or undesired effect is or includes the known desired effects of the drug or BA on the cell types of interest, the unknown potential desired effects of the drug or BA on the cell types of interest, the known undesired effects of the drug on the cell types of interest, or the unknown potential undesired effects on the cell types of interest.

A further aspect concerns a method for producing improved information and results concerning the efficacy and toxicity or both or the desired and undesired effects or both, of treatment for a patient being treated with a particular drug or bioactive agent (BA), or with a combination of a plurality of drugs or BAs or both, which is improved in one or more of qualitative accuracy, quantitative accuracy, interpretability, reproducibility, intercomparability, and utility, relative to such prior art produced information and results. The method involves monitoring one or more improved gene expression profiles (GEPs) of patient cell samples for drug or BA treated particular cell types of interest for the appearance and/or level of one or more drug treatment desired effects or undesired effects or both in said treated cell types of interest, where the improved GEPs incorporate improved gene expression results. The method can be performed for a plurality of patients, e.g., at least 2, 3, 5, 7, 10, 20, 30, 50, 100, or more.

In particular embodiments the method also includes analyzing the monitoring results to determine the effectiveness of the treatment or undesired effects of the treatment or both, and/or using a method of an embodiment of the first aspect above to produce cell type specific GEPs for the combination of the patient cell types of interest and drug or BA of interest, and/or comparing at least one GEP for a treated cell sample from the patient with at least one untreated cell sample.

In certain embodiments, treated cell sample or the untreated cell sample or both is from the patient; a GEP is or includes a partial GEP specific for a particular treated or untreated cell type or types of interest; the GEP is or includes a combination complete and partial GEPs for the treated or untreated cell type or types of interest; the desired or undesired effect is or includes the known desired effects of the drug or BA on the cell types of interest, the unknown potential desired effects of the drug or BA on the cell types of interest, the known undesired effects of the drug on the cell types of interest, the unknown potential undesired effects on the cell types of interest.

Similarly, as related aspect concerns a method for producing improved patient bioactive agent treatment related health care, and involves utilizing the method of any the embodiments of the preceding aspect to determine the effectiveness of the particular drug or bioactive agent (BA) treatment in a patient, and selecting a drug or BA treatment utilizing the determination of effectiveness information.

In particular embodiments, the selecting is or includes continuation of treatment with said drug or bioactive agent, an increase in dosage of said drug or bioactive agent, a decrease in dosage of said drug or bioactive agent, termination of treatment with said drug or bioactive agent, administration of an additional drug or bioactive agent.

For certain embodiments, the effectiveness information includes information on the efficacy of drug or bioactive agent in the patient, information on the safety of the drug or bioactive agent in the patient, and/or tolerance of dosage level information in said patient; the bioactive agent is a food, nutritional supplement, or nutritional compound.

Yet another related aspect concerns a method for producing improved patient bioactive agent treatment related health care, and involves selecting treatment for a patient based on comparison of at least one improved GEP for the patient, and at least one reference GEP indicative of patient response to the drug or bioactive agent treatment. The GEP for a patient can be produced by the method of an embodiment of the first aspect above.

In particular embodiments, the patient suffers from a disease or condition for which the presence of certain allelic variants is indicative of variation in the effectiveness of treatment with the drug or bioactive agent or indicative of differences in effectiveness of different drugs or bioactive agents; the method of an embodiment of the aspect two above is used to determine the effectiveness of the particular drug or bioactive agent (BA) treatment in a patient, and can further involve utilizing the determination of effectiveness information to select a drug or BA treatment.

As indicated above, in particular embodiments, the selecting is or includes continuation of treatment with the drug or bioactive agent, an increase in dosage of the drug or bioactive agent, a decrease in dosage of the drug or bioactive agent, termination of treatment with said drug or bioactive agent, or administration of an additional drug or bioactive agent.

Also as indicated above, in certain embodiments, the effectiveness information is or includes information on the efficacy of the drug or bioactive agent in the patient, information on the safety of the drug or bioactive agent in the patient, and/or tolerance of dosage level information in the patient.

In yet another aspect, the invention concerns an electronic representation of an improved gene expression profile, including electronic representations of a plurality of improved results and/or GEPs obtained by the method of an embodiment of the first aspect above and/utilizing embodiments of any of the additional embodiments above which can be used in producing an improved GEP. The representation may be visible and/or may be recorded in computer memory and/or in a computer accessible data storage device.

Similarly a related aspect provides a method for determining improved application results for an application which directly or directly utilizes improved gene expression profile (GEP) information, where the method involves entering data describing or derived from said GEP in computer accessible form, operating on that data with a computer program comprising program steps to calculate the application results. The application may be any of the applications described herein and/or may include analysis components as indicated for aspects above.

Additional embodiments will be apparent from the Detailed Description and from the claims.

### DETAILED DESCRIPTION OF THE PREFERRED EMBODIMENTS

The present invention concerns obtaining and using improved results from gene expression assays, including, for example, the use of gene expression profiles. Such improved results are obtained due to applying correct normalization to the assay results.

In order to assist the reader, a number of terms used frequently in this description are provided in the glossary table below. Additional terms are defined in parent application Ser. No. 11/421,961.

### GLOSSARY OF SELECTED TERMS

| Term | Description |
|---|---|
| Abundance | Refers to the number or average number of a particular gene's RNA |
| | molecules per cell for a cell sample. |
| DGE/DGER | Differential gene expression generally refers to the concept that the |
| | same particular gene can be expressed to a different extent in |
| | different cells. In addition different particular genes in different |
| | cells, and different particular genes in the same cell, can also be |
| | differentially expressed. Such a difference in gene expression |
| | between compared particular genes is generally described in terms |
| | of a DGE ratio or DGER. |
| DGDS/ | Different genes different cell sample (DGDS), and different gene |
| DGSS | same cell sample (DGSS). DGDS designates the comparison of the |
| | expression extents of different particular genes from different cell |
| | samples. DGSS designates the comparison of the expression extents |
| | of different particular genes in the same cell sample. (see also |
| | SGDS). |
| GEP | Gene Expression Profile. A GEP for a cell or cell sample is |
| | composed of the expression results for a set of one or more genes |
| | which are present in a cell type. A GEP for a cell may represent all of the |
| | genes in a particular cell or just a subset of genes, e.g., the set |
| | of genes whose expression pattern is specific for a particular |
| | diseased, treated, differentiated, or physiological state of the |
| | particular cell type. |
| IR | Improved Results. Refers to gene expression assay measured |
| | abundance, RN, or NAS results, and gene expression comparison |
| | assay measured NASR and N-DGER results which are improved by |
| | the methods described in reference 148 and U.S. application |
| | 11/421,961. |
| NAS/NASR | Normalized assay signal for a particular gene RNA transcript |
| | expression assay result. The NAS for a particular gene RNA |
| | transcript expression analysis in an assay is derived by normalizing |
| | the assay measured raw assay signal activity (RAS) associated with |
| | a particular gene RNA transcript expression assay for pertinent |
| | assay variable, and/or assay variable associated NFs. For a |
| | particular gene RNA transcript comparison, the NASR value is |
| | equal to the ratio of the compared particular gene NAS values. A |
| | particular gene assay measured and normalized NASR value will |
| | equal the particular gene True DGER(T-DGER) value when the |
| | NASR value is validly and completely normalized for all pertinent |
| | assay variables. Prior art produced particular gene NASR values are |
| | believed to be biologically accurate, and therefore equal to the |
| | particular gene T-DGER value. |
| N-DGER | Refers to the gene expression comparison assay measured and |
| | normalized DGER for a particular gene. For such a prior art assay, |
| | by definition the NASR = N-DGER for a particular gene comparison. |
| RN | The RNA transcript number. The RN for a particular gene RNA |
| | transcript which is associated with the amount of cell sample or |
| | standard RNA which is in the assay RT step, is equal to the number |
| | of particular gene RNA transcript molecules which is present in the |
| | assay RT step. |
| SGDS | Same gene different sample. SGDS designates the comparison of |
| | the expression extents of the same gene in different cell samples. |
| | (see also DGDS and DGSS) |

As used herein, the term “bioactive agent” refers to any treatment which produces a change or response at the cellular level for at least some cell types. Such treatments can include exposure to a chemical compound or compounds, exposure to radiation and/or light, elevated temperature, and the like.

The terms “cell type” and “cell sample type” are used broadly to refer to cells which differ in at least one distinguishable parameter, and, unless indicated to the contrary, such types are not limited to distinctions between cells from different organisms and/or different differentiated cell classes.

A prior art higher order application, including those described above, can produce biological and/or other conclusions which are known to be valid and correct only when it is known that the gene expression results used or incorporated into the analysis are correct. As discussed extensively in the reference 148 and U.S. patent application Ser. No. 11/421,961, prior art believes and practices that the prior art produced gene expression results are correct. Similarly, prior art also generally believes and practices that the prior art produced gene expression result analyses which utilize the prior art gene expression results, are also correct. Such prior art produced gene expression results may comprise gene expression analysis assay measured particular gene RN values or particular gene abundance values, or particular cell sample GEPs, or particular gene NAS values or equivalent values, and/or particular gene comparison NASR values or particular gene comparison N-DGER values, or equivalent values.

As described and discussed extensively in reference 148 and U.S. patent application Ser. No. 11/421,961, essentially all prior art produced gene expression abundance, RN, NAS, NASR, and N-DGER results and the GEPs derived from them can be known to be either incompletely normalized for all pertinent assay variables, or uninterpretable with regard to the extent of normalization for pertinent assay variables. It was further concluded that most of the uninterpretable results are highly likely to be incompletely normalized for pertinent assay variables, and are therefore highly likely to deviate significantly from biological correctness. As a consequence of these conclusions, prior art higher order application results, such as those obtained for simple or supervised or unsupervised or systems biology or other higher order applications described above, have, at best, utilized uninterpretable prior art produced gene expression results for the analysis, and it is highly likely that most of such prior art gene expression results and the GEPS derived from them which are used in these higher order applications are significantly incorrect. Therefore, essentially all said prior art produced higher order application results are, at best, uninterpretable with regard to biological correctness, and are highly likely to be biologically incorrect. For such prior art higher order application results, it cannot be known whether any specific aspect of the results is right or wrong unless some independent source of information can clarify the issue. Put another way, the direct or indirect use of prior art produced gene expression assay measured abundance and/or RN and/or NAS and/or NASR and/or N-DGER results which are incorrect or uninterpretable in a higher order application, produces prior art higher order application results which are incorrect or significantly sub-optimal with regard to interpretability and correctness.

Methods and means for producing gene expression analysis assay results which can be known to be significantly improved in normalization for pertinent assay variables and improved in interpretability, relative to prior art produced gene expression analysis assay results, are discussed and extensively described in reference 148 and U.S. patent application Ser. No. 11/421,961. Such improved gene expression assay results comprise improved gene expression analysis assay measured particular gene RN values or particular gene abundance values or particular gene NAS values or equivalent values, and/or improved particular gene comparison N-DGER values or particular gene comparison NASR values or equivalent values.

For simplicity, the term higher order application will herein refer to any prior art known or any unknown method which directly or indirectly uses gene expression results to produce higher order application results. Such gene expression results which are directly or indirectly used in a higher order application may comprise gene expression assay measured particular gene RN results and/or particular gene abundance results, and/or particular gene NAS results and/or equivalent results, and/or particular gene comparison NASR results or particular gene comparison N-DGER results or equivalent results. Such higher order applications may refer to one or more of the above described simple particular gene comparison analysis methods, or one or more of the above described supervised or unsupervised analysis methods such as the various types of cluster analysis and principle component analysis, or one or more of the systems biology analysis methods, or one or more other known or unknown gene expression result analysis methods.

Prior art gene expression assay results and the results of the analysis of such gene expression assay results, are often used directly or indirectly for other higher order applications and purposes by the prior art to produce higher order applications and results. Examples of such applications are presented herein. Such higher order applications are very broad and include many aspects of drug discovery, development, validation, manufacturing, marketing, prescription, and use.

The production of improved gene expression analysis assay measured particular gene RN values or particular gene abundance values or particular gene NAS values or equivalent values, and/or the production of improved gene expression comparison analysis assay measured particular gene comparison N-DGER values or particular gene NASR values or equivalent values, are described extensively in the submitted provisional patent application which is entitled “Method for Producing Improved Gene Expression Analysis And Gene Expression Analysis Comparison Assay Results” and corresponding non-provisional U.S. application Ser. No. 11/421,961, and in the Glossary of the present patent application. Said provisional and non-provisional patent applications are incorporated by reference in their entireties into the present application (148).

The terms improved particular gene RN assay result, and improved particular gene abundance assay result, and particular gene NAS assay result, and improved particular gene NASR, and improved particular gene comparison N-DGER assay result, are described extensively in said application, and the terms particular gene RN, abundance, NAS and particular gene comparison N-DGER and NASR are defined in the glossary of said submitted provisional application. Herein such an improved RN, abundance, NAS, N-DGER, or NASR gene expression analysis assay result, or another equivalent assay result, will be referred to as an improved result, or IR, unless otherwise noted.

The present invention relates to any present or future application or process which directly or indirectly utilizes or incorporates or relies on one or more IRs for the application process. Such applications and processes include, but are not limited to, any prior art or future application or process which directly or indirectly utilizes or incorporates or relies on one or more gene expression assay measured particular gene RN values or particular gene abundance values or particular gene NAS values or other equivalent values, and/or gene expression comparison assay measured particular gene comparison NASR values or particular gene comparison N-DGER values or other particular gene comparison equivalent values, for the application or process. Such applications or processes include, but are not limited to, gene expression data analysis methods and algorithms of all kinds and systems biology analysis algorithms and methods of all kinds which directly or indirectly utilize gene expression results, as well as any other use of analysis of gene expression results. Such applications and processes can involve any known or unknown biological life form, including all known or unknown viruses, prokaryotes, and eukaryotes. The invention relates broadly to basic, applied, commercial and industrial research and development of virtually all kinds which have a biological aspect.

More specifically the present invention relates to all areas of basic, applied, commercial, and industrial biological research including, but not limited to, the following. Biochemistry, bioinformatics, biotechnology, cell biology, chemical biology, cell therapy, cell and organ transplantation, developmental biology, ecology, endocrinology, epidemiology, evolution, genetics, gene therapy, genomics, gerontology, immunology, infectious diseases, microbiology, molecular biology, nephrology, neurology, ophthalmology, pediatrics, pharmacology, physiology, plant biology, psychiatry, public health, structural biology, surgery, urology, drug discovery, molecular therapeutics, epidemiology, carcinogenesis, inflammation, pain, nutrition, reproduction, virology, toxicology, pathology, dermatology, gastroenterology, musculoskeletal studies, pregnancy, pulmonary studies, breast cancer, cardiovascular studies, cerebrospinal research, allergy and asthma studies, hepatology, atherosclerosis, diabetes studies, hematology, oncology, osteoporosis studies, rheumatology studies, vaccine studies, circadian rhythms studies, proteome studies, respiratory research, thrombosis studies, anti-viral and anti-microbial and anti-parasite studies gene regulation studies, cell culture studies of all kinds, organ culture and transplant studies of all kinds, protein production studies, and in vitro and in vivo cell and organ growth and differentiation studies of all kinds.

Said invention relates to essentially all areas of marine and terrestrial basic, applied, commercial and industrial agricultural research and development. This includes most of the above mentioned areas as well as the following. Developing improved viruses, microbes, cells, plants and animals, by natural and genetic engineering means, for food production and other purposes. Terrestrial and marine virus, microbe, plant, and animal diseases of all kinds, and disease mechanisms and host-pathogen interactions. Discovery, development, validation, production and use of antiviral agents, anti microbial agents, antifungal agents, pesticides, vaccines of all kinds, plant and animal growth agents, and agricultural pharmaceutical agents of all kinds. Agricultural ecology and toxicology. Products and services which are associated with the above described areas.

Said invention relates to a large number of medical areas, both human and veterinary and these include, but are not limited to, essentially all areas of basic, commercial, industrial, and applied human and veterinary medical research and development. These include, but are not limited to, the following. The pathogenisis and/or prevention and/or diagnosis and/or treatment, and/or cure of: infections and non-infectious diseases of almost all kinds; genetic and non-genetic disease of almost all kinds; nutritional diseases of almost all kinds; central nervous system diseases of almost all kinds, including psychiatric conditions; cancers and tumors of almost all kinds; cardiac diseases of almost all kinds; other tissue, organ, or cell diseases of almost all kinds; inflammation related diseases of almost all kinds; immunologic diseases of almost all kinds; toxic compound related diseases of almost all kinds; addictive diseases of almost all kinds; fetal and developmental diseases of almost all kinds; diagnostic tests for all such diseases; products and services which are associated with the research and development and commercialization related to the diagnosis, prevention, control, treatment, or cure of such diseases, including vaccine development and commercialization.

Said present invention is related to essentially all areas of human and veterinary medicine including those earlier described basic, commercial, or applied biological research areas. Further said present invention is related to essentially all areas of human and veterinary pharmaceutical and basic and applied and commercial and industrial and service research and development, drug discovery and validation and manufacturing, and the re-evaluation of existing drugs or drug rescue, including but not limited to the following areas. Allergy and asthma, addiction, anesthesiology, anti-viral and anti-microbial and anti-fungal and anti-parasite agents, atherosclerosis, biochemistry, blood disorders, cancer and carcinogenesis, cardiology and cardiovascular, cerebrospinal, cell culture, cell therapy, dermatology, diabetes, development, dental, diagnostic, ecology, emergency medicine, endocrinology, epidemiology, gastroenterology, genetics, gene therapy, gerontology, hematology, hepatology, hypertension, immunology and autoimmune disorders, microbiology, molecular medicine, musculoskeletal disorders, nephrology, neurology and neuroscience, nuclear medicine, nutrition, oncology, ophthalmology, otolaryngology, pain, parisitology, pediatrics, pharmaceuticals, psychiatry, psychoses, public health, pulmonary medicine, reproduction, rheumatology, sports medicine, surgery, urology, vaccinology, and virology. Note that the above list of invention related human and veterinary medicine related areas, is by no means a complete list and is included to illustrate that the invention has very very broad application in the human and veterinary and other areas.

Said present invention relates to essentially all of the steps in the process of human and veterinary drug discovery, characterization, optimization, validation, prescription and use. Here the term drug includes antiviral, and microbial, and antifungal compounds, as well as vaccines and other drug and Bioactive molecule types of all kinds. Such invention related steps include, but are not limited to, the following. (a) The identification and characterization and development of one or more biological and/or non-biological drug target discovery systems. (b) Establishing quality control (QC) and quality assurance (QA) methods for each drug target discovery system. (c) The identification and characterization and development of one or more drug target candidates. (d) The identification and characterization and development of one or more biological and/or non-biological systems for screening drug candidates for the target. (e) Establishing QC and QA methods for the drug screening systems. (f) The identification and characterization of one or more drug candidates for each drug target and the evaluation of the specificity, and potency or efficacy and toxicity, of the candidate drug in the drug screening system. (g) The optimization of the drug candidate specificity, potency, and toxicity of the drug candidate in the screening system. (h) The identification and development of diagnostic tests for evaluating the drug candidate and target characteristics. (i) Establishing QC and QA methods for the scale up of synthesis of the drug candidate. (j) The identification, characterization, and development of one or more biological and non-biological systems for further evaluating and optimizing the drug candidate specificity,.potency, efficacy, and toxicity. (k) Establishing QC and QA methods for the biological and non-biological systems. (l) The identification, characterization, and development, of one or more biological systems to evaluate pharmacodynamic and pharmacokinetic characteristics of the candidate drug. (m) Establishing QC and QA methods for this biological system. (n) The use of the biological system to optimize the pharmacodynamic and pharmacokinetic characteristics of the candidate drug. (o) The development of human organism related diagnostic assays for evaluation of the drug candidate pharmacodynamic and pharmacokinetic characteristics, including the specificity, potency, and toxicity, in human organisms. (p) The use of the human related diagnostics to screen human populations for inclusion in clinical trials, and to define the target human population that the drug is effective and safe for. (q) The use of the human organism related diagnostic methods to monitor the pharmacodynamic and pharmacokinetic characteristics of the drug candidate in human trial participants, and for disease monitoring during the clinical trial and after. (t) The use of human related and other diagnostic methods for QC and QA of the drug manufacturing scale up and manufacturing process. (u) the use of human related diagnostic methods to prescribe the drug and monitor the drug effectiveness during treatment and for disease monitoring before, during, and after drug treatment. (v) The use of the human related diagnostic methods to monitor the long term pharmacodynamic and toxicity characteristics.

The invention further relates broadly to basic, commercial, applied, and industrial, research and development and manufacturing and applied and service use of the following. Prokaryote cells and cell cultures, and eucaryotic cells and cultured primary and continuous cells. Eucaryotic organisms, organs, and tissues, as well as organs and tissues cultured in vitro. Such invention related uses include, but are not limited to, the following. The use of prokaryotic cells grown in large quantities to produce a wide variety of products, including drugs. The use of primary and continuous eucaryotic cell cultures grown in large quantities, to produce a wide variety of products, including drugs. The use of prokaryotic and eucaryotic cultured cells for a wide variety of basic, commercial, applied and industrial, research and development and service applications. Such eucaryotic cells include primary and continuous stem cell lines and the use of genetically modified microbial, plant, fungal or animal organisms or cells for various aspects of drug, biochemical, bioproduct or food production. The use of organisms and genetically modified organisms includes interfering RNA treatment of such organisms.

The invention also relates broadly to basic, commercial, applied, and industrial research and development aspects of toxicology. Many if not most of the above described invention related areas also relate directly or indirectly to toxicology, as well as to the areas of quality control and monitoring of water, food quality, nutrition, public health, marine and terrestrial ecology, forensics, and many kinds of technology development, QC and QA, and various services associated with one or more of the above.

Note that the above description of the invention related areas is not a complete list, and represents only a small fraction of the actual invention related areas.

Overall the invention relates to the direct or indirect use of one or more gene expression assay measured invention improved abundance and/or RN and/or NAS and/or NASR and/or N-DGER, herein termed Invention Improved Results or IRs, in a further application which directly or indirectly utilizes IRs, herein termed a higher order application, to produce improved higher order application results, herein termed Improved Application Results or IARs. Said IRs are discussed and defined in reference 148 and U.S. application Ser. No. 11/421,961.

The Underlying Basis of the Invention. As discussed extensively in reference 148 and U.S. application Ser. No. 11/421,961, many prior art produced gene expression assay results can be known to be incorrect, and essentially all of the rest of the prior art produced results are uninterruptible, and most of these are highly likely to be significantly incorrect. This occurs because prior art does not measure or take into consideration for normalization of prior art gene expression assay measured results, multiple pertinent prior art unconsidered assay variables. Further, the prior art normalization process for certain prior art known assay variables which are considered during the prior art normalization process is known to be invalid for many, if not most, prior art gene expression assays. Such prior art produced gene expression assay results are routinely used by the prior art for higher order applications analysis by the earlier discussed gene expression result analysis methods. Because the prior art produced gene expression assay results are significantly incorrect or uninterpretable, the prior art produced higher order application results are also significantly incorrect or uninterpretable, or significantly sub-optimal in correctness and interpretability.

Described extensively in reference 148 and U.S. application Ser. No. 11/421,961 are gene expression assay results which are improved in normalization for pertinent prior art considered and/or prior art unconsidered assay variables. Such improved gene expression assay results are herein termed improved results or IRs.

The underlying basis for the present invention then, is that the use of IRs in a higher order application of any kind, produces higher order application results which can be known to be significantly improved, relative to prior art produced higher order application results. In other words the production of invention improved abundance and/or RN and/or NAS and/or NASR and/or N-DGER results or IR causes an invention “improvement ripple effect” which extends far downstream from the immediate production of the IR. The direct use of one or more of these IRs in an application of any kind, herein termed a zero order application, to produce zero order application results which are, relative to prior art produced zero order application results, significantly improved, is a practice of the invention. An example of a zero order application is the direct use of invention improved abundance and/or RNA and/or NAS values for one or more particular genes in a cell sample to produce improved data mining analysis zero order results. A further downstream invention improvement ripple effect is the direct use of one or more zero order application IRs in an application of any kind, herein termed a first order application, to produce first order application results which are, relative to prior art produced first order application results, significantly improved, and a further practice of the invention. An example of a first order application is the direct use of data mining analysis IRs in a systems biology analysis application of any kind to produce improved systems biology results. An even further downstream invention improvement ripple effect is the direct use of one or more first order application IRs in an application of any kind, herein termed a second order application, to produce second order application results which are relative to prior art produced second order application results, significantly improved, and a practice of the invention. An example of a second order application is the direct use of one or more systems biology analysis IRs in a second order application for the discovery of drug candidates. Higher order invention improvement ripple effects also occur. The direct use of one or more improved lower order application IRs in a higher order application produces higher order application results which, relative to prior art produced higher order application results, are significantly improved, and is a practice of the invention. One of skill in the art will recognize that the use of lower order application IRs in a higher order application of any kind which utilizes lower order application results, will result in improved higher order application results.

In some aspects, the practice of the present invention involves the following, but not all of the items need be present in all practices of the invention. (a) Conduct one or more gene expression or gene expression comparison assays. (b) Determine for each gene expression or gene expression comparison assay the particular gene RN IR results and/or the particular gene abundance IR results and/or particular gene NAS IR results and/or the particular gene other equivalent IR results, and/or the particular gene comparison NASR IR results and/or the particular gene comparison N-DGER IR results and/or the particular gene comparison other equivalent IR results, by using the methods and means described in reference 148 and U.S. application Ser. No. 11/421,961. (c) Directly use one or more of the above described gene expression IRs in a higher order application. Such higher order applications include all prior art known and unknown higher order applications. Such methods include, but are not limited to, the above described simple and complex gene expression result analysis methods, as well as all higher order application methods described in references 1-147. This includes all supervised and unsupervised gene expression result analysis methods, as well as those gene expression analysis methods used in conjunction with systems biology analyses. Such methods include, but are not limited to, the following prior art classification methods and any valid modifications of such methods. (i) Linear discriminant methods. (ii) Support vector machine methods. (iii) K-nearest neighbor method. (iv) Neural network methods. (v) Decision tree methods. (vi) Partially supervised analysis methods. (vii) Class discovery method. Such methods also include, but are not limited to, the various prior art forms of class discovery and time series analysis and their modifications. Further, such analysis methods include the following prior art cluster analyses and any modifications of such methods. (i) Hierarchical agglomerative clustering methods. (ii) Hierarchical divisive clustering methods. (iii) Non-hierarchical K-means methods. (iv) Self-organizing maps and trees methods. (v) Relationship between clustering and principle component analysis methods. (vi) Gene shaving methods. (vii) Clustering in discretised space methods. (viii) Graph based clustering methods. (ix) Bayesian or model based clustering and fuzzy clustering methods. (x) Clustering of genes and samples methods. The above list was obtained from reference 10. References 9 and 10 contain useful summaries of prior art gene expression results analysis methods of all kinds. References 1-13 are also useful and contain many prior art gene expression result analysis method descriptions, and references to their use. Prior art analysis methods and other higher order applications and their use are also referred to in many of the references 14-147. (d) Use of one or more of the above described improved gene expression result analysis results or other high order application results in an even higher order application. Such applications include all prior art known and unknown other higher order applications which directly or indirectly utilize gene expression analysis assay results and/or gene expression assay result analysis results. Such applications include, but are not limited to, the very broad applications described in the Field of the Invention section and those described in the cited references.

One of skill in the art will recognize that any use of gene expression assay results which are known to be improved (IRs) for any gene expression assay results analysis, rather than prior art produced gene expression assay results which are known to be incorrect or uninterpretable gene expression assay results, produces improved gene expression result analysis results, relative to such prior art produced gene expression result analysis results, and that any production of such improved gene expression result analysis results is a practice of the invention. One of skill in the art will also recognize that any use of IRs and improved gene expression result analysis results and improved results for any other higher order application, rather than prior art produced gene expression assay results and gene expression result analysis results which are known to be incorrect or uninterpretable, produces improved any other higher order application results, relative to such prior art produced any other higher order application results, and that any production of any such improved higher order application result is a practice of the invention.

Conclusion

For the purpose of explanation the foregoing discussions used specific nomenclature to provide a thorough understanding of the invention and its many embodiments. However, it will be apparent to one of skill in the art that this nomenclature and description are but one way to describe the invention and its mode of practice. Thus, the foregoing nomenclature and description are presented for the purpose of illustration and description, and they are not intended to be exhaustive or to limit the invention to the precise forms disclosed, and obviously many modifications and variations are possible as a result of the above teachings. The discussions presented were selected and described in order to best explain the present invention and its practical applications, and to thereby enable others skilled in the art to best practice the invention and various embodiments with various modifications, as are suited to the particular use contemplated.

All publications and patents or patent applications cited in this specification-are herein incorporated by reference as if each individual publication or patent application were specifically and individually indicated by reference. The citation of any publication for its disclosure prior to the filing date should not be construed as an admission that the present invention is not entitled to antedate such publication by virtue of prior invention.

For simplicity the terms RN, abundance, NAS, NASR, N-DGER, SGDS, DGDS, DGSS, and IR are utilized in the claims. Said terms are discussed extensively in the accompanying patent application and are defined in the glossary of the accompanying patent application.

### REFERENCES CITED

1) Boultwood et al, Molecular Analysis of Cancer. Methods in Molecular Medicine. Vol 68 (2002) Humana Press
2) Burczynski, An Introduction To Toxicogenomics. CRC Press (2003)
3) McLachlan et al, Analyzing Microarray Gene Expression Data. Wiley Interscience Series in Probability and Statistics (2004)
4) Hamadeh et al, Toxiogenomics, Principles and Application. Wiley-Liss (2004)
5) Brenner et al, Oncogenomics, Molecular Approaches to Cancer. Wiley-Liss (2004)
6) Chin et al, Methods in Genomic Neuroscience. Methods New Frontiers in Neuroscience. CRC Press (2001)
7) Beraniver et al, Genomics and Proteomics in Nutrition. Marcel Dekker (2004)
8) Zhou et al, Microbial Functional Genomics. Wiley-Liss (2004)
9) Speed, Statistical Analysis of Gene Expression Microarray Data. Chapman and Hall/CRC (2003)
10) Causton et al, A Beginners Guide. Microarray Gene Expression Data Analysis. Blackwell Pub. (2004)
11) Kohane et al, Microarrays for an Integrative Genomics. Bradford Book, MIT Press (2003)
12) Draghici, Data Analysis Tools For DNA Microarrays. Chapman and Hall/CRC (2003)
13) Amaratunga et al, Exploration and Analysis of DNA Microarray and Protein Array Data. Wiley Series in Probability and Statistics, Wiley Nescience (2004)
14) Redei, Encyclopedic Dictionary of Genetics, Genomics, and Proteomics, Second Ed. Wiley-Liss (1998)
15) Pe'er et al, Inferring Subnetworks from Perturbed Expression Profiles. Bioinformatics Suppl. 17:S215-S224 (2001)
16) Jansen, Studying Complex Biological Systems Using Multifactorial Perturbation. Nature Reviews Genetics. 4:145-151 (2003)
17) Kraft, A Family-based Test For Correlation Between Gene Expression and Trait Values. Am. J. Hum. Genet. 72:1323-1330 (2003)
18) Cui et al, Integrated Analysis of Microarray Data and Gene Function Information. OMICS 8(2):2004
19) Friedman et al, Using Bayesian Networks to Analyses Expression Data. J. Computat. Biol. 7(3,4):601-620 (2000)
20) Hatzimanikatis et al, Dynamical Analysis of Gene Networks Requires Both mRNA and Protein Expression Information. Metabolic Engineering 1:275-281 (1999)
21) Oleksiak et al, Variations In Gene Expression Within and Among Natural Populations. Nature Genetics. 32:261-266 (2002)
22) Kraft et al, The Genetics of Gene Expression and Gene Mapping. Trends Biotech. 21(9):377-378 (2003)
23) D'Haeseleer et al, Genetic Network Inference: From Co-expression Clustering To Reverse Engineering. Bioinform. 16(8):707-726 (2000)
24) Hastie et al, “Gene Shaving” As A Method For Identifying Distinct Sets Of Genes With Similar Expression Patterns. Genome Biology 1(2):0003.1-0003.21 (2000)
25) de la Fuente et al, Quantifying Gene Networks With Regulatory Strengths. Molecular Biology Reports. 29:73-77 (2002)
26) Kerr et al, Bootstrapping Cluster Analysis: Assessing The Reliability of Conclusions From Microarray Experiments. Proc. Nat. Acad. Sci. US. 98(16):8961-8965 (2001)
27) Brazhnik et al, Gene Networks: How To Put The Function In Genomics. Trends Biotech. 20(11):467-472 (2002)
28) Holter et al, Dynamic Modeling of Gene Expression Data. Proc. Natl. Acad. Sci. US. 98(4):1693-1698 (2001)
29) Stark et al, From The Top Down: Towards A Predictive Biology of Signaling Networks. Trends Biotech. 21(7):290-293 (2003)
30) Herrgard et al, Reconciling Gene Expression Data With Known Genome-Scale Regulatory Network Structures. Genome Research. 13:2423-2434 (2004)
31) Jiminez et al, Microarray Analysis of Orthologous Genes: Conservation of The Translational Machinery Across Species At The Sequence and Expression Level. Genome Biol. 4:R4 (Dec. 3, 2002)
32) Wu et al, Global Analysis of Nutrient Control of Gene Expression in S. Cerevisiae During Growth and Starvation. Proc. Nat. Acad. Sci. US. 101(9):3148-3153 (2004)
33) Frankel, The Top Genes: On The Distance From Transcript To Function In Yeast Glycolisis. Current Opinion In Microb. 6:198-201 (2003)
34) Ihmels et al, Principles of Transcriptional Control In The Metabolic Network of S. Cerevisiae. Nature Biotech. 22(1):86-92 (2004)

35) Shedden et al, Analysis of Cell-Cycle Gene Expression In S. Cerevisiae Using Microarrays and Multiple Synchronization Methods. Nuc. Acids Res. 30(13):2920-2922 (2002)
36) Cho et al, transcriptional Regulation and Function During The Human Cell Cycle. Nature Genetics. 27:48-54 (2001)
37) Laub et al, Global Analysis Of The Genetic Network Controlling A Bacterial Cell Cycle. Science. 290:2144-2148 (2000)
38) Cummings et al, Using DNA Microarrays To Study Host-Microbe Interactions. Emerging Infectious Diseases. 6(5):513-525 (2000)
39) Wang et al, Viral Discovery and Sequence Recovery Using DNA Microarrays. PLOS Biology. 1(2):257-260 (2003)
40) Tao et al, Functional Genomics: Expression Analysis of E. Coli Growing On Minimal and Rich Media. J. Bact. 181(20):6425-6440 (1999)
41) Ma et al, Collaborative Regulation of E. Coli Glutamate Dependent Acid Resistance By Two AraC-Like Regulators, Gad X and Gad W (YhiW). J. Bact. 184(24):7001-7012 (2002)
42) Chang et al, Gene Expression Profiling of E. Coli Growth Transitions: An Expanded Stringent Response Model. Molec. Micro. 45(2):289-306 (2002)
43) Conway et al, Microreview-Microarray Expression Profiling: Capturing A Genomewide Portrait Of The Transcriptome. Molec. Micro. 47(4):879-889 (2003)
44) Frieberg et al, Genomewide mRNA Profiling: Impact on Compound Evaluation and Target Identification In Anti-Bacterial Research. Targets. 1(1):20-29 (2002)
45) Systems Biology Introduction. Kitano, Systems Biology: A Brief Overview-Csete et al, A Genomic Regulatory Network For Development-Noble, Modeling From The Heart: From Genes To Cells To The Whole Organ. Science 295:1661-1682 (2002)
46) Henry, Systems Biology. Chem and Eng. News. 83(7):47-55 (2005)
47) Tamayo et al, Interpreting Patterns of Gene Expression With Self-Organizing Maps: Methods and Applications To Hematopoietic Differentiation. Proc. Nat. Acad. Sci. US. 96:2907-2912 (1999)
48) Slonim et al, From Patterns To Pathways: Gene Expression Data Analysis Comes Of Age. Nature Genetics Suppl. 32:502-508 (2002)
49) Mobbs et al, Mining Microarrays For Metabolic Meaning: Nutritional Regulation of Hypothalmic Gene Expression. Neurochemical Res. 29(6):1093-1103 (2004)
50) Jansen et al, Genetical Genomics: The Added Value From Segregation. Trends Genetics. 17(7):388-391 (2001)
51) King et al, Gene Expression Profile Analysis By DNA Microarrays. Promises and Pitfalls. JAMA. 286(18):2280-2288 (2001)
52) Morley et al, Genetic Analysis Of Genomewide Variation In Human Gene Expression. Nature. ldoi:10.1038 Nature 027971 (2004)
53) Sellheyer et al, DNA Microarrays: From Structural Genomics To Functional Genomics. The Applications Of Gene Chips To Dermatology and Dermotopathology. J. Acad. Derm. 51(5):681-692 (2004)
54) Helmberg et al, DNA Microarrays: Novel Techniques To Study Aging and Guide Gerontologic Medicine. Experimental Gerontology. 36:1189-1198 (2001)
55) Whitney et al, Individual and Variation In Gene Expression Patterns In Human Blood. Proc. Nat. Acad. Sci. US. 100(4):1896-1901 (2003)
56) Tusher et al, Significance Analysis of Microarrays Applied To The Ionizing Radiation Response. Proc. Nat. Acad. Sci. 98(9):5116-5121 (2001)
57) Kim et al, A Gene Expression Map For Caenorhabditis Elegans. Science. 293:2087-2092 (2001)
58) Hutton et al, Microarray and Comparative Genomics-Based Identification Of Genes And Gene Regulatory Regions Of The Mouse Immune System. BMC Genomics 5:82 (Oct. 25, 2004)
59) Hamalainen et al, Distinct Gene Expression Profiles Of Human Type 1 and 2 T Helper Cells. Genome Biology 2 (7):0022.1-0022.11 (2001)
60) Parisi et al, A Survey Of Ovary-, Testis-, And Soma-Biased Gene Expression In Drosophila Melanogaster Adults. Genome Biology 5:R40 (2004)
61) Blackshaw et al, Genomic Analysis Of Mouse Retinal Development. PLOS Biol. 2(9) (e247):1411-1431 (2004)
62) Miller et al, Multi-Tissue Gene Expression Analysis In A Mouse Model Of Thyroid Hormone Resistance. Genome Biol. 5:R31.1-R31.17 (2004)
63) Schwab et al, A Catalogue Of Gene Expression In The Developing Kidney. Kidney International. 64:1588-1604 (2003)
64) Shergill et al, Tissue Microarrays: A Current Medical Research Tool. Current Md. Res. Opinion 20(5):707-712 (2004)
65) Wu et al, Plant Gene Expression Profiling With DNA Microarrays. Plant Physiol. Biochem. 39:917-926 (2001)
66) Rao et al, Gene Expression Profiles Of Peripheral Blood Cells In Type 2 Diabetes And Nephropathy In Asian Indians. Genome Biology:5:P9. (2004). Deposited Research.
67) Papapanou et al, Gene Expression Signatures In Chronic And Aggressive Periodontitis: A Pilot Study. Eur. J. Oral Sci. 112:216-223 (2004)
68) Sonna et al, Exceptional Heat Injury And Gene Expression Changes: A DNA Microarray Analysis Study. J. Applied Physiol. 96:1943-1953 (2004)
69) Mimics, Microarrays In Brain Research: The Good, The Bad and The Ugly. Nature 2:444-447 (2001)
70) Dash et al, A Molecular Description Of Brain Trauma Pathophysiology Using Microarray Technology: An Overview. Neurochem. Res. 29(6):1275-1286 (2004)
71) Pierce et al, Combining Brain Imaging With Microarray: Isolating Molecules Underlying The Physiologic Disorders Of The Brain. Neurochem. Res. 29(6):1145-1152 (2004)
72) Pisarchik et al, A Novel Microarray To Evaluate Stress Related Genes In Skin: Effect of Ultraviolet Light Radiation. Gene. 341:199-207 (2004)
73) Bair et al, Semi-Supervised Methods To Predict Patient Survival From Gene Expression Data. PLOS Biology. 2(4):511-522 (2004)
74) Huang et al, Gene Expression Phenotypic Models That Predict The Activity Of Oncogenic Pathways. Nature Genetics. 34(2):226-230 (2003)
75) Khan et al, Classification And Diagnostic Prediction Of Cancers Using Gene Expression Profiling And Artificial Neural Networks. Nature Medicine. 7(6):673-679 (2001)
76) Yan et al, Small Changes In Expression Affect Predisposition To Tumorigensis. Nature Genetics. 30: (Dec. 17, 2001 online)
77) Ochs et al, Microarrays In Cancer: Research And Applications. Biotechniques Suppl. (March 2003)
78) Scherf et al, A Gene Expression Database For The Molecular Pharmacology Of Cancer. Nature Genetics. 24:236-244 (2000)
79) Chung et al, Molecular Portraits And The Family Tree Of Cancer. Nature Genetics Suppl. 32:533-540 (2002)
80) Los et al, Using mRNA Expression Profiling To Determine Anticancer Drug Efficacy. Cytometry. 47:66-71 (2002)
81) Staunton et al, Chemosensitivity Prediction By Transcriptional Profiling. Proc. Nat. Acad. Sci. US. 98(19):10787-10792 (2001)
82) Angrist, Breast Cancer: Integrating The Patient With Her Genome. Trends Biotech. 23(1):3-5 (2005)
83) van't Veer et al, Gene Expression Profiling Predicts Clinical Outcome Of Breast Cancer. Nature. 415:530-536 (2002)
84) Monni et al, Comprehensive Copy Number And Gene Expression Profiling Of The 17q23 Amplicon In Human Breast Cancer. Proc. Nati. Acad. Sci. US. 98(10):5711-5716 (2001)
85) Sorlie et al, Gene Expression Patterns Of Breast Carcinomas Distinguish Tumor Subclasses With Clinical Implications. Proc. Nat. Acad. Sci. US. 98(19):10869-10874 (2001)
86) Perou et al, Molecular Portraits Of Human Breast Tumors. Nature. 406:747-752 (2000)
87) Sgroi et al, In Vivo Gene Expression Profile Analysis Of Human Breast Cancer Prognosis. Cancer Res. 59:5656-5661 (1999)
88) Symmans et al, Total RNA Yield And Microarray Gene Expression Profiles From Fine-Needle Aspiration Biopsy And Core-Needle Biopsy Samples Of Breast Carcinoma. Cancer. 97(12):2960-2971 (2003)
89) Dhanasekaran et al, Delineation Of Prognostic Biomarkers In Prostate Cancer. Nature. 412:822-831 (2001)
90) Luo et al, Human Prostate Cancer And Benign Prostatic Hyperplasia: Molecular Dissection By Gene Expression Profiling. Cancer Res. 61:4683-4688 (2001)
91) Mousses et al, Clinical Validation Of Candidate Genes Associated With Prostate Cancer Progression In The CWR22 Model System Using Tissue Microarrays. Cancer Res. 62:1256-1260 (2002)
92) Stuart et al, In Silico Dissection Of Cell-Type-Associated Patterns Of Gene Expression In Prostate Cancer. Proc. Nat. Acad. Sci. US. 101(2):615-620 (2004)
93) Schlicht et al, Cross Species Global And Subset Gene Expression Profiling Identifies Genes Involved In Prostate Cancer Response To Selenium. BMC Genomics. 5:58 (Aug. 20, 2004)
94) Panelli et al, Gene Expression Profiling Of The Response Of Peripheral Blood Mononuclear Cells And Melanoma Metastases To Systemic IL-2 Administration. Genome Biol. 3(7):0035.1-0035.17 (2002)
95) Bittner et al, Molecular Classification Of Cutaneous Malignant Melanoma By Gene Expression Profiling. Nature. 46:536-540 (2000)
96) Allzadeh et al, Distinct Types Of Diffuse Large B-Cell Lymphoma Identified By Gene Expression Profiling. Nature. 403:503-511(2000)
97) Grimwade et al, Gene Expression Profiling In Acute Myeloid Leukemia. New. Eng. J. Med. 350(16):1676-1678 (2004)
98) Pomeroy et al, Prediction Of CNS Embryonal tumor Outcome Based On Gene Expression. Nature. 415:436-442 (2002)
99) Mischel et al, DNA Microarray Analysis Of Brain Cancer: Molecular Classification For Therapy. Nature Reviews Neuro. 5:782-792 (2004)
100) Takano et al, Gene Expression Profiles In Thyroid Carcinomas. British J. Cancer. 83(11):1495-1502 (2000)
101) Parmigiani et al, A Cross Study Comparison Of Gene Expression Studies For The Molecular Classification Of Lung Cancer. Clin. Cancer Res. 10:2922-2927 (2004)
102) Garbner et al, Diversity Of Gene Expression In Adenocarcinoma Of The Lung. Proc. Nat. Acad. Sci. US. 98 (24):13784-13789 (2001)
103) Bhattacharjee et al, Classification Of Human Lung Carcinomas By mRNA Expression Profiling Reveals Distinct Adenocarcinoma Subclasses. Proc. Nat. Acad. Sci. US. 98(24):13790-13795 (2001)
104) Welsh et al, Analysis of Gene Expression Profiles In Normal And Neoplastic Ovarian Tissue Samples Identifies Candidate Molecular Markers Of Epithelial Ovarian Cancer. Proc. Nat. Acad. Sci. 98(3):1176-1181 (2001)
105) Shridhar et al, Genetic Analysis Of Early vs. Late Stage Ovarian Tumors. Cancer Res. 61:5895-5904 (2004)
106) Muro et al, Identification Of Expressed Genes Linked To Malignancy Of Human Colorectal Carcinoma By Parametric Clustering Of Quantitative Expression Data. Genome Biol. 4: R21 (2003)
107) Hedge et al, Identification Of Tumor Markers In Models Of Human Colorectal Cancer Using A 19, 200 Element cDNA Microarray. Cancer Res. 61:7792-797 (2001)
108) Chang et al, Gene Expression Signature Of Fibroblast Serum Response Predicts Human Cancer Progression: Similarities Between Tumors and Wounds. PLOS Biol. 2(2):206-214 (2004)
109) Su et al, Molecular Classification Of Human Carcinomas By Use Of Gene Expression Signatures. Cancer Res. 61:7388-7393(2001)
110) Whitfield et al, Identification Of Genes Periodically Expressed In The Human Cell Cycle And Their Expression In Tumors. Molec. Biol. of Cell. 13:1977-2000 (2002)
111) Yap et al, Classification Between Normal And Tumor Tissues Bases On The Pair-Wise Gene Expression Ratio. BMC Cancer. 4:72 (Oct. 7, 2004)
112) Gieseg et al, The Influence Of Tumor Size And Environment On Gene Expression In Commonly Used Human Tumor Lines. BMC Cancer. 4:35 (Jul. 15, 2004)
113) Russo et al, Advantages And Limitations Of Microarray Technology In Human Cancer. Oncogene. 22:6497-6507 (2003)
114) Warrington et al, Microarrays And Cancer Research. Biotechniques Press, Eaton Pub. (2002)
115) Thomas et al, Identification Of Toxicology Predictive Gene Sets Using cDNA Microarrays. Molec. Pharm. 60(6):1189-1194 (2001)
116) Kriete et al, Combined Histomorphometric And Gene Expression Profiling Applied To Toxicology. Genome Biol. 4: R32 (Apr. 30, 2003)
117) Karyala et al, Different Global Gene Expression Profiles In Benzo(a) Pyrene- And Dioxin-Treated Vascular Smooth Muscle Cells Of AHR-Knockout And Wild-Type Mice. Cardiovascular Toxic 4:47-74 (2004)
118) Young et al, Analysis of Gene Expression In Carbon Tetra-Chloride Treated Rat Livers Using A Novel Bioarray Technology. Pharmacogenomics J. 3:41-52 (2003)
119) Terasaka et al, Using A Custom DNA Microarray For Expression Profiling Of The Estrogen-Responsive Genes To Evaluate Estrogen Activity Among Natural Estrogens And Industrial Chemicals. Environ. Health Perspectives. 112(7):773-781 (2004)
120) Kudoh et al, Monitoring The Expression Profiles Of Doxorubicin-Induced and Doxorubicin-Resistant Cancer Cells By cDNA Microarray. Cancer Res. 60:4161-4166 (2000)
121) Boess et al, Gene Expression In Two Hepatic Cell Lines, Cultured Primary Hepatocytes, And liver Slices Compared To The In Vivo Liver Gene Expression In Rats: Possible Implications For Toxicogenomics Use Of In Vivo Systems. Toxicological Sciences. 73:386-402 (2003)
122) Ulrich et al, Toxicogenomics And Drug Discovery: Will New Technologies Help Us Produce Better Drugs? Nature Reviews Drug Discovery. 1:84-88 (2001)
123) Issa, Ethical Perspectives On Pharmacogenomics Profiling In The Drug Development Process. Nature Reviews Drug Discovery. 1:300-308 (2002)
124) Searls, Data Integration: Challenges For Drug Discovery. Nature Reviews Drug Discovery. 4:45-58 (2005)
125) Browne et al, Predictive Chemoinformatics, Application To The Pharmaceutical Industry. Drug Discovery World: Pages 71-77 (Fall 2002)
126) Gerhold et al, Better Therapeutics Through Microarrays. Nature Genetics Suppl. 32:547-552 (2002)
127) Basik et al, Integration Of Genomic Technologies For Accelerated Cancer Drug Development. Biotechniques. 35:580-593 (2003)
128) Thayer, Biomarkers Emerge. Chem and Eng. News. Pages 33-37 (Jul. 28, 2003)
129) Thayer, The Genomics Evolution. Chem and Eng. News. Pages 17-26 (Dec. 8, 2003)
130) Marton et al, Drug Target Validation And Identification Of Secondary Drug Target Effects Using DNA Microarrays. Nature Medicine. 4(11):1293-1301 (1998)
131) Dean et al, Industrial-Scale, Genomics-Based Drug Design And Discovery. Trends Guide To Genetic Variation And Genomic Medicine. Pages 537-547 (March 2002)
132) Sauter et al, Tissue Microarrays In Drug Discovery. Nature Reviews Drug Discovery. 2:962-972 (2003)
133) Broxton et al, The Integration Of Microarray Information In The Drug Development Process. Current Opinion In Biotech. 9:643-649 (1998)
134) Young, Biomedical Discovery With DNA Arrays. Cell. 102:9-15 (2000)
135) Crowther, Applications Of Microarrays In The Pharmaceutical Industry: And Spang, Diagnostic Signatures From Microarrays. A Bioinformatics Concept For Personalized Medicine. Drug Discovery Today Suppl. 9(24):518-521 and 532-536 (2004)
136) Veber et al, The New Partnership Of Genomics And Chemistry For Accelerated Drug Development. Current Opinion In Chem. Biol. 1:151-156 (1997)
137) Dohrmann, Target Discovery In Metabolic Disease. Drug Discovery Today. 9(18):785-794 (2004)
138) Grigoryev et al, Orthologous Gene Expression Profiling In Multi-Species Models: Search For Candidate Genes. Genome Biol. 5: R34 (Apr. 27, 2004)
139) Somogyi et al, The Dynamics Of Molecular Networks: Application To Therapeutic Discover. Drug Development Today. 6(24):1267-1277 (200 1)
140) Butte et al, Discovering Functional Relationships Between RNA Expression And Chemotherapeutic Susceptibility Using Relevance Networks. Proc. Nat. Acad. Sci. US. 97(22):12182-12186 (2000)
141) Nevins et al, Towards Integrated Clinico-Genomic Models For Personalized Medicine: Combining Gene Expression Signatures And Clinical Factors In Breast Cancer Outcomes Prediction. Human Mol. Genetics. 12 (Review Issue 2): R153-R157 (2003)
142) Pittman et al, Integrated Modeling Of Clinical And Gene Expression Information For Personalized Prediction Of Disease Outcomes. Proc. Natl. Acad. Sci. US. 101(22):8431-8436 (2004)
143) Scarselli et al, The Impact Of Genomics On Vaccine Design. Trends Biotech. 23(2):84-91 (2005)
144) DeFilippis et al, Functional Genomics In Virology And Antiviral Drug Discovery. Trends Biotech. 21(10):452-457 (2003)
145) Petricoin et al, Medical Applications Of Microarray Technologies: A Regulatory Science Perspective. Nature Genetics Suppl. 32:474-479 (2002)
146) Jackson et al, Expression Profiling Reveals Off-Target Gene Regulation By RNAi. Nature Biotech. 21(6):635-637 (2003)
147) Lim et al, Microarray Analysis Shows That Some microRNAs Downregulate Large Numbers Of Target mRNAs. Nature. 433:769-773 (2005)
148) Kohne, U.S. Provisional Pat. No.: 60/687,526 Title: Method for Producing Improved Gene Expression Analysis and Gene Expression Analysis Comparison Results.

### EXAMPLES

### Example 1

### Methods Utilizing Gene Expression Profiles

As described above, the present methods for providing improved results in application involving or utilizing gene expression results and profiles can be applied in many different general and specific applications. An exemplary list of U.S. patents and published patent applications describing inventions to which the present invention is applicable is provided below. Thus, inventions involving gene expression determinations and profiles described in these patents and patent applications provide illustrative embodiments for use of the present invention. The listed patents and patent applications provide both particular methods and applications, and illustrate broader applications for the present invention. Each of the listed patents and patent applications are incorporated herein by reference in its entirety.

Even though the list includes a large number of documents, it should be recognized that these are merely illustrative of a much larger set of applications for this invention. In these applications, as well as in the broader set including applications not covered by the list, the use of improved gene expression results according to the present invention provides improved gene expression profiles and improved applications resulting from use of such improved results and profiles.

The inventions described in the following patent documents and the disclosures therein are representative of methods and compositions used by those of skill in the art, as well as being illustrative of methods which can be improved by the present invention and therefore represent embodiments of this invention.

Documents concerning gene expression profiles
Patents

| PAT. NO. | Title |
|---|---|
| 7,054,755 | Interactive correlation of compound information and genomic information |
| 7,049,102 | Multi-gene expression profile |
| 7,049,072 | Gene expression analysis of pluri-differentiated mesenchymal progenitor cells |
| | and methods for diagnosing a leukemic disease state |
| 7,031,843 | Computer methods and systems for displaying information relating to gene |
| | expression data |
| 7,011,947 | MLL translocations specify a distinct gene expression profile, distinguishing a |
| | unique leukemia |
| 6,998,234 | Methods for cancer prognosis and diagnosis relating to tumor vascular |
| | endothelial cells |
| 6,949,342 | Prostate cancer diagnosis and outcome prediction by expression analysis |
| 6,913,882 | Methods of screening for B cell activity modulators |
| 6,884,578 | Genes differentially expressed in secretory versus proliferative endometrium |
| 6,876,930 | Automated pathway recognition system |
| 6,774,120 | Methods of inducing regulated pancreatic hormone production in non- |
| | pancreatic islet tissues |
| 6,686,156 | Methods and compositions for transcription-based nucleic acid amplification |
| 6,607,885 | Method for high-density microarray medicated gene expression profiling |
| 6,569,624 | Identification of genetic markers of biological age and metabolism |
| 6,524,787 | Diagnostics and therapy based on vascular mimicry |
| 6,511,806 | Methods for cancer prognosis and diagnosis |
| 6,470,277 | Techniques for facilitating identification of candidate genes |
| 6,465,215 | Identification of cells for transplantation |
| 6,406,853 | Interventions to mimic the effects of calorie restriction |
| 6,365,352 | Process to study changes in gene expression in granulocytic cells |
| 6,291,170 | Multi-genes expression profile |
| 6,203,987 | Methods for using co-regulated genesets to enhance detection and classification |
| | of gene expression patterns |

Published Patent Applications

| Publ. No. | Title |
|---|---|
| 20060104952 | Genomic barrier to viral disease |
| 20060084083 | Method to generate or determine nucleic acid tags corresponding to the |
| | terminal ends of DNA molecules using sequences analysis of gene |
| | expression (terminal SAGE) |
| 20060078900 | Molecular toxicology modeling |
| 20060074566 | Methods and systems for gene expression array analysis |
| 20060063156 | Outcome prediction and risk classification in childhood leukemia |
| 20060057630 | MLL translocations specify a distinct gene expression profile, |
| | distinguishing a unique leukemia |
| 20060046259 | Differential expression of molecules associated with acute stroke |
| 20060029971 | Prostate cancer diagnosis and outcome prediction by expression analysis |
| 20060024734 | MLL translocations specify a distinct gene expression profile, |
| | distinguishing a unique leukemia |
| 20060015952 | Screening assays and methods of tumor treatment |
| 20060009410 | Effects of apolipoprotein B inhibition on gene expression profiles in |
| | animals |
| 20060008838 | Prostate cancer diagnosis and outcome prediction by expression analysis |
| 20060008803 | Identification of tissue/cell specific marker genes and use thereof |
| 20050287570 | Probe arrays for expression profiling of rat genes |
| 20050282207 | Method of stress evaluation |
| 20050266438 | Genetic networks regulated by attenuated GH/IGF1 signaling and caloric |
| | restriction |
| 20050260586 | Method and compositions for the diagnosis and treatment of non-small cell |
| | lung cancer using gene expression profiles |
| 20050227983 | Triazine compounds and their analogs, compositions, and methods |
| 20050221310 | Methods for enhancing gene expression analysis |
| 20050208512 | Determining the chemosensitivity of cells to cytotoxic agents |
| 20050208493 | Ordering genes by analysis of expression kinetics |
| 20050191272 | Methods for producing Ex vivo models for inflammatory disease and uses |
| | thereof |
| 20050181424 | Method for preparing gene expression profile |
| 20050181377 | Targeted cancer therapy |
| 20050171338 | Mammalian tribbles signaling pathways and methods and reagents related |
| | thereto |
| 20050170375 | Methods for enhancing gene expression analysis |
| 20050170373 | Expression profiling using microarrays |
| 20050170351 | Materials and methods relating to cancer diagnosis |
| 20050164196 | Methods to predict patient responsiveness to tyrosine kinase inhibitors |
| 20050148506 | Novel method of modulating bone-related activity |
| 20050142119 | Human mesenchymal progenitor cell |
| 20050137805 | Gene expression profiles that identify genetically elite ungulate mammals |
| 20050123966 | Diagnostic and prognostic methods and compositions for seizure- and |
| | plasticity-related disorders |
| 20050113341 | Medical devices employing triazine compounds and compositions thereof |
| 20050100929 | Methods and systems for gene expression array analysis |
| 20050095634 | qRT-PCR assay system for gene expression profiling |
| 20050089899 | Identification of an ERBB2 gene expression signature in breast cancers |
| 20050084896 | Process to study changes in gene expression in granulocytic cells |
| 20050084880 | Systems and methods for diagnosing & treating psychological and |
| | behavioral conditions |
| 20050074793 | Metastatic colorectal cancer signatures |
| 20050069886 | Prostate cancer genes |
| 20050060102 | Interactive correlation of compound information and genomic information |
| 20050059147 | Human mesenchymal progenitor cell |
| 20050059072 | Selective modulation of TLR gene expression |
| 20050055166 | Nonlinear modeling of gene networks from time series gene expression |
| | data |
| 20050037430 | Methods and uses for protein breakdown products |
| 20050032096 | Methods for predicting drug sensitivity in patients afflicted with |
| | hypertension |
| 20050027460 | Method, program product and apparatus for discovering functionally |
| | similar gene expression profiles |
| 20050003422 | Methods for assessing and treating cancer |
| 20040265230 | Compositions and methods for diagnosing and treating colon cancers |
| 20040248135 | Methods for detemining multiple effects of drugs that modulate function of |
| | transcription regulatory proteins |
| 20040248116 | Prostate cancer expression profiles |
| 20040241706 | Highly specific modulators of GTPases for target validation |
| 20040236516 | Bioinformatics based system for assessing a condition of a performance |
| | animal by analysing nucleic acid expression |
| 20040225445 | Systems and methods for characterizing a biological condition or agent |
| | using selected gene expression profiles |
| 20040224950 | Methods and compositions of novel triazine compounds |
| 20040214203 | Genes related to sensitivity and resistance to chemotherapeutic drug |
| | treatment |
| 20040209882 | Methods and compositions of novel triazine compounds |
| 20040209881 | Methods and compositions of novel triazine compounds |
| 20040209880 | Methods and compositions of novel triazine compounds |
| 20040204420 | Compounds for modulating RNA interference |
| 20040203019 | Methods and compositions for transcription-based nucleic acid |
| | amplification |
| 20040191779 | Statistical analysis of regulatory factor binding sites of differentially |
| | expressed genes |
| 20040175733 | Multiplex amplification of polynucleotides |
| 20040157229 | Methods of profiling gene expression, protein or metabolite levels |
| 20040146922 | Cellular arrays for the identification of altered gene expression |
| 20040146894 | Methods of diagnosing and treating stress urinary incontinence |
| 20040142373 | Cellular arrays for the identification of altered gene expression |
| 20040133352 | Identification, monitoring and treatment of disease and characterization of |
| | biological condition using gene expression profiles |
| 20040115801 | Model epithelial cell cultures |
| 20040110792 | Methods for assessing and treating leukemia |
| 20040101818 | Gene expression profiles associated with osteoblast differentiation |
| 20040096878 | Methods for isolating and characterizing endogenous mRNA-protein |
| | (mRNP) complexes |
| 20040077648 | Methods and compositions of novel triazine compounds |
| 20040072305 | Gene expression profiling from FFPE samples |
| 20040072170 | Novel target genes for diseases of the heart |
| 20040072160 | Molecular toxicology modeling |
| 20040067583 | Methods for immortalizing cells |
| 20040067500 | Compositions and methods relating to the peroxisomal proliferator |
| | activated receptor-alpha mediated pathway |
| 20040053294 | Methods for identifying target genes, gene expression profiles, and |
| | biochemical/signal transduction pathways associated with specific patterns |
| | of hemodynamic shear stress and atherogenesis and uses thereof |
| 20040038201 | Diagnostic and therapeutic applications for biomarkers of infection |
| 20040033502 | Gene expression profiles in esophageal tissue |
| 20040031065 | Neuronal activation in a transgenic model |
| 20040018527 | Differential patterns of gene expression that predict for docetaxel |
| | chemosensitivity and chemo resistance |
| 20040014040 | Cardiotoxin molecular toxicology modeling |
| 20040009495 | Methods and products related to drug screening using gene expression |
| | patterns |
| 20040009494 | Novel methods of diagnosis of angiogenesis and other conditions, |
| | compositions, and the methods of screening for modulators |
| 20040009489 | Classification of lung carcinomas using gene expression analysis |
| 20040009485 | Cellular arrays for the identification of altered gene expression |
| 20040005625 | Method of analyzing expression of gene |
| 20040005614 | Methods for fragmentation, labeling and immobilization of nucleic acids |
| 20040002094 | Method for high-density microarray mediated gene expression profiling |
| 20030235830 | Methods for isolating and characterizing endogenous mRNA-protein |
| | (mRNP) complexes |
| 20030229455 | Systems and methods for characterizing a biological condition or agent |
| | using precision gene expression profiles |
| 20030224360 | Interventions to mimic the effects of calorie restriction |
| 20030219771 | Identification, monitoring and treatment of disease and characterization of |
| | biological condition using gene expression profiles |
| 20030219764 | Biological discovery using gene regulatory networks generated from |
| | multiple-disruption expression libraries |
| 20030219736 | Cellular arrays for the identification of altered gene expression |
| 20030218634 | System and methods for visualizing diverse biological relationships |
| 20030215835 | Differentially-regulated prostate cancer genes |
| 20030207270 | Phytomics: a genomic-based approach to herbal compositions |
| 20030203483 | Human mesenchymal progenitor cell |
| 20030194701 | Diffuse large cell lymphoma diagnosis and outcome prediction by |
| | expression analysis |
| 20030191048 | Gene expression profile for KSHV infection and methods for treating same |
| 20030182302 | System and method for identifying networks of ternary relationships in |
| | complex data systems |
| 20030170625 | Detection of differential gene expressions |
| 20030165910 | Microassay for serial analysis of gene expression and applications thereof |
| 20030157526 | Identification of genetic markers of biological age and metabolism |
| 20030157073 | Methods for pretreating a subject with apoptotic cells |
| 20030152980 | Prostate cancer diagnosis and outcome prediction by expression analysis |
| 20030152550 | Dendritic cells and the uses thereof in screening cellular targets and |
| | potential drugs |
| 20030148295 | Expression profiles and methods of use |
| 20030139466 | Methods for pretreating a subject with extracorporeal photopheresis |
| 20030134776 | Methods for predicting drug sensitivity in patients afflicted with an |
| | inflammatory disease |
| 20030134324 | Identifying drugs for and diagnosis of Benign Prostatic Hyperplasia using |
| | gene expression profiles |
| 20030134320 | Method system and computer program product for quality assurance in |
| | detecting biochemical markers |
| 20030134300 | MLL translocations specify a distinct gene expression profile, |
| | distinguishing a unique leukemia |
| 20030134280 | Identifying drugs for and diagnosis of benign prostatic hyperplasia using |
| | gene expression profiles |
| 20030124540 | Interventions to mimic the effects of calorie restriction |
| 20030113831 | Methods for predicting drug sensitivity in patients afflicted with an |
| | inflammatory disease |
| 20030104394 | Method and system for gene expression profiling analysis utilizing |
| | frequency domain transformation |
| 20030096782 | Expression profiling in the intact human heart |
| 20030096290 | Methods for cancer prognosis and diagnosis |
| 20030096261 | Methods for cancer prognosis and diagnosis relating to tumor vascular |
| | endothelial cells |
| 20030096234 | Canine toxicity genes |
| 20030093226 | Methods for the identification of reporter and target molecules using |
| | comprehensive gene expression profiles |
| 20030087251 | Methods and compositions for amplification of RNA sequences |
| 20030082512 | Process to study changes in gene expression in granulocytic cells |
| 20030054394 | Techniques for facilitating identification of candidate genes |
| 20030054367 | Method for correlating gene expression profiles with protein expression |
| | profiles |
| 20030049598 | Methods for producing ex vivo models for inflammatory disease and uses |
| | thereof |
| 20030036522 | Identification of cells for transplantation |
| 20030036079 | Gene expression alterations underlying the retardation of aging by caloric |
| | restriction in mammals |
| 20030033126 | Modeling biological systems |
| 20030022318 | Method for thermocycling amplification of nucleic acid sequences and the |
| | generation of related peptides thereof |
| 20030003084 | Human mesenchymal progenitor cell |
| 20020177157 | Pairs of nucleic acid probes with interactive signaling moieties and nucleic |
| | acid probes with enhanced hybridization efficiency and specificity |
| 20020174096 | Interactive correlation of compound information and genomic information |
| 20020173461 | Methods for enhancing the efficacy of cancer therapy |
| 20020168664 | Automated pathway recognition system |
| 20020164628 | Methods and compositions for amplification of RNA sequences |
| 20020155480 | Brain tumor diagnosis and outcome prediction |
| 20020155463 | Prostate polynucleotides and uses |
| 20020155422 | Methods for analyzing dynamic changes in cellular informatics and uses |
| | therefor |
| 20020142981 | Gene expression profiles in liver cancer |
| 20020058270 | Methods and compositions for transcription-based nucleic acid |
| | amplification |
| 20020007051 | Use of multiple recombination sites with unique specificity in |
| | recombinational cloning |
| 20020004211 | Methods for isolating and characterizing endogenous mRNA-protein |
| | (mRNP) complexes |
| 20010044104 | Genes differentially expressed in secretory versus proliferative |
| | endometrium |

Documents concerning gene expression profiling
Published Patent Applications

| Publ. No. | Title |
|---|---|
| 20060100788 | Collections of matched biological reagents and methods for identifying |
| | matched reagents |
| 20060078925 | Novel microarray techniques for nucleic acid expression analyses |
| 20050158756 | Identification of a gene expression profile that differentiates ischemic and |
| | nonischemic cardiomyopathy |
| 20050112630 | Diagnosis, prognosis and identification of potential therapeutic targets of |
| | multiple myeloma based on gene expression profiling |
| 20040260721 | Methods and systems for creation of a coherence database |
| 20040229245 | Methods and algorithms for performing quality control during gene |
| | expression profiling on DNA microarray technology |
| 20040220125 | Biosynthetic platform for cardioprotective gene expression using immature |
| | heart tissue |
| 20040162679 | Method for predicting gene potential and cell commitment |
| 20040117128 | Methods, computer software products and systems for gene expression |
| | cluster analysis |
| 20040009523 | Diagnosis, prognosis and identification of potential therapeutic targets of |
| | multiple myeloma based on gene expression profiling |
| 20030232364 | Diagnosis, prognosis and identification of potential therapeutic targets of |
| | multiple myeloma based on gene expression profiling |
| 20030108872 | Genomics-assisted rapid identification of targets |
| 20030096309 | Screening system for identifying drug-drug interactions and methods of use |
| | thereof |
| 20030022157 | Methods of producing a library and methods of selecting polynucleotides of |
| | interest |

Documents concerning gene profiling
Published Patent Applications

| PUBL. NO. | Title |
|---|---|
| 20040229817 | Inhibitors of Hepatitis C virus, compositions |
| | and treatments using the same |
| 20040115671 | Gene expression profiling of endothelium |
| | in alzheimer's disease |

Documents concerning gene profile
Patents

| PAT. NO. | Title |
|---|---|
| 7,049,102 | Multi-gene expression profile |
| 6,426,185 | Method of compiling a functional gene |
| | profile in a plant by transfecting a nucleic acid sequence |
| | of a donor plant into a different host plant in an anti- |
| | sense orientation |

Published Patent Applications

| Publ. No. | Title |
|---|---|
| 20040088757 | Cytoplasmic gene inhibition or gene expression in transfected plants by |
| | tobraviral vector |
| 20030182302 | System and method for identifying networks of ternary relationships in |
| | complex data systems |
| 20030167512 | Method of determining the presence of a trait in a plant by transfecting a |
| | nucleic acid sequence of a donor plant into a different host plant in a positive |
| | orientation |
| 20030157526 | Identification of genetic markers of biological age and metabolism |
| 20030104394 | Method and system for gene expression profiling analysis utilizing frequency |
| | domain transformation |
| 20020192681 | Response of dendritic cells to a diverse set of pathogens |
| 20020165370 | Cytoplasmic gene inhibition or gene expression in transfected plants by a |
| | tobraviral vector |
| 20020042054 | Vigilance nucleic acids and related diagnostic, screening and therapeutic |
| | methods |

Documents concerning expression pattern
Patents

| PAT. NO. | Title |
|---|---|
| 7,041,449 | Methods of screening for compounds that inhibit expression of biomarker |
| | sequences differentially expressed with age in mice |
| 7,031,847 | Method and apparatus for displaying gene expression patterns |
| 6,916,603 | Methods of using agents that modulate bone formation and inhibit |
| | adipogenesis |
| 6,876,930 | Automated pathway recognition system |
| 6,862,726 | Light intensity simulation method, program product, and designing method of |
| | photomask |
| 6,783,929 | Biological sample component purification and differential display |
| 6,764,674 | Adenovirus E1B shuttle vectors |
| 6,733,969 | Expression monitoring for gene function identification |
| 6,651,097 | Learning support method, system and computer readable medium storing |
| | learning support program |
| 6,647,341 | Methods for classifying samples and ascertaining previously unknown classes |
| 6,436,642 | Method of classifying a thyroid carcinoma using differential gene expression |
| 6,335,170 | Gene expression in bladder tumors |
| 6,326,146 | Method of determining multiple mRNAs in dying cells |
| 6,303,301 | Expression monitoring for gene function identification |
| 6,232,066 | High throughput assay system |
| 6,197,506 | Method of detecting nucleic acids |
| 6,007,993 | In vitro test for embryotoxic and teratogenic agents using differentiation- |
| | dependent reporter expression in pluripotent rodent embryonic cells |
| 5,958,688 | Characterization of mRNA patterns in neurites and single cells for medical |
| | diagnosis and therapeutics |
| 5,932,478 | Human colon carcinoma cell lines showing stable HSP72 expression |
| 5,802,262 | Method and apparatus for diagnosing lexical errors |
| 5,665,589 | Human liver epithelial cell lines |
| 5,481,626 | Numerical expression reognizing apparatus |

Published Patent Applications

| Publ. No. | Title |
|---|---|
| 20060122792 | Method and system for predicting gene pathway using gene expression |
| | pattern data and protein interaction data |
| 20060117399 | Animal model for human lymphomas |
| 20060106832 | Method and system for implementing an enhanced database |
| 20060104952 | Genomic barrier to viral disease |
| 20060099628 | Diagnostic assay for rickettsia prowazekii disease by detection of |
| | responsive gene expression |
| 20060094018 | Discovery and a method for the early detection of pancreatic cancer and |
| | other disease conditions |
| 20060088876 | Method for the early detection of breast cancer, lung cancer, pancreatic |
| | cancer and colon polyps, growths and cancers as well as other |
| | gastrointestinal disease conditions and the preoperative and postoperative |
| | monitoring of transplanted organs from the donor and in the recipient and |
| | their associated conditions related and unrelated to the organ transplantation |
| 20060084075 | Program for analysis of the time-series data obtained by DNA array |
| | method, a method for analysis of the time-series data obtained by DNA |
| | array method, and a device for analysis of the time-series data obtained by |
| | DNA array method |
| 20060073496 | Methods of identifying patients at risk of developing encephalitis following |
| | immunotherapy for Alzheimer's disease |
| 20060063157 | Colorectal cancer prognostics |
| 20060046249 | Identification of polynucleotides and polypetide for predicting activity of |
| | compounds that interact with protein tyrosine kinase and or protein tyrosine |
| | kinase pathways |
| 20060037088 | Gene expression levels as predictors of chemoradiation response of cancer |
| 20060029960 | Method for the early detection of pancreatic cancer and other |
| | gastrointestinal disease conditions |
| 20060029944 | Identification of polynucleotides for predicting activity of compounds that |
| | interact with and/or modulate protein tyrosine kinases and/or protein |
| | tyrosine kinase pathways in breast cells |
| 20060019284 | Identification of polynucleotides for predicting activity of compounds that |
| | interact with and/or modulate protein tyrosine kinases and/or protein |
| | tyrosine kinase pathways in lung cancer cells |
| 20060014301 | Antibody-based system for detection of differential protein expression |
| | patterns |
| 20060004529 | Method, computer program product with program code segments and |
| | computer program product for analysis of a regulatory genetic network of a |
| | cell |
| 20050266562 | Myeloid cell promoter and constructs containing same |
| 20050260629 | Differential drug sensitivity |
| 20050260572 | Method of predicting cancer |
| 20050246795 | Root-specific expansin gene regulating root growth and obstacle-touching |
| | stress resistance in the plant |
| 20050240352 | Online procurement of biologically related products/services using |
| | interactive context searching of biological information |
| 20050208499 | Markers for diagnosing and treating breast and ovarian cancer |
| 20050188294 | Systems, tools and methods for constructing interactive biological diagrams |
| 20050186577 | Breast cancer prognostics |
| 20050181399 | Methods for enhanced detection & analysis of differentially expressed |
| | genes using gene chip microarrays |
| 20050176022 | Vertebrate telomerase genes and proteins and uses thereof |
| 20050171338 | Mammalian tribbles signaling pathways and methods and reagents related |
| | thereto |
| 20050164244 | Methods of determining juvenile arthritis classification |
| 20050158733 | EGR genes as targets for the diagnosis and treatment of schizophrenia |
| 20050147978 | Method for quantitative determination of multi-drug resistance in tumors |
| 20050136452 | Methods for monitoring expression of polymorphic alleles |
| 20050130212 | Method, computer program having program code means and computer |
| | program product for analyzing a regulatory genetic network of a cell |
| 20050130189 | Compositions and methods for treating and diagnosing irritable bowel |
| | syndrome |
| 20050130187 | Method for identifying relevant groups of genes using gene expression |
| | profiles |
| 20050125161 | Differentially-expressed conifer cDNAs, and their use in improving somatic |
| | embryogenesis |
| 20050095600 | Methods of generating gene-specific probes for nucleic acid array detection |
| 20050089895 | Compositions and methods for prognosis and therapy of liver cancer |
| 20050084872 | Methods for determining whether an agent possesses a defined biological |
| | activity |
| 20050074793 | Metastatic colorectal cancer signatures |
| 20050069893 | Diagnostic method for glaucoma |
| 20050059147 | Human mesenchymal progenitor cell |
| 20050048526 | Colorectal cancer prognostics |
| 20050048494 | Colorectal cancer prognostics |
| 20050003410 | Allele-specific expression patterns |
| 20040248116 | Prostate cancer expression profiles |
| 20040241751 | Arrays of protein-capture agents and methods of use thereof |
| 20040234979 | Differentiall-expressed and up-regulated polynucleotides and polypeptides |
| | in breast cancer |
| 20040229224 | Allele-specific expression patterns |
| 20040221332 | Plant growth regulating genes, proteins and uses thereof |
| 20040219575 | Methods and compositions for the diagnosis, prognosis, and treatment of |
| | cancer |
| 20040214203 | Genes related to sensitivity and resistance to chemotherapeutic drug |
| | treatment |
| 20040180038 | Effectors of innate immunity determination |
| 20040162679 | Method for predicting gene potential and cell commitment |
| 20040152168 | Neurogenin 3 promoter |
| 20040143854 | Method for generating a genetically modified organism |
| 20040122673 | Method of and apparatus for managing dialog between user and agent |
| 20040121407 | Regulation of the growth hormone/IGF-1 axis |
| 20040116330 | Preventive/therapeutic method for cancer |
| 20040111220 | Methods of decomposing complex data |
| 20040106555 | Neurogenin3 and production of pancreatic islet cells |
| 20040106132 | Identification of polynucleotides for predicting activity of compounds that |
| | interact with and/or modulate protein tyrosine kinases and/or protein |
| | tyrosine kinase pathways in breast cells |
| 20040086855 | Method for screening genes expressing at desired part |
| 20040081618 | Method of screening physiologically active substance |
| 20040067500 | Compositions and methods relating to the peroxisomal proliferator |
| | activated receptor-alpha mediated pathway |
| 20040058376 | Expression monitoring for gene function identification |
| 20040058340 | Diagnosis and prognosis of breast cancer patients |
| 20040058325 | Gene expression in biological conditions |
| 20040053340 | Protein arrays |
| 20040038917 | Gene expression in biological conditions |
| 20040038207 | Gene expression in bladder tumors |
| 20040014095 | Targets, methods, and reagents for diagnosis and treatment of schizophrenia |
| 20040005644 | Method and composition for detection and treatment of breast cancer |
| 20040001803 | Effectors of innate immunity determination |
| 20030232353 | Methods of analysis of allelic imbalance |
| 20030225526 | Molecular cancer diagnosis using tumor gene expression signature |
| 20030224374 | Diagnosis and prognosis of breast cancer patients |
| 20030194711 | System and method for analyzing gene expression data |
| 20030170720 | Means and methods for treatment evaluation |
| 20030157526 | Identification of genetic markers of biological age and metabolism |
| 20030152923 | Classifying cancers |
| 20030135033 | Compounds and methods for the identification and/or validation of a target |
| 20030134280 | Identifying drugs for and diagnosis of benign prostatic hyperplasia using |
| | gene expression profiles |
| 20030119062 | Detection of differential expression of protein using gel-free proteomics |
| 20030106074 | Collections of transgenic animal lines (living library) |
| 20030104463 | Identification of pharmaceutical targets |
| 20030104419 | Method of classifying a thyroid carcinoma using differential gene |
| | expression |
| 20030104394 | Method and system for gene expression profiling analysis utilizing |
| | frequency domain transformation |
| 20030092009 | Profiling tumor specific markers for the diagnosis and treatment of |
| | neoplastic disease |
| 20030086914 | Method and device for inducing biological processes by micro-organs |
| 20030082647 | Transporter protein |
| 20030077740 | Presenilin/Crk binding polypeptides (PCBP) and methods of use thereof |
| 20030068318 | Treatment of uterine serous papillary cancer |
| 20030051266 | Collections of transgenic animal lines (living library) |
| 20030041345 | Receptor-like protein kinases from nicotiana tabacum |
| 20030036079 | Gene expression alterations underlying the retardation of aging by caloric |
| | restriction in mammals |
| 20030032030 | Identification of gene expression alterations underlying the aging process in |
| | mammals |
| 20030013093 | Expression of genes in diabetes mellitus and insulin resistance |
| 20020179097 | Method for providing clinical diagnostic services |
| 20020169562 | Defining biological states and related genes, proteins and patterns |
| 20020168664 | Automated pathway recognition system |
| 20020151051 | Compositions and methods for isolating genes comprising subcellular |
| | localization sequences |
| 20020110820 | Genetic markers for tumors |
| 20020081614 | Functional genomics using zinc finger proteins |
| 20020076730 | Methods for Identifying Potential Therapeutic Agents for Treatment of |
| | Osteoporosis Using Mitogenic Indices |
| 20020039739 | Expression monitoring for gene function identification |
| 20020038471 | Use of VLCFAE for identifying herbicidally active compounds |
| 20020028454 | Expression monitoring for gene function identification |
| 20020012938 | Detection of epithelial dysplasia |
| 20020001797 | Method for detecting endocrine disrupting action of a test substance |
| 20010039539 | Database assisted experimental procedure |
| 20010016318 | Methods for extrating similar expression patterns and related biopolymers |

Documents concerning differential gene expression
Patents

| PAT. NO. | Title |
|---|---|
| 7,031,843 | Computer methods and systems for displaying information |
| | relating to gene expression data |
| 7,003,403 | Quantifying gene relatedness via nonlinear prediction of gene |
| 6,996,477 | Computational subtraction method |
| 6,593,084 | Carcinogen assay |
| 6,511,802 | Solid phase selection of differentially expressed genes |
| 6,503,717 | Methods of using randomized libraries of zinc finger |
| | proteins for the identification of gene function |
| 6,355,423 | Methods and devices for measuring differential |
| | gene expression |
| 6,342,376 | Two-color differential display as a method for detecting |
| | regulated genes |
| 6,057,111 | Gene identification method |
| 5,604,118 | Eukaryotic expression vector system |

Published Patent Applications

| Publ. No. | Title |
|---|---|
| 20060094018 | Discovery and a method for the early detection of pancreatic cancer and |
| | other disease conditions |
| 20060088876 | Method for the early detection of breast cancer, lung cancer, pancreatic |
| | cancer and colon polyps, growths and cancers as well as other |
| | gastrointestinal disease conditions and the preoperative and postoperative |
| | monitoring of transplanted organs from the donor and in the recipient and |
| | their associated conditions related and unrelated to the organ transplantation |
| 20060047697 | Microarray database system |
| 20060036368 | Drug discovery methods |
| 20060029960 | Method for the early detection of pancreatic cancer and other |
| | gastrointestinal disease conditions |
| 20060024693 | Identification of genes or polypeptides the expression of which correlates to |
| | fertility, ovarian function and/or fetal/newborn viability |
| 20050287544 | Gene expression profiling of colon cancer with DNA arrays |
| 20050191634 | Genes for diagnosing colorectal cancer |
| 20050181399 | Methods for enhanced detection & analysis of differentially expressed |
| | genes using gene chip microarrays |
| 20050112630 | Diagnosis, prognosis and identification of potential therapeutic targets of |
| | multiple myeloma based on gene expression profiling |
| 20050112584 | Methods for evaluating tissue pair combinations for use in nucleic acid |
| | array technology |
| 20050089899 | Identification of an ERBB2 gene expression signature in breast cancers |
| 20050084850 | Bone marrow secreted proteins and polynucleotides |
| 20050079509 | Methods for identifying suitable nucleic acid normalization probe |
| | sequences for use in nucleic acid arrays |
| 20040229292 | Use of FGF-18 in the diagnosis and treatment of memory disorders |
| 20040229224 | Allele-specific expression patterns |
| 20040220125 | Biosynthetic platform for cardioprotective gene expression using immature |
| | heart tissue |
| 20040219569 | Gene identification method |
| 20040219532 | Internal references measurements |
| 20040214203 | Genes related to sensitivity and resistance to chemotherapeutic drug |
| | treatment |
| 20040166503 | Methods for gene expression profiling |
| 20040146894 | Methods of diagnosing and treating stress urinary incontinence |
| 20040133355 | Methods and compositions utilizing evolutionary computation techniques |
| | and differential data sets |
| 20040101846 | Methods for identifying suitable nucleic acid probe sequences for use in |
| | nucleic acid arrays |
| 20040067500 | Compositions and methods relating to the peroxisomal proliferator activated |
| | receptor-alpha mediated pathway |
| 20040038324 | Epothilone resistant cell lines |
| 20040009523 | Diagnosis, prognosis and identification of potential therapeutic targets of |
| | multiple myeloma based on gene expression profiling |
| 20030232364 | Diagnosis, prognosis and identification of potential therapeutic targets of |
| | multiple myeloma based on gene expression profiling |
| 20030198970 | Genostics |
| 20030186252 | Array based hybridization assays employing enzymatically generated |
| | labeled target nucleic acids and compositions for practicing the same |
| 20030180761 | Map-based genome mining method for identifying regulatory loci |
| | controlling the level of gene transcripts and products |
| 20030175753 | Diagnosis, prognosis and identification of potential therapeutic targets of |
| | multiple myeloma based on gene expression profiling |
| 20030134318 | Methods of using randomized libraries of zinc finger proteins for the |
| | identification of gene function |
| 20030003463 | Methods and devices for measuring differential gene expression |
| 20020184656 | In vivo assay system for screening and validation of drugs and other |
| | substances |
| 20020182656 | Methods for regulating vascularization using GEF containing NEK-like |
| | kinase (GNK) |
| 20020164709 | Nucleic acid endocing growth factor protein |
| 20020146691 | Methods of using randomized libraries of zinc finger proteins for the |
| | identification of gene function |
| 20020123083 | Nucleic acid endocing growth factor protein |
| 20020106670 | Methods for identifying novel therapeutic agents |
| 20020102013 | Method for comparing signal arrays in digital images |
| 20020090636 | Two-color differential display as a method for detecting regulated genes |
| 20020090603 | Methods of differentiating and protecting cells by modulating the |
| | P38/MEF2 pathway |
| 20020086288 | Carcinogen assay |
| 20020034800 | Bone marrow secreted proteins and polynucleotides |
| 20020015955 | Computational subtraction method |
| 20010052140 | Methods of selection and development of plants having improved root |
| | quality and root lodging resistance |

Documents concerning regulated genes
Patents

| PAT. NO. | Title |
|---|---|
| 7,008,768 | Method for detecting radiation exposure |
| 6,733,969 | Expression monitoring for gene function identification |
| 6,511,802 | Solid phase selection of differentially expressed genes |
| 6,303,301 | Expression monitoring for gene function identification |
| 6,258,536 | Expression monitoring of downstream genes in the BRCA1 |
| | pathway |
| 6,177,248 | Downstream genes of tumor suppressor WT1 |

Published Patent Applications

| Publ. No. | Title |
|---|---|
| 20060099619 | Detection and quantification of miRNA on microarrays |
| 20060040884 | Antisense oligonucleotides directed to genes regulated by trapoxin-induced |
| | HDAC inhibition |
| 20050260648 | Method for the determination of cellular transcriptional |
| 20050251880 | Methods and compositions for regulating plant stress tolerance |
| 20050208493 | Ordering genes by analysis of expression kinetics |
| 20050079508 | Constraints-based analysis of gene expression data |
| 20040248116 | Prostate cancer expression profiles |
| 20040234979 | Differentiall-expressed and up-regulated polynucleotides and polypeptides |
| | in breast cancer |
| 20040191804 | Method of analysis of a table of data relating to gene expression and |
| | relative identification system of co-expressed and co-regulated groups of |
| | genes |
| 20040185489 | Gene transcription assay method |
| 20040158407 | “Recurrent signature” identifies transcriptional modules |
| 20040146922 | Cellular arrays for the identification of altered gene expression |
| 20040146872 | Fat regulated genes, uses thereof and compounds for modulating same |
| 20040143854 | Method for generating a genetically modified organism |
| 20040142373 | Cellular arrays for the identification of altered gene expression |
| 20040110187 | In vitro transcription assay for T box antitermination system |
| 20040058376 | Expression monitoring for gene function identification |
| 20040033517 | Compositions and methods relating to endothelial cell signaling using the |
| | protease activated receptor (PAR1) |
| 20040013691 | Immunotoxin as a therapeutic agent and uses thereof |
| 20040009485 | Cellular arrays for the identification of altered gene expression |
| 20040005574 | SIR2 activity |
| 20030228579 | Ordering genes by analysis of expression kinetics |
| 20030219736 | Cellular arrays for the identification of altered gene expression |
| 20030218634 | System and methods for visualizing diverse biological relationships |
| 20030215835 | Differentially-regulated prostate cancer genes |
| 20030170713 | Method of detecting androgen-regulated gene |
| 20030170689 | DNA microarrays comprising active chromatin elements and |
| | comprehensive profiling therewith |
| 20030148334 | Differentially-expressed genes and polypeptides in angiogenesis |
| 20020197641 | Method for identifying genes that are upstream regulators of other genes of |
| | interest |
| 20020147327 | Modified steroid hormones for gene therapy and methods for their use |
| 20020074342 | Expression miniarrays and uses thereof |
| 20020042076 | Methods for gene array analysis of nuclear runoff transcripts |
| 20020039739 | Expression monitoring for gene function identification |
| 20020028454 | Expression monitoring for gene function identification |
| 20010051339 | Expression monitoring of downstream genes in the BRCA1 pathway |
| 20010039013 | P53-regulated genes |

Additional Documents concerning use of gene expression results and/or information
Patents

| PAT. NO. | Title |
|---|---|
| 7,056,674 | Prediction of likelihood of cancer recurrence |
| 6,996,476 | Methods and systems for gene expression analysis |
| 6,964,850 | Identification and monitoring and treatment of disease and characterization of |
| | biological condition using gene expression profiles |
| 6,960,439 | Identification and monitoring and treatment of disease and characterization of |
| | biological condition using gene expression profiles |
| 6,727,066 | Genes expressed in treated human C3A liver cells |
| 6,544,742 | Detection of genes regulated by EGF in breast cancer |
| 6,468,476 | Methods for using co-regulated genesets to enhance detection and |
| | classification of gene expression patterns |
| 6,316,197 | Method of diagnosing of exposure to toxic agents by measuring distinct |
| | pattern in the levels of expression of specific genes |
| 6,228,589 | Measurement of expression profiles in toxicity determination |

Published Patent Applications

| Publ. No. | Title |
|---|---|
| 20060088836 | Methods and compositions for diagnosing and monitoring transplant |
| | rejection |
| 20060084096 | Biomarkers and expression profiles for toxicology |
| 20060078941 | Gene expression profiling in primary ovarian serous papillary tumors and |
| | normal ovarian epithelium |
| 20060078921 | Biomarkers and expression profiles for toxicology |
| 20060047697 | Microarray database system |
| 20060024692 | Method for diagnosing non-small cell lung cancers |
| 20060008804 | Marker Genes |
| 20050260659 | Compositions and methods for breast cancer prognosis |
| 20050244872 | Breast cancer gene expression markers |
| 20050170372 | Methods and systems for profiling biological systems |
| 20050164272 | Genes differentially expressed in secretory versus proliferative |
| | endometrium |
| 20050048542 | Expression profile algorithm and test for cancer prognosis |
| 20050015206 | Nucleic acid detection assay control genes |
| 20040076974 | Liver necrosis predictive genes |
| 20040038225 | Methods and compositions for categorizing patients |
| 20040018525 | Methods and compositions for the prediction, prognosis, prevention, and |
| | treatment of malignant neoplasma |

### Example 2

### Breast Cancer

Certain embodiments of the present invention concern the use of improved gene expression results in accordance with the present invention in the methods described in U.S. Pat. No. 7,056,674.

As described in the '674 patent, certain embodiments concern a method of predicting the likelihood of long-term survival of a cancer patient without the recurrence of cancer. The method involves knowing (e.g., by determining) the expression level of one or more prognostic RNA transcripts or their expression products in a cancer cell obtained from the patient, normalized against a control transcript or transcripts (e.g., the expression level of all RNA transcripts or their products in the cancer cell, or of a reference set of RNA transcripts or their expression products). The prognostic RNA transcript can be a transcript of one or more genes selected from the group consisting of B_Catenin; BAG 1; BIN 1; BUB 1; C20_orfl; CCNB1; CCNE2; CDC20; CDH1; CEGP1; CIAP1; cMYC; CTSL2; DKFZp586M07; DR5; EpCAM; EstR1; FOXM1; GRB7; GSTM1; GSTM3; HER2; HNRPAB; ID1; IGF1R; ITGA7; Ki_{—}67; KNSL2; LMNB1; MCM2; MELK; MMP12; MMP9; MYBL2; NEK2; NME1; NPD009; PCNA; PR; PREP; PTTG1; RPLPO; Src; STK15; STMY3; SURV; TFRC; TOP2A; and TS. Expression of one or more of BUB1; C20_orf1; CCNB1; CCNE2; CDC20; CDH1; CTSL2; EpCAM; FOXM1; GRB7; HER2; HNRPAB; Ki.sub.—67; KNSL2; LMNB1; MCM2; MELK; MMP12; MMP9; MYBL2; NEK2; NME1; PCNA; PREP; PTTG1; Src; STK15; STMY3; SURV; TFRC; TOP2A; and TS indicates a decreased likelihood of long-term survival without cancer recurrence. Expression of one or more of BAG1; BCatenin; BIN1; CEGP1; CIAP1; cMYC; DKFZp586M07; DR5; EstR1; GSTM1; GSTM3; ID1; IGF1R; ITGA7; NPD009; PR; and RPLPO indicates an increased likelihood of long-term survival without cancer recurrence.

More specifically as described in the '674 patent, the embodiments concern a method of preparing a personalized genomics profile for a patient diagnosed with an ER positive breast cancer, by (a) subjecting RNA extracted from breast cancer cells obtained from the patient to gene expression analysis; (b) determining the expression level in the tissue of the RNA transcripts of GRB7 and STMY3, where the expression level is normalized against a control gene or genes and optionally is compared to the amount found in a breast cancer reference tissue set; and (c) creating a report summarizing the data obtained by the gene expression analysis, where the report includes a prediction of the likelihood of long term survival of the patient in which expression of GRB7 and STMY3 indicates a decreased likelihood of long-term survival without breast cancer recurrence.

Likewise, the embodiments obtained by apply the present invention to the methods of the '674 patent also include a method of predicting the likelihood of long-term survival of an ER positive breast cancer patient (e.g., a patient diagnosed with an invasive ER positive breast cancer) without the recurrence of breast cancer, where the method includes determining the expression level of the RNA transcripts of GRB7 and STMY3 or their expression products in an ER positive breast cancer cell obtained from the patient, normalized against the expression level of a control gene or genes (e.g., all RNA transcripts or their products in the ER positive breast cancer cell, or of a reference set of RNA transcripts or their expression products); where expression of GRB7 and STMY3 indicates a decreased likelihood of long-term survival without breast cancer recurrence. The method can include subjecting the expression level data to statistical analysis, and determining whether the likelihood of such long-term survival has increased or decreased.

Use of the present methods for providing improved results and application in these methods provides better (e.g., more reliable) gene expression results and therefore improved correlation between disease and particular transcripts and/or better prognosis.

As described in the '674 patent, the embodiments are exemplified in a Phase II Study of Gene Expression in 242 Malignant Breast Tumors. A gene expression study was designed and conducted with the primary goal to molecularly characterize gene expression in paraffin-embedded, fixed tissue samples of invasive breast ductal carcinoma, and to explore the correlation between such molecular profiles and disease-free survival.

Study Design: Molecular assays were performed on paraffin-embedded, formalin-fixed primary breast tumor tissues obtained from 252 individual patients diagnosed with invasive breast cancer. All patients were lymph node-negative, ER-positive, and treated with Tamoxifen. Mean age was 52 years, and mean clinical tumor size was 2 cm. Median follow-up was 10.9 years. As of Jan. 1, 2003, 41 patients had local or distant disease recurrence or breast cancer death. Patients were included in the study only if histopathologic assessment, performed as described in the Materials and Methods section, indicated adequate amounts of tumor tissue and homogeneous pathology.

Materials and Methods: Each representative tumor block was characterized by standard histopathology for diagnosis, semi-quantitative assessment of amount of tumor, and tumor grade. When tumor area was less than 70% of the section, the tumor area was grossly dissected and tissue was taken from 6 (10 micron) sections. Otherwise, a total of 3 sections (also 10 microns in thickness each) were prepared. Sections were placed in two Costar Brand Microcentrifuge Tubes (Polypropylene, 1.7 mL tubes, clear). If more than one tumor block was obtained as part of the surgical procedure, the block most representative of the pathology was used for analysis.

Gene Expression Analysis: mRNA was extracted and purified from fixed, paraffin-embedded tissue samples, and prepared for gene expression analysis. Molecular assays of quantitative gene expression were performed by RT-PCR, using the ABI PRISM 7900™. Sequence Detection System™. (Perkin-Elmer-Applied Biosystems, Foster City, Calif., USA). ABI PRISM 7900.™. consists of a thermocycler, laser, charge-coupled device (CCD), camera and computer. The system amplifies samples in a 384-well format on a thermocycler. During amplification, laser-induced fluorescent signal is collected in real-time through fiber optics cables for all 384 wells, and detected at the CCD. The system includes software for running the instrument and for analyzing the data.

Analysis and Results: Tumor tissue was analyzed for 187 cancer-related genes and 5 reference genes. Adequate RT-PCR profiles were obtained from 242 of the 252 patients. The threshold cycle (CT) values for each patient were normalized based on the median of the 7 reference genes for that particular patient. Clinical outcome data were available for all patients from a review of registry data and selected patient charts. Outcomes were classified as:

Event: Alive with local, regional or distant breast cancer recurrence or death due to breast cancer.

No Event: Alive without local, regional or distant breast cancer recurrence or alive with contralateral breast cancer recurrence or alive with non-breast second primary cancer or died prior to breast cancer recurrence.

Analysis was performed by: A. determination of the relationship between normalized gene expression and the binary outcomes of 0 or 1; B. Analysis of the relationship between normalized gene expression and the time to outcome (0 or 1 as defined above) where patients who were alive without breast cancer recurrence or who died due to a cause other than breast cancer were censored. This approach was used to evaluate the prognostic impact of individual genes and also sets of multiple genes.

Analysis of Patients with Invasive Breast Carcinoma by Binary Approach: In the first (binary) approach, analysis was performed on all 242 patients with invasive breast carcinoma. A t test was performed on the groups of patients classified as either no recurrence and no breast cancer related death at 10 years, versus recurrence, or breast cancer-related death at 10 years, and the p-values for the differences between the groups for each gene were calculated.

Based on the data obtained, the expression of any of the following genes in breast cancer indicates a reduced likelihood of survival without cancer recurrence: C20_orf1; CCNB1; CDC20; CDH1; CTSL2; EpCAM; GRB7; HER2; KNSL2; LMNB1; MCM2; MMP9; MYBL2; NEK2; PCNA; PREP; PTTG1; STMY3; SURV; TS; MELK, while the expression of any of the following genes in breast cancer indicates a better prognosis for survival without cancer recurrence: BAG1; BCatenin; CEGP1; CIAP1; cMYC; DKFZp586M07; EstR1; GSTM1; GSTM3; ID1; ITGA7; PR.

Analysis of Multiple Genes and Indicators of Outcome: Two approaches were taken in order to determine whether using multiple genes would provide better discrimination between outcomes. First, a discrimination analysis was performed using a forward stepwise approach. Models were generated that classified outcome with greater discrimination than was obtained with any single gene alone. According to a second approach (time-to-event approach), for each gene a Cox Proportional Hazards model (see, e.g. Cox, D. R., and Oakes, D. (1984), Analysis of Survival Data, Chapman and Hall, London, N.Y.) was defined with time to recurrence or death as the dependent variable, and the expression level of the gene as the independent variable. The genes that have a p-value<0.05 in the Cox model were identified. For each gene, the Cox model provides the relative risk (RR) of recurrence or death for a unit change in the expression of the gene. One can choose to partition the patients into subgroups at any threshold value of the measured expression (on the CT scale), where all patients with expression values above the threshold have higher risk, and all patients with expression values below the threshold have lower risk, or vice versa, depending on whether the gene is an indicator of bad (RR>1.01) or good (RR<1.01) prognosis. Thus, any threshold value will define subgroups of patients with respectively increased or decreased risk.

Based on the data obtained, the expression of any of the following genes in breast cancer indicates a reduced likelihood of survival without cancer recurrence: GRB7; SURV; LMNB1; MYBL2; HER2; MELK; C20_orf1; PTTG1; BUB1; CDC20; CCNB1; STMY3; KNSL2; CTSL2; MCM2; NEK2; Ki.sub.-67; CCNE2; TOP2A-4; PCNA; PREP; FOXM1; NME1; STK15; HNRPAB; MMP9; TS; Src; MMP12; TFRC, and the expression of any of the following genes in breast cancer indicates a better prognosis for survival without cancer recurrence: PR; GSTM1; DR5; CEGP1; BAG1; EstR1; DKFZp586M07; BIN1; NPD009; RPLPO; GSTM3; IGF1R.

The binary and time-to-event analyses, with few exceptions, identified the same genes as prognostic markers. For example, comparison of Tables 1 and 2 shows that 10 genes were represented in the top 15 genes in both lists. Furthermore, when both analyses identified the same gene at [p<0.10], which happened for 26 genes, they were always concordant with respect to the direction (positive or negative sign) of the correlation with survival/recurrence. Overall, these results strengthen the conclusion that the identified markers have significant prognostic value.

Multivariate Gene Analysis of 242 Patients with Invasive Breast Carcinoma; For Cox models comprising more than two genes (multivariate models), stepwise entry of each individual gene into the model is performed, where the first gene entered is pre-selected from among those genes having significant univariate p-values, and the gene selected for entry into the model at each subsequent step is the gene that best improves the fit of the model to the data. This analysis can be performed with any total number of genes. In the analysis the results of which are shown below, stepwise entry was performed for up to 10 genes.

Multivariate analysis was performed using the following equation:
RR=exp[coef(geneA).times.Ct(geneA)+coef(geneB).times.Ct(geneB)+coef(geneC).times.Ct(geneC)+ . . . ].

In this equation, coefficients for genes that are predictors of beneficial outcome are positive numbers and coefficients for genes that are predictors of unfavorable outcome are negative numbers. The “Ct” values in the equation are .DELTA.Cts, i.e. reflect the difference between the average normalized Ct value for a population and the normalized Ct measured for the patient in question. The convention used in the present analysis has been that .DELTA.Cts below and above the population average have positive signs and negative signs, respectively (reflecting greater or lesser mRNA abundance). The relative risk (RR) calculated by solving this equation will indicate if the patient has an enhanced or reduced chance of long-term survival without cancer recurrence.

A multivariate stepwise analysis, using the Cox Proportional Hazards Model, was performed on the gene expression data obtained for all 242 patients with invasive breast carcinoma. The following ten-gene set has been identified by this analysis as having particularly strong predictive value of patient survival: GRB7; LMNB1; ER; STMY3; KLK10; PR; KRT5; FGFR1; MCM6; SNRPF. In this gene set ER, PR, KRT5 and MCM6 contribute to good prognosis, while GRB7, LMNB1, STMY3, KLK10, FGFR1, and SNRPF contribute to poor prognosis.

The methods described in the '674 patent are illustrative of determination and/or use of the correlation between the molecular gene expression profiles of any infectious or non-infectious disease or pathologic state and disease-free survival for patients who have been treated or untreated. As recognized, corresponding gene markers for the disease or condition of interest are used for diagnosis, selection of treatment, evaluation of treatment course and/or outcome prognosis, and/or prognosis of disease or condition recurrence or non-recurrence, and/or of patent condition and/or morbidity and/or mortality. Other disease or conditions include, but are not limited to other forms of cancer, such as other forms of breast cancer, lung cancers, leukemias, lymphomas, prostate cancers, cervical cancers, thyroid cancers, and skin cancers.

### Example 3

### Changes in Gene Expression in Granulocytic Cells

Embodiments of the present invention are also illustrated by application of the present improved gene expression results and profiles to the methods described in U.S. Pat. No. 6,365,352, which is incorporated herein by reference in its entirety. The methods described therein include a method to identify granulocytic cell genes (e.g., cytokine genes, genes encoding cell surface receptors and genes encoding intermediary signaling molecules) that are differentially expressed upon exposure to a pathogen by preparing a gene expression profile of a granulocytic cell population exposed to a pathogen and comparing that profile to a profile prepared from quiescent granulocytic cells, thereby identifying cDNA species, and therefore genes, which are expressed de novo upon neutrophil contact with a pathogen.

Similarly provided is a method to identify granulocytic cell genes that are differentially expressed in response to a sterile inflammatory disease by preparing a gene expression profile of a granulocytic cell population isolated from a subject exhibiting the symptoms of a sterile inflammatory disease and comparing that profile to a profile prepared from granulocytic cells isolated from a normal granulocytic cell population. cDNA species, and therefore genes, which are differentially expressed in the granulocytic cells of a subject exhibiting the symptoms of a sterile inflammatory disease are thereby identified.

The present invention also includes a method to identify granulocytic cell genes that are differentially expressed upon exposure of a granulocytic cell population to an agonist (pro-inflammatory molecule) by preparing a gene expression profile of a granulocytic cell population contacted with an agonist and comparing that profile to a profile prepared from noncontacted granulocytic cells, thereby identifying cDNA species, and therefore genes, which are expressed de novo in the granulocytic cells contacted with the agonist are thereby identified.

The present invention further includes a method to identify a therapeutic or prophylactic agent that modulates the response of a granulocyte population to a pathogen, comprising the steps of preparing a first gene expression profile of a quiescent granulocyte population, preparing a second gene expression profile of a granulocyte population exposed to a pathogen, treating said exposed granulocyte population with the agent, preparing a third gene expression profile of the treated granulocyte population, comparing the first, second and third gene expression profiles and identifying agents that modulate the response of a granulocyte population to the pathogen.

Another aspect of the invention is a method to identify a therapeutic agent that modulates the expression of genes in a granulocyte population found in a subject having Another aspect of the invention includes a method to identify a therapeutic or prophylactic agent that modulates the response of a granulocyte cell population in a subject having a sterile inflammatory disease, comprising the steps of preparing a first gene expression profile of a granulocyte population in a subject having a sterile inflammatory disease, treating the granulocyte population with the agent, preparing a second gene expression profile of the treated granulocyte population, comparing the first and second gene expression profiles with the gene expression profile of a normal granulocyte population and identifying agents that modulate the expression of genes whose transcription levels are altered in the granulocyte population of the subject as compared with normal granulocyte population.

A further aspect of the present invention is a method to identify a therapeutic or prophylactic agent that modulates the response of a granulocytic population to an agonist (pro-inflammatory molecule), comprising the steps of preparing a first gene expression profile of a quiescent granulocyte population, preparing a second gene expression profile of a granulocyte population exposed to an agonist, treating the exposed granulocyte population with the agent, preparing a third gene expression profile of the treated granulocyte population, comparing the first, second and third gene expression profiles and identifying agents that modulate the response of a granulocytic population exposed to an agonist.

The present invention also includes a method of diagnosing the exposure of a subject to a pathogen, comprising the steps of preparing a first gene expression profile of a granulocyte population from the subject, comparing the first gene expression profile to a second gene expression profile of a granulocyte population exposed to that pathogen and to a third gene expression profile of a normal granulocyte preparation and diagnosing whether the subject has been exposed to a pathogen.

Another aspect of the invention includes a method of diagnosing a sterile inflammatory disease in a subject, comprising the steps of preparing a first gene expression profile of a granulocyte population from the subject, comparing the first gene expression profile to at least one second gene expression profile from a granulocyte population from a subject having a sterile inflammatory disease and to a third gene expression profile of a normal granulocyte preparation and thereby determining if the subject has a sterile inflammatory disease.

The present invention also includes a method of identifying new bacterial virulence factor genes by preparing a first gene expression profile of a quiescent granulocyte population, preparing a second gene expression profile of a granulocyte population exposed to a virulent or avirulent bacterial strain, preparing a third gene expression profile from a granulocyte population exposed to a bacterial strain with a mutation in a putative bacterial virulence factor gene, comparing the first, second and third gene expression profiles and identifying a bacterial virulence factor gene.

Another aspect of the invention is a composition comprising a grouping of nucleic acids that correspond to at least a part of one or more of the genes whose expression levels are modulated in a granulocyte population that has been exposed to a pathogen, these nucleic acids being affixed to a solid support.

Lastly, an aspect of the invention is a composition comprising a grouping of nucleic acids that correspond to at least part of one or more genes whose expression levels are modulated in a granulocyte population found in a subject having a sterile inflammatory disease, these nucleic acids being affixed to a solid support.

### '352 Patent Example 1

Production of gene expression profiles generated from cDNAs made with RNA isolated from neutrophils exposed to virulent and avirulent bacteria: Expression profiles of RNA expression levels from neutrophils exposed to various bacteria offer a powerful means of identifying genes that are specifically regulated in response to bacterial infection. As an example, the production of expression profiles from neutrophils exposed to virulent and avirulent E. coli and Y. pestis allow the identification of neutrophil genes that are specifically regulated in response to bacterial infection.

Neutrophils were isolated from normal donor peripheral blood following the LPS-free method. Peripheral blood was isolated using a butterfly needle and a syringe containing 5 cc ACD, 5 cc of 6% Dextran (in normal saline). After 30 minutes of settling, plasma was collected and HBSS Hank's balcinceal salt solution (without Ca.sup.++ or Mg.sup.++) was added to a total volume of 40 ml. The plasma was centrifuged (1500 rpm, for 15 m at 4.degree. C.), the supernatant decanted and cold HBSS added to resuspend the cells. The cell suspension was then layered onto a cold Ficoll Hypaq, centrifuged at 500.times.g for 30 m at 4.degree. C. The pellet contains polymorphonuclear neutrophils. Neutrophils can also be isolated by other commonly used methods such as those disclosed in Current Protocols of Immunology (John Wiley & Sons, Inc.), Babior et al. (1981) In: Leokocyte Function, Cline, M. J. Ed., p.1-38 (Church Livingstone, N.Y.), and Haslett et al. (1985) Am. J. Pathol. 119:101-110.

Following isolation, neutrophils were incubated with E. coli or Y. pestis. Before incubation, bacteria are harvested and washed in phosphate buffered saline and opsonized either autologous human serum or complement factor C7 deficient human serum (SIGMA). Incubation was at a ratio of approximately a PMN: bacteria ratio of 1:20 in RPMI 1640 (HEPES buffered) with heat inactivated Fetal Bovine Serum at 37.degree. C. with gentle mixing in a rotary shaker bath.

As controls, neutrophils were incubated with either bacterial lipopolysaccharide (LPS) or latex beads. LPS was added to approximately 3.38×10.sup.8 cells in 100 ml of RPMI Roswell Park. Memorial Institute containing 6% autologous serum to a final concentration of 1 ng/ml to 1.mu.g/l. Incubation proceeded for 30 or 120 minutes with gentle rotation in disposable polycarbonate Erlenmeyer flasks at 37.degree. C. After incubation, the cells were spun down and washed once with HBSS.

Total cellular RNA was prepared from untreated and treated neutrophils are described above using the procedure of Newburger et al. (1981) J. Biol. Chem. 266(24):16171-7 and Newburger et al. (1988) Proc. Natl. Acad Sci USA 85:5215-5219. Ten micrograms of total RNA, the amount obtainable from about 3.times.10.sup.6 neutrophils, is sufficient for a complete set of cDNA expression profiles.

Synthesis of cDNA was performed as previously described by Prashar et al. in WO 97/05286 and in Prashar et al. (1996) Proc. Natl. Acad. Sci. USA 93:659-663. Briefly, cDNA was synthesized according to the protocol described in the GIBCO/BRL kit for cDNA synthesis. The reaction mixture for first-strand synthesis included 6 μg of total RNA, and 200 ng of a mixture of 1-base anchored oligo(dT) primers with all three possible anchored bases with sequences as specified in the '352 patent along with other components for first-strand synthesis reaction except reverse transcriptase. This mixture was incubated at 65 degree C. for 5 m, chilled on ice and the process repeated. Alternatively, the reaction mixture may include 10 g of total RNA, and 2 pmol of 1 of the 2-base anchored oligo(dT) primers such as RP5.0, RP6.0, or RP9.2 (the sequences of which are provided in the '352 patent) along with other components for first-strand synthesis reaction except reverse transcriptase. This mixture was then layered with mineral oil and incubated at 65 degree C. for 7 min followed by 50 degree C. for another 7 min. At this stage, 2 μl of Superscript reverse transcriptase (200 units/μl; GIBCO/BRL) was added quickly and mixed, and the reaction continued for 1 hr at 45-50 degree C. Second-strand synthesis was performed at 16 degree C. for 2 hr. At the end of the reaction, the cDNAs were precipitated with ethanol and the yield of cDNA was calculated. In our experiments, .apprxeq.200 ng of cDNA was obtained from 10 .mu.g of total RNA.

The adapter oligonucleotide sequences were A1 and A2 (the sequences of which are provided in the '352 patent). One microgram of oligonucleotide A2 was first phosphorylated at the 5′ end using T4 polynucleotide kinase (PNK). After phosphorylation, PNK was heated denatured, and 1.mu.g of the oligonucleotide A1 was added along with 10.times. Annealing buffer (1 M NaCl/100 mM Tris-HCI, pH8.0/10 mM EDTA, pH8.0) in a final vol of 20. mu.l. This mixture was then heated at 65 degree C. for 10 min followed by slow cooling to room temperature for 30 min, resulting in formation of the Y adapter at a final concentration of 100 ng/.mu.l. About 20 ng of the cDNA was digested with 4 units of Bgl II in a final vol of 10 .mu.l for 30 min at 37.degree. C. Two microliters (.apprxeq.4 ng of digested cDNA) of this reaction mixture was then used for ligation to 100 ng (.apprxeq.50-fold) of the Y-shaped adapter in a final vol of 5 μl for 16 hr at 15 degree C. After ligation, the reaction mixture was diluted with water to a final vol of 80 μl (adapter ligated cDNA concentration, .apprxeq.50 pg/82l) and heated at 65 degree C. for 10 min to denature T4 DNA ligase, and 2 μl aliquots (with 100 pg of cDNA) were used for PCR.

Set of primers as specified in the '352 patent were used for PCR amplification of the adapter ligated 3′-end cDNAs. To detect the PCR products on the display gel, 24 pmol of oligonucleotide A1 or A1.1 was 5′-end-labeled using 15 μl of [gamma-³²P]ATP (Amersham; 3000 Ci/mmol) and PNK in a final volume of 20 μl for 30 min at 37 degree C. After heat denaturing PNK at 65 degree C. for 20 min, the labeled oligonucleotide was diluted to a final concentration of 2 μM in 80 μl with unlabeled oligonucleotide A1.1 The PCR mixture (20 μl) consisted of 2 μl (.apprxeq.100 pg) of the template, 2 μl of 10 times PCR buffer (100 mM Tris.HCl, pH 8.3/500 mM KCl), 2 μl of 15 mM MgCl.sub.2 to yield 1.5 mM final Mg.sup.2+ concentration optimum in the reaction mixture, 200 μM dNTPs, 200 nM each 5′ and 3′ PCR primers, and 1 unit of Amplitaq Gold. Primers and dNTPs were added after preheating the reaction mixture containing the rest of the components at 85 degree C. This “hot start” PCR was done to avoid artifactual amplification arising out of arbitrary annealing of PCR primers at lower temperature during transition from room temperature to 94 degree C. in the first PCR cycle. PCR consisted of 5 cycles of 94 degree C. for 30 sec, 55 degree C. for 2 min, and 72 degree C. for 60 sec followed by 25 cycles of 94 degree C. for 30 sec, 60 degree C. for 2 min, and 72 degree C. for 60 sec. A higher number of cycles resulted in smeary gel patterns. PCR products (2.5 μl) were analyzed on 6% polyacrylamide sequencing gel. For double or multiple digestions following adapter ligation, 13.2 μl of the ligated cDNA sample was digested with a secondary restriction enzyme(s) in a final vol of 20 μl. From this solution, 3 μl was used as template for PCR. This template vol of 3 μl carried .apprxeq.100 pg of the cDNA and 10 mM MgCl₂ (from the 10.times.enzyme buffer), which diluted to the optimum of 1.5 mM in the final PCR vol of 20 .mu.l. Since Mg²⁺ comes from the restriction enzyme buffer, it was not included in the reaction mixture when amplifying secondarily cut cDNA. Bands were extracted from the display gels as described by Liang et al. (1995 Curr. Opin. Immunol. 7:274-280), reamplified using the 5′ and 3′ primers, and subcloned into pCR-Script with high efficiency using the PCR-Script cloning kit from Stratagene. Plasmids were sequenced by cycle sequencing on an ABI automated sequencer.

FIG. 1 in the '352 patent presents an autoradiogram of the expression profile generated from cDNAs made from RNA isolated from control (untreated) neutrophils (lanes 1, 5, 10, 13, 14 and 16), neutrophils incubated with avirulent E. coli K12 (lanes 2 and 11), virulent Y. pestis D27 (lane 3), avirulent Y. pestis D28 (lane 4), Y. pestis yopB (lane 6), Y. pestis yopE (lane 7), Y. pestis yoph (lane 8), latex beads (lanes 9 and 19), virulent Entero Hemorrhagic E. coli (EHEC) (lane 12), LPS (lane 15), 1 ng/ml LPS for 30 minutes (lane 17), and LPS for 120 minutes (lane 18). The anchoring oligo d(T)18 n1, n2 has A and C at the n1 and n2 positions, respectively. The cDNAs were digested with BglII.

The description provided in this example from the '352 patent is illustrative and can be generalized to encompass any cell type infected or potentially infected with any infectious agent, e.g., virus, prion, bacteria. In such related methods, the respective infected and control cells are used to generate the expression profiles.

### '352 Patent Example 2

Production of gene expression profiles generated from cDNAs made with RNA isolated from neutrophils exposed to virulent and avirulent bacteria and neutrophils exposed to cytokines: Neutrophils were isolated from normal donor peripheral blood following the LPS-free method as set forth in Example 1. Neutrophils were incubated with virulent and avirulent E. coli or Y. pestis, LPS at 1 ng/ml, GM-CSF at 100 units/ml, TNFa at 1000 units/ml, or .gamma.IFN at 100 units/ml. The bacterial cells, LPS or cytokines were added to approximately 3.38.times.10.sup.8 cells in 100 ml of RPMI containing 6% H1 autologous serum. Incubation proceeded for 2 to 4 hours, preferably 2 hours, with gentle rotation in disposable polycarbonate Erienmeyer flasks at 37.degree. C. After incubation, the cells were spun down and washed once with HBSS.

After incubation of the neutrophils, RNA was extracted and the cDNA profiles prepared as described in Example 1. FIG. 2 in the '352 patent is an autoradiogram of the expression profiles generated from cDNAs made with RNA isolated from control (untreated) neutrophils (lanes 1, 5, 10 and 14), neutrophils incubated with avirulent E. coli K12 (lanes 2 and 11), virulent Y. pestis (lanes 3 and 12), avirulent Y. pestis (lanes 4 and 13), 1 ng/ml LPS (lanes 6 and 15), 100 units/ml GM-CSF(lanes 7 and 16), 1000 units/ml TNF.alpha. (lanes 8 and 17) and 100 units/ml.gamma.IFN (lanes 9 and 18). The anchoring oligo d(T)18n1, n2 has A and C at the n1 and n2 positions for lanes 1-9 and G and G at the n1 and n2 for lanes 10-18. The cDNAs were digested with BglII. As shown by FIG. 2 in the '352 patent, the differential expression of mRNA species (as exhibited by cDNA fragments) in neutrophils exposed to virulent and avirulent E. coli and Y. pestis is not equivalent to the differential expression of mRNA species in neutrophils exposed to the various cytokines.

The process described in this Example 2 of the '352 patent can be generalized to apply to any type of cell infected or potentially infected with any type of infectious agent, and further generalizes to encompass the treatment of any cell type with any compound being screened for bioactivity, screening hit, lead compound, bioactive compound, chemical, or drug. In such related methods, the respective infected cells and control cells are used for generating the expression profiles.

### '352 Patent Example 3

Production of gene expression profiles generated from cDNAs made with RNA isolated from neutrophils exposed to bacteria using all 12 possible anchoring oligo d(T) n1, n2: Neutrophils were isolated from normal donor peripheral blood following the LPS-free method. Neutrophils were incubated with E. coli or Y. pestis. After incubation of the neutrophils, RNA was extracted and the cDNA profiles prepared as described in Example 1. FIG. 3 in the '352 patent shows an autoradiogram of the expression profiles generated from cDNAs made with RNA isolated from control (untreated) neutrophils (lane 1), neutrophils incubated with avirulent E. coli K12 (lane 2), virulent Y. pestis (lane 3), avirulent Y. pestis (lane 4). The anchoring oligo d(T)18 n1 and n2 positions are indicated at the top of the figure. The cDNAs were digested with BglII. The '352 patent FIG. 4 shows a summary of genes which are differentially expressed in neutrophils upon exposure to virulent and avirulent E. coli and Y. pestis. Expression patterns are determined by visual examination of the autoradiography gels comparing band intensity between neutrophils exposed to the various bacteria. The autoradiography gels can also be scanned using commonly available equipment, such s a UMAX D-1L scanner. Bands which exhibit altered intensities in gene expression profiles from neutrophils exposed to the various bacteria when compared to the gene expression profile prepared from normal nonexposed neutrophils are then extracted from the display gel as previously described by in Example 1. The isolated fragments are then reamplified using 5′ and 3′ primers, subcloned into pCR-Script (Stratagene) and sequenced using an ABI automated sequencer.

Tables 1 and 2 in the '352 patent provide a summary of cDNA bands which are differentially expressed in response to exposure to E. coli.

### '352 Patent Example 4

Production of expression profiles generated from cDNAs made with RNA isolated from neutrophils isolated from a subject with a sterile inflammatory disease: Neutrophils are isolated from normal donor peripheral blood following the LPS-free method or from subjects exhibiting the symptoms of a sterile inflammatory disease. RNA is extracted and the gene expression profiles prepared as described in Example 1.

To determine the identity of genes (cDNAs) which are differentially expressed in the neutrophils isolated from a subject exhibiting the symptoms of a sterile inflammatory disease, the cDNA profiles prepared from neutrophils from said subject are compared to profiles prepared from neutrophils isolated from the normal donor. Bands which exhibit altered intensities when compared between the gene expression profiles prepared from neutrophils from said subject and profiles prepared from neutrophils isolated from the normal donor are then extracted from the display gel as previously described in Example 1. The isolated fragments are then reamplified using 5′ and 3′ primers, subcloned into pCR-Script (Stratagene) and sequenced using an ABI automated sequencer. Once sequences are obtained which correspond to the bands of interest, the sequences can be compared to known nucleic acid sequences in the available data bases.

This description is illustrative of identifying differentially expressed genes corresponding to a disease or condition, and thus can be generalized to encompass any sterile or non-sterile disease or pathologic state of any kind for any kind of organism or cell. In such corresponding generalized methods, the respective diseased cells and control cells are used for generating the expression profiles.

### '352 Patent Example 5

Method to identify a therapeutic or prophylactic agent that modulates the response of a granulocyte population to a pathogen: The methods set forth in Example 1 offer a powerful approach for identifying therapeutic or prophylactic agents that modulate the expression of neutrophils or other granulocytic cells to a pathogen. For instance, profiles of normal granulocytes and neutrophils or other granulocytes exposed to pathogens such as E. coli, Y. pestis or other pathogenic bacteria are prepared as set forth in Example 1. A profile is also prepared from a granulocyte population that has been exposed to the pathogen in the presence of the agent to be tested. By examining for differences in the intensity of individual bands between the three profiles, agents which up or down regulate genes of interest in the pathogen exposed granulocytes can be identified.

As a specific example, screening for agents which up or down regulate the expression of human pre-B cell enhancing factor (PBEF) can be identified by examining the differences in band intensity between profiles produced from normal granulocytes, granulocytes exposed to the pathogen and granulocytes exposed to the pathogen in the presence of the agent to be tested. As shown in the '352 patent FIG. 4, PBEF is expressed at high levels when exposed to avirulent bacteria, including E. coli K12 and avirulent Y. pestis but is not expressed at high levels in granulocytes exposed to pathogenic Y. pestis. Agents that up regulate PBEF expression as demonstrated by increased band density in the profile produced from granulocytic cells exposed to virulent Y. pestis in the presence of the agent may be useful in modulating the response of neutrophils to bacterial infection.

This description of this example is illustrative of the process of identifying any therapeutic or prophylactic agent for treating any cell or organism infected with any infectious agent or affected by other disease or condition, but using the corresponding cells and therapeutic marker genes.

### '352 Patent Example 6

Method to identify a therapeutic or prophylactic agent that modulates the expression of genes in a granulocyte cell population found in a subject having a sterile inflammatory disease: The methods set forth in Example 4 offer a powerful approach for identifying therapeutic or prophylactic agents that modulate the expression of neutrophils or other granulocytic cells in subjects exhibiting the symptoms of a sterile (non-infectious) inflammatory disease. For instance, gene expression profiles of normal granulocytes and granulocytes from a subject exhibiting the symptoms of a sterile inflammatory disease are prepared as set forth in Examples 1 and 4. A profile is also prepared from a granulocyte population from a subject exhibiting the symptoms of a sterile inflammatory disease that have been exposed to the agent to be tested. By examining these profiles for differences in the intensity of band between the three profiles, agents which up or down regulate genes of interest in a granulocytic population from a subject exhibiting the symptoms of a sterile inflammatory disease can be identified. Agents that up-regulate a gene or genes that are expressed at abnormally low levels in a granulocytic cell population from a subject exhibiting the symptoms of a sterile inflammatory disease compared to a normal granulocytic cell population as well as agents that down regulate a gene or genes that are expressed at abnormally high levels in a granulocytic cell population from a subject exhibiting the symptoms of a sterile inflammatory disease are contemplated.

### '352 Patent Example 7

Production of solid support compositions comprising groupings of nucleic acids that correspond to the genes whose expression levels are modulated in a granulocytic population that has been exposed to a pathogen or nucleic acids that correspond to the genes whose expression levels are modulated in a granulocytic cell population from a subject having a sterile inflammatory disease. As set forth in Examples 1-4, expression profiles from granulocytic cells exposed to a pathogen or granulocytic cells from a subject having a sterile inflammatory disease yield the identity of genes whose expression levels are modulated compared to normal, quiescent granulocytic cells.

Solid supports can be prepared that comprise immobilized representative groupings of nucleic acids corresponding to the genes or fragments of said genes from granulocytic cells whose expression levels are modulated in response to exposure to a pathogen or in a subject having a sterile inflammatory disease. For instance, representative nucleic acids can be immobilized to any solid support to which nucleic acids can be immobilized, such as positively charged nitrocellulose or nylon membranes (see Sambrook et al. (1989) Molecular Cloning: a laboratory manual 2nd., Cold Spring Harbor Laboratory) as well as porous glass wafers such as those disclosed by Beattie (WO 95/11755). Nucleic acids are immobilized to the solid support by well established techniques, including charge interactions as well as attachment of derivatized nucleic acids to silicon dioxide surfaces such as glass which bears a terminal epoxide moiety. A solid support comprising a representative grouping of nucleic acids can then be used in standard hybridization assays to detect the presence or quantity of one or more specific nucleic acid species in a sample (such as a total cellular mRNA sample or cDNA prepared from said mRNA) which hybridize to the nucleic acids attached to the solid support. Any hybridization methods, reactions, conditions and/or detection means can be used, such as those disclosed by Sambrook et al. (1989) Molecular Cloning: a laboratory manual 2nd., Cold Spring Harbor Laboratory, Ausbel et al.(1987) Current Protocols in Molecular Biology, Greene Publishing and Wiley-Interscience or Beattie (WO 95/11755).

One of ordinary skill in the art may determine the optimal number of genes that must be represented by nucleic acid fragments immobilized on the solid support to effectively differentiate between samples, e.g. neutrophils exposed to various pathogens or neutrophils isolated from a patient to be tested for a sterile inflammatory disease. Preferably, at least about 5, 10, 20, 50, 100, 150, 200, 300, 500, 1000 or more preferably, substantially all of the detectable mRNA species in a cell sample or population will be present in the gene expression profile or array affixed to a solid support. More preferably, such profiles or arrays will contain a sufficient representative number of mRNA species whose expression levels are modulated under the relevant infection, disease, screening, treatment or other experimental conditions. In most instances, a sufficient representative number of such mRNA species will be about 1, 2, 5, 10, 15, 20, 25, 30, 40, 50, 50-75 or 100 in number and will be represented by the nucleic acid molecules or fragments of nucleic acid molecules immobilized on the solid support. For example, nucleic acids encoding all or a fragment of one or more of the known genes or previously reported ESTs that are identified in FIG.4 and Tables 1 and 2 of the '352 patent may be so immobilized. The skilled artisan will be able to optimize the number and particular nucleic acids for a given purpose, i.e., screening for modulating agents, identifying activated granulocytes, etc.

The general process of this example generalizes to encompass a solid support containing any group of genes which are characteristic of a particular diseased, or pathologic, or normal, or differentiated, or treated cell type of any kind.

### '352 Patent Example 9

Method of diagnosing exposure of a subject to a pathogen: Expression profiles of RNA expression levels from neutrophils exposed to various bacteria, such as those disclosed in Examples 1 and 3, offer a powerful means to diagnose exposure of a subject to a pathogen. As set forth in Examples 1 and 3, the display patterns generated from cDNAs made with RNA isolated from neutrophils exposed to pathogenic and nonpathogenic E. coli and Y. pestis exhibit unique patterns of cDNA species corresponding to neutrophil mRNA species (genes) whose expression levels are modulated in response to contact of the neutrophils with the bacteria. The contacting of neutrophils with different species of pathogens may result in the production of expression profiles that are unique to each pathogen species or strain. These unique expression profiles are useful in diagnosing whether a subject has been exposed to or is infected with a given pathogen.

Briefly, expression profiles are produced as set forth in Example 1 using neutrophil samples exposed to various pathogens, such as pathogenic strains of E. coli, Y. pestis, Staphylococci, Streptococci or any other bacterial species. Neutrophils are then isolated from the subject to be tested for exposure to a pathogen and an expression profile prepared from the subject's neutrophils by the methods set forth in Example 1. The expression profile prepared from the subject neutrophils can then be compared to the expression profiles prepared from neutrophils exposed to the various pathogen species or strains to determine which expression profile most closely matches the expression profile prepared from the subject, thereby, diagnosing exposure of the subject to a pathogen.

The description of this example can be generalized to encompass the diagnosis of any diseased or pathologic cell state caused by any infectious agent of any kind, as well as to exposure to chemical agents. In such related methods, an expression pattern which is identified as characteristic of exposure to the infectious agent or chemical agent is used for matching with cells which have a putative exposure to determine the exposure or diagnose the infection or exposure.

### '352 Patent Example 10

Method of diagnosing a sterile inflammatory disease in a subject: Expression profiles of RNA expression levels from neutrophils isolated from a subject having a sterile inflammatory disease, such as those disclosed in Example 4, offer a powerful means to diagnose inflammatory diseases such as psoriasis, rheumatoid arthritis, glomerulonephritis, asthma, cardiac and renal reperfusion injury, thrombosis, adult respiratory distress syndrome, inflammatory bowel diseases such as Crohn's disease and ulcerative colitis and periodontal disease. As set forth in Example 4, the gene expression profiles generated from cDNAs made with RNA isolated from neutrophils from subjects having various sterile inflammatory diseases may exhibit unique patterns of cDNA species corresponding to neutrophil mRNA species (genes) whose expression levels are modulated during the inflammatory process. These unique expression profiles are useful in diagnosing whether a subject has a sterile inflammatory disease.

Briefly, expression profiles are produced as set forth in Examples 1 and 4 using neutrophil samples isolated from patients with various sterile inflammatory diseases. Neutrophils are then isolated from the subject to be tested and an expression profile prepared from the subject's neutrophils by the methods set forth in Example 1. The expression profile prepared from the subject neutrophils can then be compared to the expression profiles prepared from neutrophils isolated from patients with various sterile inflammatory diseases to determine which expression profile most closely matches the expression profile prepared from the subject, thereby, diagnosing whether the subject as a sterile inflammatory disease.

As in the preceding examples from the '352 patent, the description of this example can be generalized to encompass the use of gene expression profiles for the diagnosis of any diseased or pathologic cell state for cells of any kind, by using relevant cells for the respective diseased or pathologic cell state and comparing expression profiles of corresponding test cells.

### Example 4

### Applications of Determination of Differential Gene Expression

Additional description of methods, materials, and apparatus which can be used in the present invention and/or in which the present use of improved gene expression results can be applied are described in U.S. Pat. No. 6,884,578. Thus aspects and embodiments of the inventions described therein represent embodiments of the present invention when performed using the present improved gene expression results and/or profiles. The following description is presented in that patent substantially as presented below.

It is well understood by those of skill in the art that variation in the global pattern of gene expression underlies much of the phenotypic diversity among cells. Phenotypic diversity includes both normal variation associated with a change in physiological state and abnormal variation associated with a pharmacological or disease state. One aspect of the current invention differentiates between abnormal variation associated with disease and/or pharmacological state and normal variation associated with physiological state.

A preferred embodiment of the current invention matches an experimental sample to one or more reference samples that match the experimental sample in at least one parameter that is a determinant of physiological status, pharmacological and/or disease status and compares the expression profiles of the experimental and reference samples. In a particularly preferred embodiment, the current invention is used to identify genes that are differentially expressed between matched samples. The embodiments of the present invention are also applicable to diagnosing the disease state of a sample. The embodiments of the present invention are also applicable to characterizing and monitoring disease states. This includes identifying and monitoring the level of a disease state as well as monitoring the effect of therapies on a disease state. The embodiments of the present invention are also applicable to identifying and monitoring drug responses that are specific to a given physiological state. Thus, the present invention is also useful for designing drug therapies that are tailored to the physiological state of a subject. The present invention in one aspect can also be used to identify the physiological or pharmacological state of a sample.

Genes have been identified whose expression is varied greatly (preferably more than 4, 10, 15, or 20 fold) between different physiological states. Differences in physiological states between an experimental and reference sample make it difficult to distinguish between genes that are differentially expressed because of the change in physiological state and genes that are differentially expressed because of another difference between the samples. For example, when comparing an experimental sample to a reference sample to identify genes that are differentially expressed in a disease state, it is preferable to match the physiological state of the experimental and reference samples so that any changes in gene expression that are observed can be attributed to the disease state. Similarly, a difference in disease or pharmacological state can obscure differences in physiological state. In many embodiments the current invention matches the physiological, pharmacological and/or disease states of reference and experimental samples before comparing expression profiles.

Determining the Physiological, Pharmacological and/or Disease State of a Sample:

In one aspect the invention involves gathering information about the physiological, pharmacological and/or disease state of a sample. If, for example, the goal is to diagnose disease in an experimental sample from a human patient one aspect of the invention is to discover information about the physiological and pharmacological state of the sample. Another aspect of the invention is to match the experimental sample to reference samples of similar physiological and pharmacological state. This requires knowledge of the physiological and pharmacological state of the reference sample. In this example, another aspect of the invention that the reference samples are also of known disease status to allow diagnosis of the disease state of the experimental sample. Information about physiological state can be gathered in a variety of ways. If the subject is human, the sex can be obtained for example through an interview, a visual inspection or through karyotyping. Information about genotypic state can be derived by sequence analysis. There are a variety of methods, such as array based analysis, standard sequencing techniques, and other commercially available methods. Information about disease state can be also be obtained through a variety of mechanisms such as identification of symptoms or morphological examination of effected tissue. Determinants of disease state include phenotypic symptoms, level of disease, progress of therapy. It is possible to have more than one disease contributing to the disease state of the sample. Information about pharmacological state can similarly be obtained through a variety of mechanisms. In some circumstances a subject can be interviewed. Under other circumstances it may be necessary to inspect the medical history of the subject or to assay for evidence of drug use through chemical analysis of blood, urine, skin, saliva or hair.

There may be variation in the expression profiles obtained from samples that apparently share a common physiological state. In some embodiments of the invention the best expression profile to use as a reference sample is an average from a plurality of expression profiles of common physiological state. A phenotypic disease state may alter physiological state expression profile (women with a history of sexual abuse have dramatically altered levels of certain hormones—this would be a disease state that might go clinically undetected).

Matching Reference Sample(s) to Experimental Sample(s): According to one aspect of the current invention, samples can be matched by disease state, by physiological state, or by pharmacological state, or any combination of these states. The objective is to minimize differences between the experimental and reference samples. In a particularly preferred embodiment variation between the experimental and reference sample is limited to a single aspect of a disease, physiological or pharmacological state that is being interrogated. In another embodiment the invention removes variation due to one or more indicators of physiological status, pharmacological status or disease status.

In another aspect the invention removes variation due to one or more indicators of physiological status and one or more indicators of pharmacological status. In another aspect the invention removes variation due to one or more indicators of physiological status and one or more indicators of disease status. In another aspect the invention removes variation due to one or more indicators of disease status and one or more indicators of pharmacological status.

In one aspect of the invention the reference sample(s) is selected to match the experimental sample in at least one parameter that is a determinant of physiological state. In this aspect of the invention it is preferable that the reference sample(s) matches the experimental sample in many parameters that are determinants of physiological state. The reference sample and the experimental sample could be from subjects that are similar in age, gender, reproductive status or ethnic origin, any combination of these aspects or other aspects that are determinants of physiological state.

In one aspect of the invention the reference sample is selected to match the experimental sample in at least one aspect of a disease state. In this aspect of the invention it is preferable that the reference sample(s) matches the experimental sample in many parameters that are determinants of disease state.

In another aspect of the invention the reference sample is selected to match the experimental sample in at least one aspect of a pharmacological state. In this aspect of the invention it is preferable that the reference sample(s) matches the experimental sample in many parameters that are determinants of pharmacological state.

Identifying Differentially Expressed Genes from Matched Samples: In one embodiment of the invention matched experimental and reference samples are compared to identify differences. Comparisons that can be made include, but are not limited to: diseased to normal from matching physiological state, diseased to diseased from different physiological states, normal to normal from different physiological states, diseased to diseased from the same physiological state, and normal to normal from the same physiological state. A sample of unknown physiological state can be compared to a plurality of samples of known physiological state to identify the physiological state of the sample.

In many embodiments of the current invention, expression profiles will be compared. In a particularly preferred embodiment expression profiles are compared to identify genes that are differentially expressed between the samples. This embodiment of the invention is useful, for example, for identifying genes that are differentially expressed in a diseased and normal sample or in different levels of disease. Genes that are differentially expressed can be used as diagnostic or prognostic markers or drug/therapy targets or indicators of physiological or pharmacological status. They can be used individually or in sets of, for example, 2, 5, 10, 20, 30, 100, 150, 200, 250, 500, or 1,000 or more. The identified genes can be used to design probes for microarrays.

Diagnosing Disease States: In a particularly preferred embodiment the current invention can be used to diagnose disease. Reference samples are selected to match the experimental sample in physiological and/or pharmacological state and to represent a plurality of different known disease states. The expression profile from the experimental sample is then compared to a plurality of expression profiles from reference samples to identify one or more reference samples that match the expression profile of the experimental sample. The experimental sample is diagnosed with the disease of the matching reference sample(s).

In one aspect of the invention the disease states represented are selected from a subset of diseases that match one or more symptoms in the experimental sample. For example, if the experimental sample is from a 30-year-old female patient with difficulty becoming pregnant, samples from 30-year-old females diagnosed with specific forms of infertility can be chosen as reference samples.

Monitoring Disease States: Following the diagnosis of a particular disease in a patient or subject it is often useful to obtain information about the level of the disease state. If diagnosis is followed by therapy it is also often useful to obtain information about the level of the disease state during and after therapy.

In one aspect the invention is used to identify or characterize the stage of a tumor. Tumorogenic experimental samples are compared to reference samples that are matched to the experimental sample in one or more indicators of physiological or pharmacological status. Reference samples with well characterized tumors are selected. Comparison can be of morphological features or of other biological readouts including expression profiles. The present method stages tumors by comparison to reference samples of matching physiological and/or pharmacological state, thus eliminating gene expression differences that result from differences in physiological and/or pharmacological state that may not be relevant to the disease state.

In one embodiment the present invention can be used for monitoring the disease state of a subject undergoing one or more therapies. This requires the comparison of a sample before treatment with samples following treatment. There may be changes to the physiological state of the patient that occur over the course of the therapy that are unrelated to the therapy. When comparing a sample before treatment to a sample after treatment it will be preferable to identify changes between the samples that result from a change in physiological state. In one aspect the current invention identifies changes that are the result of physiological change rather than therapeutic intervention.

Identifying and Monitoring Drug Responses that are Specific to Physiological State: The current invention can be used to correlate differences in drug efficacy with differences in physiological state. Some drug therapies are highly effective in one patient but ineffective or deleterious in another patient. Differences in drug efficacy may correlate with differences in genotypic state, disease state or physiological state.

The current invention can be used to identify changes in gene expression following drug treatment that are specific to a physiological state. This could facilitate the discovery/design of therapies that are specific for the physiological state of the patient. Drug therapies will have different effects depending on the physiological status of subject. Some drug therapies have different side effects in different physiological states. Some drug therapies have different efficacies in men and women; in particular many are less effective in women than in men. In a preferred embodiment the current invention is used to identify drug effects that are specific to women. In another preferred embodiment the method is used to identify drug effects that are specific to men.

The invention can also be used to identify therapeutic regimens that are optimized for the physiological state of the patient. Therapeutic treatments ideally impart maximal disease reduction with minimal adverse side effects, but many therapeutic treatments do have undesirable side effects. These side effects may be specific to the physiological state of the sample. The current invention could be used as a tool to design therapeutic regimens that are specific for the physiological state of the subject.

Identifying the Physiological State of an Experimental Sample: A sample for which relatively little information is known about the subject from which the sample was supplied could be compared to a plurality of expression profiles of known physiological status in order to determine the physiological status of the subject. For example a blood or semen sample isolated from a crime scene could be used to obtain information about the physiological status of the criminal, such as age and ethnic origin.

Specific Applications: Those skilled in the art will recognize that in a preferred embodiment, the expression profiles from the reference samples will be input to a database. A relational database is preferred and can be used, but one of skill in the art will recognize that other databases could be used. A relational database is a set of tables containing data fitted into predefined categories. Each table, or relation, contains one or more data categories in columns. Each row contains a unique instance of data for the categories defined by the columns. For example, a typical database for the invention would include a table that describes a sample with columns for age, gender, reproductive status, expression profile and so forth. Another table would describe a disease: symptoms, level, sample identification, expression profile and so forth. See U.S. Ser. No. 09/354,935, which is hereby incorporated by reference in its entirety for all purposes.

In one embodiment the invention matches the experimental sample to a database of reference samples. The database is assembled with a plurality of different samples to be used as reference samples. An individual reference sample in one embodiment will be obtained from a patient during a visit to a medical professional. The sample could be for example a tissue, blood, urine, feces or saliva sample. Information about the physiological, disease and/or pharmacological status of the sample will also be obtained through any method available. This may include, but is not limited to, expression profile analysis, clinical analysis, medical history and/or patient interview. For example, the patient could be interviewed to determine age, sex, ethnic origin, symptoms or past diagnosis of disease, and the identity of any therapies the patient is currently undergoing. A plurality of these reference samples will be taken. A single individual may contribute a single reference sample or more than one sample over time. One skilled in the art will recognize that confidence levels in predictions based on comparison to a database increase as the number of reference samples in the database increases. One skilled in the art will also recognize that some of the indicators of status will be determined by less precise means, for example information obtained from a patient interview is limited by the subjective interpretation of the patient. Additionally, a patient may lie about age or lack sufficient information to provide accurate information about ethnic or other information. Descriptions of the severity of disease symptoms is a particularly subjective and unreliable indicator of disease status.

The database is organized into groups of reference samples. Each reference sample contains information about physiological, pharmacological and/or disease status. In one aspect the database is a relational database with data organized in three data tables, one where the samples are grouped primarily by physiological status, one where the samples are grouped primarily by disease status and one where the samples are grouped primarily by pharmacological status. Within each table the samples can be further grouped according to the two remaining categories. For example the physiological status table could be further categorized according to disease and pharmacological status.

As will be appreciated by one of skill in the art, the present invention may be embodied as a method, data processing system or program products. Examples of computer programs and databases are shown in U.S. Ser. Nos. 09/354,935, 08/828,952, 09/341,302, 09/397,494, 60/220,587, and 60/220,645, which are hereby incorporated by reference in their entireties for all purposes. Accordingly, the present invention may take the form of data analysis systems, methods, analysis software and etc. Software written according to the present invention is to be stored in some form of computer readable medium, such as memory, hard-drive, DVD ROM or CD ROM, or transmitted over a network, and executed by a processor. The present invention also provides a computer system for analyzing physiological states, levels of disease states and/or therapeutic efficacy. The computer system comprises a processor, and memory coupled to said processor which encodes one or more programs. The programs encoded in memory cause the processor to perform the steps of the above methods wherein the expression profiles and information about physiological, pharmacological and disease states are received by the computer system as input. U.S. Pat. No. 5,733,729 illustrates an example of a computer system that may be used to execute the software of an embodiment of the invention. This patent shows a computer system that includes a display, screen, cabinet, keyboard, and mouse. The mouse may have one or more buttons for interacting with a graphic user interface. The cabinet preferably houses a CD-ROM or DVD-ROM drive, system memory and a hard drive which may be utilized to store and retrieve software programs incorporating computer code that implements the invention, data for use with the invention and the like. Although a CD is shown as an exemplary computer readable medium, other computer readable storage media including floppy disk, tape, flash memory, system memory, and hard drive may be utilized. Additionally, a data signal embodied in a carrier wave (e.g., in a network including the internet) may be the computer readable storage medium. The patent also shows a system block diagram of a computer system used to execute the software of an embodiment of the invention. The computer system includes monitor, and keyboard, and mouse. The computer system further includes subsystems such as a central processor, system memory, fixed storage (e.g., hard drive), removable storage (e.g., CD-ROM), display adapter, sound card, speakers, and network interface. Other computer systems suitable for use with the invention may include additional or fewer subsystems. For example, another computer system may include more than one processor or a cache memory. Computer systems suitable for use with the invention may also be embedded in a measurement instrument. The embedded systems may control the operation of, for example, a GeneChip.RTM. Probe array scanner as well as executing computer codes of the invention.

Computer methods can be used to measure the variables and to match samples to eliminate gene expression differences that are a result of differences that are not of interest. For example, a plurality of values can be input into computer code for one or more of a: physiological, pharmacological or disease states. The computer code can thereafter measure the differences or similarities between the values to eliminate changes not attributable to a value of interest. Examples of computer programs and databases that can be used for this purpose are shown U.S. Ser. Nos. 09/354,935, 08/828,952, 09/341,302, 09/397,494, 60/220,587, and 60/220,645, which are hereby incorporated by reference in their entireties.

In one aspect of the invention, microarrays will be used to measure expression profiles. Microarrays are particularly well suited because of the reproducibility between different experiments. DNA microarrays provide one method for the simultaneous measurement of the expression levels of large numbers of genes. Each array consists of a reproducible pattern of thousands of different DNAs attached to a solid support. Labeled RNA or DNA is hybridized to complementary probes on the array and then detected by laser scanning. Hybridization intensities for each probe on the array are determined and converted to a quantitative read-out of relative gene expression levels. The data can be further analyzed to identify expression patterns and variation that correlates with the biological state of the sample. (See U.S. Pat. Nos. 6,040,138, 5,800,992 and 6,020,135, 6,033,860 and U.S. Ser. No. 09/341,302, all of which are incorporated herein by reference.) High-density oligonucleotide arrays are particularly useful for monitoring the gene expression pattern of a sample. In one approach, total mRNA isolated from the sample is converted to labeled cRNA and then hybridized to an array such as a GeneChip.RTM. oligonucleotide array. Each sample is hybridized to a separate array. Relative transcript levels are calculated by reference to appropriate controls present on the array and in the sample. See Mahadevappa, M. & Warrington, J. A. Nat. Biotechnol. 17, 1134-1136 (1999) which is hereby incorporated by reference in its entirety for all purposes.

Characterization of Biological Status in Females: The current invention is particularly useful when applied to analysis of experimental samples from female subjects. Women differ from men in the physiological indicator of gender, which contributes to an as yet uncharacterized level of differential gene expression. In addition, there is a tremendous amount of normal variation between female subjects and between different samples from the same female subject. In particular, the female reproductive system and the menstrual cycle add an additional level of physiological variation to the analysis of samples derived from female subjects. As part of a monthly cycle the lining of the female uterus, the endometrium, undergoes a cycle of controlled tissue remodeling unparalleled in other organs. This cycle is presumably driven by changes in gene expression.

Physiological variation between women and men complicates the design of effective therapies for women and the monitoring of therapeutic treatments in women. It is currently well accepted that gender differences result in extensive disparity in the ways males and females respond to therapeutic treatments for a variety of non-gender specific diseases including heart disease and stroke. The reasons for these differences, however, are not well understood, but the menstrual cycle is likely to be at least partially responsible. Much of the research into novel drugs and therapeutic treatments is done using male test subjects. Therefore, there is a great need in the art for methods of incorporating information about the physiological state of a patient into the diagnosis and management of diseases.

Gender differences in the efficacy of drug therapy have been appreciated for many years, but little has been done to investigate these differences. It is believed that hormonal fluctuations within the menstrual cycle may be a primary cause of gender specific drug response. A systematic investigation of the physiological variation throughout the menstrual cycle, both under normal physiological conditions and in response to drug treatment, would be beneficial.

In one embodiment, the current invention correlates information about variation in gene expression with variation in gender. Male and female samples that are matched in other indicators of physiological state are compared to identify genes that are differentially expressed. For example a healthy 30-year-old male of similar, i.e., European, descent could be compared to a healthy 30-year-old female of European descent to identify genes that are differentially expressed between the two physiological conditions. In a further embodiment the current invention could also be used to monitor changes in pharmacological status resulting from drug treatments, taking normal physiological variation into account. For example, the subjects in the first example could be compared again following therapeutic treatment. The genes that were identified in the first example would be compared or subtracted from the genes identified in the second example to identify genes that are differentially expressed as a result of the therapy.

In another aspect, the current invention diagnoses diseases of the female reproductive system. Many disorders of the female reproductive system have relatively poor methods of diagnosis and prognosis and many are typically diagnosed based simply on patient perception, which tends to be unreliable. For example, pre-menstrual syndrome effects large numbers of women, but is typically diagnosed only when other explanations for the observed symptoms are eliminated. More reliable methods of diagnosis such as the use of gene expression profiles for diagnosis and prognosis have been complicated by the changes in gene expression that accompany the normal physiological variation of the system.

Menopause is a woman's final menstrual period, but currently the actual event can be determined only in retrospect, after she has not had a period for 12 continuous months. Menopause can occur naturally any time between the mid-30s through the late 50s, but can also be brought on prematurely by events such as gynecological surgery, cancer therapy and certain illnesses and diseases. The current invention can be used to determine a molecular profile consistent with a diagnosis of menopause that would allow earlier diagnosis.

In one embodiment the current invention diagnosis diseases of the female reproductive organs. An expression profile from an experimental sample is compared to expression profiles from reference samples that match the experimental sample in physiological state. The reference samples represent a plurality of different disease states that effect the uterus and the experimental sample is identified as being of the disease state of the reference sample that is the closest match. The samples can be derived from, for example, endometrial tissue, myometrial tissue, and/or uterine tissue.

In one aspect, a database of reference samples could be comprised of expression profiles from endometrial samples and data points identifying the physiological, pharmacological and/or disease state of the samples. These reference samples would be from many different individuals representing many different physiological, pharmacological and/or disease states. The reference samples can be derived from for example: normal tissue at different stages of development and differentiation, tissues affected with a variety of pathological conditions, including but not limited to, premenstrual syndrome, PMDD, stress urinary incontinence, polycystic ovarian disease, endometriosis, endometrial cancer, infertility, hormone imbalance, and tissue subjected to a variety of perturbations including but not limited to hormone replacement therapy, or chemical contraception. In one preferred embodiment, reference samples will be taken from individuals during routine doctor visits. In one embodiment the reference samples would represent different physiological states of the menstrual cycle including but not limited to the secretory and proliferative stages of the endometrium.

Providing a Nucleic Acid Sample: One of skill in the art will appreciate that it is desirable to have nucleic samples containing target nucleic acid sequences that reflect the transcripts of interest. Therefore, suitable nucleic acid samples may contain transcripts of interest. Suitable nucleic acid samples, however, may contain nucleic acids derived from the transcripts of interest. As used herein, a nucleic acid derived from a transcript refers to a nucleic acid for whose synthesis the mRNA transcript or a subsequence thereof has ultimately served as a template. Thus, a cDNA reverse transcribed from a transcript, an RNA transcribed from that cDNA, a DNA amplified from the cDNA, an RNA transcribed from the amplified DNA, etc., are all derived from the transcript and detection of such derived products is indicative of the presence and/or abundance of the original transcript in a sample. Thus, suitable samples include, but are not limited to, transcripts of the gene or genes, cDNA reverse transcribed from the transcript, cRNA transcribed from the cDNA, DNA amplified from the genes, RNA transcribed from amplified DNA, and the like.

Transcripts, as used herein, may include, but not limited to pre-mRNA nascent transcript(s), transcript processing intermediates, mature mRNA(s) and degradation products. It is not necessary to monitor all types of transcripts to practice this invention. For example, one may choose to practice the invention to measure the mature mRNA levels only.

In one embodiment, such sample is a homogenate of cells or tissues or other biological samples. Preferably, such sample is a total RNA preparation of a biological sample. More preferably in some embodiments, such a nucleic acid sample is the total mRNA isolated from a biological sample. Those of skill in the art will appreciate that the total MRNA prepared with most methods includes not only the mature MRNA, but also the RNA processing intermediates and nascent pre-mRNA transcripts. For example, total MRNA purified with poly (T) column contains RNA molecules with poly (A) tails. Those poly A+ RNA molecules could be mature mRNA, RNA processing intermediates, nascent transcripts or degradation intermediates.

Biological samples may be of any biological tissue or fluid or cells. Frequently the sample will be a “clinical sample” which is a sample derived from a patient. Clinical samples provide rich sources of information regarding the various states of genetic network or gene expression. Some embodiments of the invention are employed to detect mutations and to identify the function of mutations. Such embodiments have extensive applications in clinical diagnostics and clinical studies. Typical clinical samples include, but are not limited to, sputum, blood, blood cells (e.g., white cells), tissue or fine needle biopsy samples, urine, peritoneal fluid, and pleural fluid, or cells therefrom. Biological samples may also include sections of tissues such as frozen sections taken for histological purposes. Another typical source of biological samples are cell cultures where gene expression states can be manipulated to explore the relationship among genes. In one aspect of the invention, methods are provided to generate biological samples reflecting a wide variety of states of the genetic network.

One of skill in the art would appreciate that it is desirable to inhibit or destroy RNase present in homogenates before homogenates can be used for hybridization. Methods of inhibiting or destroying nucleases are well known in the art. In some preferred embodiments, cells or tissues are homogenized in the presence of chaotropic agents to inhibit nuclease. In some other embodiments, RNases are inhibited or destroyed by heat treatment followed by proteinase treatment.

Methods of isolating total mRNA are also well known to those of skill in the art. For example, methods of isolation and purification of nucleic acids are described in detail in Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993) and Chapter 3 of Laboratory Techniques in Biochemistry and Molecular Biology: Hybridization With Nucleic Acid Probes, Part I. Theory and Nucleic Acid Preparation, P. Tijssen, ed. Elsevier, N.Y. (1993)).

In a preferred embodiment, the total RNA is isolated from a given sample using, for example, an acid guanidinium-phenol-chloroform extraction method and polyA.sup.+ mRNA is isolated by oligo dT column chromatography or by using (dT)n magnetic beads (see, e.g., Sambrook et al., Molecular Cloning: A Laboratory Manual (2nd ed.), Vols. 1-3, Cold Spring Harbor Laboratory, (1989), or Current Protocols in Molecular Biology, F. Ausubel et al., ed. Greene Publishing and Wiley-Interscience, New York (1987)). See also PCT/US99/25200 for complexity management and other sample preparation techniques, which is hereby incorporated by reference in its entirety.

Frequently, it is desirable to amplify the nucleic acid sample prior to hybridization. One of skill in the art will appreciate that whatever amplification method is used, if a quantitative result is desired, care must be taken to use a method that maintains or controls for the relative frequencies of the amplified nucleic acids to achieve quantitative amplification. Methods of “quantitative” amplification are well known to those of skill in the art. For example, quantitative PCR involves simultaneously co-amplifying a known quantity of a control sequence using the same primers. This provides an internal standard that may be used to calibrate the PCR reaction. The high density array may then include probes specific to the internal standard for quantification of the amplified nucleic acid. Other suitable amplification methods include, but are not limited to polymerase chain reaction (PCR) (Innis, et al., PCR Protocols. A guide to Methods and Application. Academic Press, Inc. San Diego, (1990)), ligase chain reaction (LCR) (see Wu and Wallace, Genomics, 4:560 (1989), Landegren, et al., Science, 241:1077 (1988) and Barringer, et al., Gene, 89:117 (1990), transcription amplification (Kwoh, et al., Proc. Natl. Acad. Sci. USA, 86:1173 (1989)), and self-sustained sequence replication (Guatelli, et al., Proc. Nat. Acad. Sci. USA, 87:1874 (1990)).

Cell lysates or tissue homogenates often contain a number of inhibitors of polymerase activity. Therefore, RT-PCR typically incorporates preliminary steps to isolate total RNA or mRNA for subsequent use as an amplification template. One tube mRNA capture methods may be used to prepare poly(A)+RNA samples suitable for immediate RT-PCR in the same tube (Boehringer Mannheim). The captured mRNA can be directly subjected to RT-PCR by adding a reverse transcription mix and, subsequently, a PCR mix. In a particularly preferred embodiment, the sample mRNA is reverse transcribed with a reverse transcriptase and a primer consisting of oligo dT and a sequence encoding the phage T7 promoter to provide single stranded DNA template. The second DNA strand is polymerized using a DNA polymerase. After synthesis of double-stranded cDNA, T7 RNA polymerase is added and RNA is transcribed from the cDNA template. Successive rounds of transcription from each single cDNA template result in amplified RNA. Methods of in vitro polymerization are well known to those of skill in the art (see, e.g., Sambrook, supra).

It will be appreciated by one of skill in the art that the direct transcription method described above provides an antisense (aRNA) pool. Where antisense RNA is used as the target nucleic acid, the oligonucleotide probes provided in the array are chosen to be complementary to subsequences of the antisense nucleic acids. Conversely, where the target nucleic acid pool is a pool of sense nucleic acids, the oligonucleotide probes are selected to be complementary to subsequences of the sense nucleic acids. Finally, where the nucleic acid pool is double stranded, the probes may be of either sense as the target nucleic acids include both sense and antisense strands.

The protocols cited above include methods of generating pools of either sense or antisense nucleic acids. Indeed, one approach can be used to generate either sense or antisense nucleic acids as desired. For example, the cDNA can be directionally cloned into a vector (e.g., Stratagene's p Bluscript II KS (+) phagemid) such that it is flanked by the T3 and T7 promoters. In vitro transcription with the T3 polymerase will produce RNA of one sense (the sense depending on the orientation of the insert), while in vitro transcription with the T7 polymerase will produce RNA having the opposite sense. Other suitable cloning systems include phage lambda vectors designed for Cre-loxP plasmid subcloning (see e.g., Palazzolo et al., Gene, 88:25-36 (1990)).

Other analysis methods that can be used in the present invention include electrochemical denaturation of double stranded nucleic acids, U.S. Pat. Nos. 6,045,996 and 6,033,850, the use of multiple arrays (arrays of arrays), U.S. Pat. No. 5,874,219, the use of scanners to read the arrays, U.S. Pat. Nos. 5,631,734; 5,744,305; 5,981,956 and 6,025,601, methods for mixing fluids, U.S. Pat. No. 6,050,719, integrated device for reactions, U.S. Pat. No. 6,043,080, integrated nucleic acid diagnostic device, U.S. Pat. No. 5,922,591, and nucleic acid affinity columns, U.S. Pat. No. 6,013,440. All of the above patents are hereby incorporated by reference in their entireties.

As described in U.S. Pat. No. 6,884,578, a specific application of the above methods is a method for diagnosing endometrial cancer in an endometrial tissue sample, where the method involves obtaining a gene expression profile from the endometrial tissue sample where expression of the following genes is measured: KIAA0367, KIAA0119, platelet activating factor acetylhydrolase 1B gamma-subunit, UDP-galactose transporter related ioszyme, HMG-1 and Lamin B; and identifying the sample as being cancerous if KIAA0367 expression is downregulated as compared to normal (e.g., a normal control sample) and KIAA0119, platelet activating factor acetylhydrolase 1B gamma-subunit, UDP-galactose transporter related ioszyme, HMG-1 and Lamin B expression is upregulated as compared to normal (e.g., a normal control sample).

### Example 5

### Assessing and Treating Cancer

Another example of methods to which the present improved gene expression results and profiles apply is provided in U.S. Patent Publ. 20050003422, which is incorporated herein by reference in its entirety. The description and methods therein are representative of methods in which expression levels of a marker or set of markers is indicative of patient response to a treatment, e.g., a drug treatment.

As described in Example 1 in that publication, microarray analysis was used for determining expression levels. Such expression level determinations and resulting applications such as treatment evaluation are improved by using improved gene expression results and profiles according to the present invention, and therefore represent embodiments of the present invention.

As described in that publication, bone marrow samples were collected from consenting patients both before and after treatment with Zamestra™ farnesyl transferase inhibitor (FTI) (active ingredient, (R)-6-[amino(4-chlorophenyl)(1-methyl-1H-imidazol-5-yl)methyl]-4-(3-chlor-ophenyl)-1-methyl-2(1H)-quinolinone), diluted with PBS and centrifuged with Ficoll-diatrizoate (1.077 g/ml). White blood cells were washed twice with PBS, resuspended in FBS with 10% DMSO and immediately frozen at −80 degree C. Samples were thawed at 37 degree C. and 10 times volume of RPMI with 20% FBS was added drop-wise over a period of 5 min. Cells were centrifuged at 500 g for 10 min and resuspended in 10 ml PBS with 2 mM EDTA and 0.5% BSA. Samples were then passed through a 70 μM filter (Becton Dickinson Labware, Franklin lakes, N.J.) to remove any cell clumps. Cell viability was determined by trypan blue dye exclusion assay. The mean viability of the bone marrow samples upon thawing was 35% (range 0-96%). Due to the relatively low number of viable cells present the samples were not further enriched for myeloid cells. Approximately 2×10⁵ cells were double labeled with CD33-FITC and CD34-PE antibodies (Becton Dickinson Biosciences Pharmingen, San Diego, Calif.) and FACS analysis was performed.

Total RNA was extracted from cell samples using the RNeasy Kit (Qiagen, Santa Clarita, Calif.). Synthesis of cDNA and cRNA were performed according to Affymetrix (Santa Clara, Calif.) protocols. Two rounds of linear amplification were performed since the RNA yield for several samples was less than 1 μg. For hybridization, 11 μg of cRNA were fragmented randomly by incubation at 94 degree C. for 35 min in 40 mM Tris-acetate, pH 8.1, 100 mM potassium acetate, and 30 mM magnesium acetate. Fragmented cRNA was hybridized to U133A arrays at 45 degree C. for 16 hr in a rotisserie oven set at 60 rpm. Following hybridization, arrays were washed (with 6 times SSPE and 0.5 times SSPE containing Triton X-100 (0.005%)), and stained with streptavidin-phycoerythrin (SAPE; Molecular Probes, Eugene, Oreg.). Quantification of bound labeled probe was conducted using the Agilent G2500A GeneArray scanner (Agilent Technologies, Palo Alto, Calif.).

The total fluorescence intensity for each array was scaled to the uniform value of 600. Chip performance was quantitated by calculating a signal to noise ratio (raw average signal/noise). Chips were removed from further analysis if their signal-to-noise ratio was less than 5. Genes were only included in further analysis if they were called “present” in at least 10% of the chips. Approximately 12,000 Affymetrix probe sets remained following this cut-off. The quality of the gene expression data was further controlled by identifying outliers based on principal components analysis and by analyzing the normal distributions of the gene intensities.

Chi-squared tests and Student's t-test were used to identify correlations between patient response and patient co-variates, mutational status, CD33 and CD34 antigen expression, leukemic blast counts, and gene expression. To identify genes that could predict response with high sensitivity, a percentile analysis was employed. For example, genes that were up- or down-regulated in all responders compared to at least 40% of non-responders were identified. Genes that did not reveal significant p-values (P<0.05) based on a two-tailed Student's t-test (unequal variance) were removed. The predictive value of the top gene(s) was analyzed by a leave-one-out cross validation method. Here, one sample was removed from the data set and the marker was reselected from the 12,000 genes. The predictive value of this gene was then tested on the left-out sample using a linear discriminant analysis. Sensitivity was calculated as the number of true positives divided by the sum of true positives plus false negatives. Specificity was calculated as the number of true negatives divided by the sum of true negatives and false positives. Positive predictive value was calculated as the number of true positives divided by the sum of true positives and false positives. Negative predictive value was calculated as the number of true negatives divided by the sum of false negatives and true negatives.

Univariate cox proportional hazard models were used to assess the association of each parameter (genes or blast counts) with patient survival outcome. The coefficient estimate of each parameter from the cox model measures the strength of such association. When more than one gene was used a multivariate hazard model was employed. The classifier that distinguishes responders from non-responders was defined as:
b1*x1+b2*x2+b3*x3+
where b1, b2, b3 are coefficient estimates from the cox model, and x2, x2, x3 are standardized parameter values (blast counts or log.sub.10 of gene expression values).

Receiver operator curves (ROC) were used to choose appropriate thresholds for each classifier, requiring a sensitivity of at least 90%. The ROC diagnostic calculates the sensitivity and specificity for each parameter. In addition, gene markers were first ranked for their ability to stratify good outcome from poor outcome using a training set of 29 randomly chosen samples. The predictive value of each gene was then tested on the remaining 29 samples. This allowed for the identification of genes with the most robust predictive values.

Example 4 of the publication description exemplified the identification of genes that are differentially expressed between responders and non-responders. Bone marrow samples were obtained for gene expression analysis from 80 patients prior to drug treatment. Of the 80 base-line samples, 14 were removed from the analysis since they came from non-evaluable patients. Samples were enriched for myeloid cells, processed for messenger RNA (the molecules that encode for gene-specific proteins), and hybridized to the Affymetrix U133A gene chip. 58 of the 66 samples passed additional quality control measures following hybridization to the U133A chip. The gene expression data was integrated with the clinical information and retrospective analyses were performed to identify genes that could stratify responders from non-responders with a high level of sensitivity. Several gene markers were identified that are useful in predicting response to Zarnestra™. (Table 3). In the case of the LBC oncogene (oncoLBC) the predictive value of this gene was calculated for the dataset using a leave-one-out cross validation. The oncoLBC gene expression levels were able to capture all of the clinically identified responders while removing over half of the non-responders. Results were shown in Table 3, with identification of differentially expressed genes. Results of the leave-one-out cross validation using oncoLBC as a marker of response analysis was shown in Table 4.

A survival analysis showed that patients who were classified as responders based on oncoLBC expression significantly outperformed patient classification using the clinical data. This was due to the oncoLBC marker identifying a subset of non-responders with an increased overall survival. Based on the Cox hazard model, combining the oncoLBC and a second gene marker, the aryl hydrocarbon receptor (AHR), increased the specificity and positive predictive value to 75% and 56%, respectively. These results indicated that using either the oncoLBC alone, or in combination with the AHR gene presents an effective array for predicting response to Zamestra™ treatment.

A leave-one-out cross validation using the oncoLBC and AHR as markers was also performed. When using the cut-off to identify the highest sensitivity and best specificity the PPV and sensitivity remained the same. Results of leave-one out cross validation of other gene combinations were shown in Table 5. This illustrates that marker combinations can improve the predictive value of this method.

Furthermore, stratification of patients using the LBC and AHR classifier showed a similar difference in median survival time between the two patient populations compared with using the oncoLBC gene or the clinical response definitions. These results indicated that using either the oncoLBC alone, or in combination with the AHR gene could be used as effective biomarkers for predicting response to ZARNESTRA™ in the current dataset.

A Cox hazard model was used to analyze other combinations of markers in stratifying poor survivors from good survivors as shown in Table 6 in the publication. Here, data from 51 patients was used since only this number of patient samples had CD33 and CD34 antigen levels measured. A sensitivity of greater than 90% was used in determining the appropriate cut-offs for the markers. The use of multiple markers can improve the difference in median survival times of the 2 survival groups.

Example 5 of the publication concerned identification of genes that are differentially expressed between responders and non-responders (Repeat Analysis). As described, supervised analysis was performed using the gene expression data to identify additional genes that were differentially expressed between all responders and at least 40% of non-responders. These criteria were chosen to identify genes that could predict response to Tipifamib with the highest level of sensitivity possible. A total of 19 genes were identified that could stratify responders and non-responders (shown in Tables 7 and 8 of the publication) and that gave significant p-values in the t-test (p<0.05). Interestingly, the genes include those involved in signal transduction, apoptosis, cell proliferation, oncogenesis, and potentially, FTI biology (ARHH, LBC and, IL3RA).

Example 6 of the publication concerned identification of a minimal set of 3 gene markers. To identify a candidate set of gene markers that could predict response to tipifarnib with an improved accuracy compared to LBC alone, LOOCV was used to determine the optimal number of genes. Classifiers were built with increasing number of genes based on t-test p-values, and the error rate of these classifiers was calculated using LOOCV while keeping the sensitivity of predicting response at 100%. It was found that a 3-gene classifier (including LBC, AHR, and MINA53) could predict response with the lowest error rate. This was also seen when a leave-five-out cross validation was performed. When more genes were added the error rate increased indicating that additional genes introduced noise to the classifier. For the 3-gene classifier the LOOCV demonstrated a sensitivity of 86% and specificity of 70% with an overall diagnostic accuracy of 74.

Kaplan-Meier analysis again showed a significant difference in survival between the predicted responder group and the non-responder group. Moreover, if the incorrectly classified non-responders were compared to the correctly classified non-responders, the misclassified non-responders showed a better overall survival. This implies that the gene signature could predict a level of response to therapy that cannot be observed by the clinical criteria. Alternatively, this may allow the gene signature to predict response to FTI treatment thus providing prognostic value. Further analysis carried out in untreated patients can be used to determine the relationship between the gene expression signatures and prognosis.

All patents and other references cited in the specification are indicative of the level of skill of those skilled in the art to which the invention pertains, and are incorporated by reference in their entireties, including any tables and figures, to the same extent as if each reference had been incorporated by reference in its entirety individually.

One skilled in the art would readily appreciate that the present invention is well adapted to obtain the ends and advantages mentioned, as well as those inherent therein. The methods, variances, and compositions described herein as presently representative of preferred embodiments are exemplary and are not intended as limitations on the scope of the invention. Changes therein and other uses will occur to those skilled in the art, which are encompassed within the spirit of the invention, are defined by the scope of the claims.

It will be readily apparent to one skilled in the art that varying substitutions and modifications may be made to the invention disclosed herein without departing from the scope and spirit of the invention. For example, variations can be made to the particular application and the analyses utilized. Thus, such additional embodiments are within the scope of the present invention and the following claims.

The invention illustratively described herein suitably may be practiced in the absence of any element or elements, limitation or limitations which is not specifically disclosed herein. Thus, for example, in each instance herein any of the terms “comprising”, “consisting essentially of” and “consisting of” may be replaced with either of the other two terms. The terms and expressions which have been employed are used as terms of description and not of limitation, and there is no intention that in the use of such terms and expressions of excluding any equivalents of the features shown and described or portions thereof, but it is recognized that various modifications are possible within the scope of the invention claimed. Thus, it should be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims.

In addition, where features or aspects of the invention are described in terms of Markush groups or other grouping of alternatives, those skilled in the art will recognize that the invention is also thereby described in terms of any individual member or subgroup of members of the Markush group or other group.

Throughout this disclosure, various aspects of this invention are presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, unless clearly indicated to the contrary description of a range such as from 1 to 6 should be considered to have specifically disclosed subranges such as from 1 to 3, from 1 to 4, from 1 to 5, from 2 to 4, from 2 to 6, from 3 to 6 etc., as well as each of the individual numbers within that range, for example, 1, 2, 3, 4, 5, and 6. This applies regardless of the breadth of the range. Also, unless indicated to the contrary, where various numerical values or value range endpoints are provided for embodiments, additional embodiments are described by taking any 2 different values as the endpoints of a range or by taking two different range endpoints from specified ranges as the endpoints of an additional range. Such ranges are also within the scope of the described invention and should be considered as specifically disclosed.

Thus, additional embodiments are within the scope of the invention and within the following claims.

1. A method for producing an improved gene expression profile (GEP) for one or more cell samples, comprising:
(a) determining one of more particular gene (PG) improved results (R) for said cell sample; and
(b) compiling said PG IR values to produce one or more forms of improved GEP for said cell sample.

2. (canceled)

3. The method of claim 1, wherein said at least one cell sample comprises a plurality of cell samples.

4. (canceled)

5. The method of claim 1, wherein said one or more particular gene (PG) improved results (IR) comprises IRs for a plurality of different PGs.

6-9. (canceled)

10. The method of claim 1, wherein PGs comprising said cell sample GEP comprise at least one cellular RNA.

11. The method of claim 10, wherein said cellular RNA comprises mRNA.

12. The method of claim 10, wherein said cellular RNA comprises siRNA.

13. The method of claim 10, wherein said cellular RNA comprises miRNA.

14. The method of claim 10, wherein said cellular RNA comprises regulatory RNA.

15-19. (canceled)

20. The method of claim 1, wherein said determining one or more particular gene(PG) improved results(IR) for said cell sample is performed using a microarray assay.

21. The method of claim 1, wherein said determining one or more particular gene(PG) improved results(IR) for said cell sample is performed using RT-PCR.

22. The method of claim 1, wherein said determining one or more particular gene(PG) improved results(IR) for said cell sample is performed using at least one of affinity binding media, nucleae protection, and clone counting methods.

23. A method for identifying a particular cell sample type of interest, comprising
comparing gene expression profiles (GEPs) of at least one cell sample type of interest and at least one reference cell sample type of interest, and
identifying said cell sample type of interest based on best match comparison of the respective GEPs.

24. The method of claim 23, wherein said GEPs comprise expression results for a plurality of PGs.

25. (canceled)

26. The method of claim 23, further comprising
utilizing the method of claim 1 to determine a GEP for one or more cell samples of the said cell sample type of interest or for one or more cell samples of a specified reference cell sample type of interest, or both; and
incorporating said results in at least one GEP.

27-50. (canceled)

51. A method for identifying a set of genes which may be used to identify or characterize a particular cell sample type of interest, comprising
determining improved gene expression results for a plurality of particular genes (PGs) in said particular cell sample type of interest and in at least one reference cell sample type; and
identifying and analyzing PGs which are differentially expressed in the cell sample of interest compared to said reference cell sample type.

52. The method of claim 51, further comprising
selecting at least a subset of said differentially expressed genes as said set of genes which may be used to identify or characterize said cell sample type of interest.

53. The method of claim 51, wherein said improved gene expression results are compiled as a GEP for one or more cell samples of the said cell sample type of interest or for one or more cell samples of a specified reference cell sample type of interest, or both; and
said identifying PGs which are differentially expressed comprises comparing gene expression profiles (GEPs) of at least one cell sample type of interest and at least one reference cell sample type of interest.

54-82. (canceled)

83. An improved set of cell sample type discrimination gene set identifier nucleic acid molecules, comprising;
a set of nucleic acid molecules which provide specific detection of individual particular genes identified by the method of claim 51 which reliably, selectively, and specifically identify individual cell samples of the type of interest, or distinguish said cell sample type of interest from at least one specific reference cell sample type, or both, based on improved gene expression results.

84-92. (canceled)

93. A method for identifying improved sets of particular genes for an application utilizing gene expression results, comprising
obtaining improved gene expression results for a least one application pertinent gene: and
selecting a discrimination gene set based on differential gene expression of said gene in at least one application pertinent cell sample type.

94. (canceled)

95. The method of claim 93, wherein said discrimination gene set is selected utilizing the method of claim 51.

96. The method of claim 93, wherein said improved gene expression results are obtained utilizing the method of claim 1.

97-101. (canceled)

102. A method for producing improved results for an application which directly or indirectly utilizes at least one gene expression profile for at least one particular gene, comprising
utilizing at least one improved gene expression profile (GEP) directly or indirectly in said application, thereby producing improved application results.

103. The method of claim 102, wherein said GEP is produced according to claim 1.

104-108. (canceled)

109. An improved method for identifying regulated particular genes (PGs) which are regulated in response to exposure to a particular treatment, comprising
comparing at least one improved gene expression profile (GEP) incorporating improved results for at least one cell sample exposed to said treatment with at least one improved gene expression profile for at least one reference cell sample, thereby identifying PGs with differential expression is said treated cell sample.

110. (canceled)

111. The method of claim 109, wherein a GEP is provided utilizing the method of claim 1.

112-114. (canceled)

115. The method of claim 109, further comprising
exposing at lest one of a plurality of matching cell samples to a treatment of interest thereby forming a treated cell sample, while at least one other of said cell sample portions is not exposed to said treatment of interest, and constitutes said reference sample; and
using the method of claim 1 to produce a GEP for each of said cell samples.

116-119. (canceled)

120. The method of claim 109 wherein at least one treated cell sample comprises a plurality of separate different cell sample types.

121-150. (canceled)

151. A method for producing improved information and results concerning the physiological state of cells in a cell sample of a particular cell type of interest, comprising
utilizing one or more particular physiological state gene expression profiles (PS GEPs) to identify the physiological state of different samples of the particular cell type of interest, wherein particular PS GEPs for the particular cell type of interest selectively distinguish a particular physiological state (PS) for said particular cell type of interest,
wherein said PS GEPs are improved by the incorporation of improved gene expression results and wherein said information and results are improved in one or more of qualitative accuracy, quantitative accuracy, interpretability, producibility, intercomparability, and utility, relative to prior art produced information and results.

152. (canceled)

153. The method of claim 151 wherein the method of claim 1 is utilized to produce one or more physiological state gene expression profiles (PS GEPs) for the particular cell type of interest which selectively distinguish a particular physiological state (PS) for said particular cell type of interest.

154-155. (canceled)

156. A method for producing improved clinical trial information and results which are improved in qualitative accuracy, quantitative accuracy, interpretability, reproducibility, intercomparability, or utility, relative to prior art produced such information and results, for the evaluation of one or more or all of the safety, dose, or efficacy of a drug or bioactive agent(BA), comprising
monitoring one or more improved gene expression profiles (GEPs) for drug or BA treated and untreated particular cell types of interest respectively for the appearance of one or more drug treatment desired effects or undesired effects or both in said treated cell types of interest, wherein said improved GEPs incorporate improved gene expression results.

157-166. (canceled)

167. A method for producing improved information and results concerning the efficacy and toxicity or both or the desired and undesired effects or both, of treatment for a patient being treated with a particular drug or bioactive agent (BA), or with a combination of a plurality of drugs or BAs or both, which is improved in one or more of qualitative accuracy, quantitative accuracy, interpretability, reproducibility, intercomparability, and utility, relative to such prior art produced information and results, comprising
monitoring one or more improved gene expression profiles (GEPs) of patient cell samples for drug or BA treated particular cell types of interest for the appearance of one or more drug treatment desired effects or undesired effects or both in said treated cell types of interest, wherein said improved GEPs incorporate improved gene expression results.

168-177. (canceled)

178. A method for producing improved patient bioactive agent treatment related health care, comprising
utilizing the method of claim 167 to determine the effectiveness of the particular drug or bioactive agent (BA) treatment in a patient, and
selecting a drug or BA treatment utilizing the determination of effectiveness information.

179-187. (canceled)

188. A method for producing improved patient bioactive agent treatment related health care, comprising
selecting treatment for a patient based on comparison of at least one improved GEP for said patient, and at least one reference GEP indicative of patient response to said drug or bioactive agent treatment.

189-201. (canceled)
